(19) United States
(12) Patent Application Publication
(10) Pub. No.: US 2024/0197854 A1
(43) Pub. Date: Jun. 20, 2024

(54) COMPOSITIONS COMPRISING HIV ENVELOPES TO INDUCE HIV-1 ANTIBODIES

(71) Applicant: Duke University, Durham, NC (US)

(72) Inventors: Rory Henderson, Durham, NC (US); Barton F. Haynes, Durham, NC (US)

(21) Appl. No.: 18/274,943

(22) PCT Filed: Jan. 28, 2022
(86) PCT No.: PCT/US22/14321

§ 371 Date: Jul. 28, 2023

Related U.S. Application Data
(60) Provisional application No. 63/142,744, filed on Jan. 28, 2021.

(51) Int. Cl. A61K 39/12; A61K 39/00; A61K 39/39; A61P 31/18; C07K 14/005

(52) U.S. Cl. CPC A61K 39/12; A61K 39/39; A61P 31/18; C07K 14/005; A61K 2039/55555; A61K 2039/575; C12N 2740/16122; C12N 2740/16134

(57) ABSTRACT

This invention provides in general, a composition suitable for use in inducing anti-HIV-1 antibodies, such as immunogenic compositions comprising envelope proteins and nucleic acids to induce cross-reactive neutralizing antibodies and increase their breadth of coverage. The invention also provides methods of inducing such broadly neutralizing anti-HIV-1 antibodies using such compositions.

This application claims the benefit of and priority to U.S. Application Ser. No. 63/142,744, filed Jan. 28, 2021, the contents of which are incorporated by reference herein in its entirety.

This invention was made with government support under 1UM1AI144371 awarded by NIH, NIAID. The government has certain rights in the invention.

### TECHNICAL FIELD

This invention provides in general, a composition suitable for use in inducing anti-HIV-1 antibodies, such as immunogenic compositions comprising envelope proteins and nucleic acids to induce cross-reactive neutralizing antibodies and increase their breadth of coverage. The invention also provides methods of inducing such broadly neutralizing anti-HIV-1 antibodies using such compositions.

### BACKGROUND

The development of a safe and effective HIV-1 vaccine is one of the highest priorities of the scientific community working on the HIV-1 epidemic. While anti-retroviral treatment (ART) has dramatically prolonged the lives of HIV-1 infected patients, ART is not routinely available in developing countries.

### SUMMARY OF THE INVENTION

In some aspects, the invention provides envelope sequence designs comprising amino acid changes at one or more positions in an HIV envelope, wherein these envelope positions are antibody-envelope encounter sites/residues. In some embodiments, the antibody-envelope encounter residue(s) are contact residues. In certain aspects, the invention provides envelope designs comprising amino acid changes (mutations) of antibody contact residues at selected positions, wherein these changes improve binding of these envelopes to antibodies compared to an unmodified parental sequence. The encounter and contact residues are identified based on molecular dynamics simulation based Markov models of antibody variable region association with a portion of the HIV-1 Env termed gp120. Non-limiting embodiments of envelope designs are described in Table 1.

In certain aspects the invention provides a recombinant HIV-1 envelope selected from the envelopes listed in FIG. 7, FIG. 21 or Table 1. In non-limiting embodiments, the envelope is CH848.3.D0949.10.17chim.6R.DS.SOSIP.664_T238K_E241T_N408K_N442A, which is also referred as HV1302206_N442A. In non-limiting embodiments, the envelope is HV1302206. In non-limiting embodiments, the envelope is HV1302209. In non-limiting embodiments the envelope is HV1302212. In non-limiting embodiments, the envelope is HV1302215. In non-limiting embodiments, the envelope is HV1302212_N442A. In some embodiments, any of these envelopes comprise further changes, including without limitation embodiments where certain glycan holes are filled—D230N_H289N_P291S (HXB2 numbering).

In certain aspects the invention provides a composition comprising an envelope selected from the envelopes listed in FIG. 7, FIG. 21 or Table land a carrier, wherein the envelope is a protomer comprised in a trimer. In certain embodiments, the envelope is comprised in a trimer. In certain embodiments, the trimer is multimerized and is comprised in a nanoparticle. In certain embodiments, the nanoparticle is self assembling. In certain embodiments, the nanoparticle is a ferritin nanoparticle.

In certain aspects, the invention provides a composition comprising a nanoparticle and a carrier, wherein the nanoparticle comprises any one of the envelopes from the envelopes listed in FIG. 7, FIG. 21 or Table 1. In certain embodiments, the envelope in the nanoparticle is comprised in a trimer. In certain embodiments, the nanoparticle is a ferritin self-assembling nanoparticle.

In certain embodiments, the nanoparticle comprises multimers of trimers. In certain embodiments, the nanoparticle comprises 1 to 8 trimers.

In certain aspects the invention provides methods of inducing an immune response in a subject comprising administering in an amount sufficient to affect such induction an immunogenic composition comprising any one of the recombinant envelopes the invention or a composition with nanoparticles comprising the envelopes of the invention. In certain aspects the composition is administered as a prime.

In certain embodiments, the composition is administered as a boost.

In certain aspects, the invention provides a nucleic acid encoding any of the recombinant envelopes of the invention. In certain aspects, the invention provides a composition comprising a nucleic acid of the invention and a carrier. In certain embodiments, the nucleic acid is an mRNA, wherein in certain embodiments the mRNA is modified, and/or encapsulated in lipid nanoparticles (LNPs). mRNA modifications include without limitation use of modified nucleosides, e.g. 1-methyl-pseudouridine.

In certain aspects, the invention provides a method of inducing an immune response in a subject comprising administering in an amount sufficient to affect such induction an immunogenic composition comprising the nucleic acid of the invention, including without limitation composition comprising modified mRNAs.

Provided are also immunogenic compositions comprising these envelope sequences and methods for their use.

In certain aspects, the invention provides compositions comprising a selection of HIV-1 envelopes and/or nucleic acids encoding these envelopes as described herein, for example, but not limited to designs as described herein. Without limitations, these selected combinations comprise envelopes which provide representation of the sequence (genetic) and antigenic diversity of the HIV-1 envelope variants which lead to the induction of V3 glycan antibody lineages.

In certain embodiments, these changes/mutations are comprised in a recombinant HIV-1 envelope comprising 17aa V1 region, lacks glycosylation at position N133 and N138 (HXB2 numbering), comprising glycosylation at N301 (HXB2 numbering) and N332 (HXB2 numbering), comprising modifications wherein glycan holes are filled (D230N_H289N_P291S (HXB2 numbering)), comprising the “GDIR” or “GDIK” motifs, or any trimer stabilization modifications, UCA targeting modification, immunogenicity modification, or combinations thereof, for example but not limited to these described in PCT/US2019/049431. In certain embodiments, the recombinant envelope optionally comprises any combinations of these modifications.

In certain embodiments the envelope is any one of the envelopes listed in Table 1, FIG. 7, FIG. 21. In certain embodiments, the envelope is not CH848 10.17 DT variant described previously in WO/2018/161049.

In certain embodiments, the envelope is a protomer which can be comprised in a stable trimer.

In certain embodiments, the envelope comprises additional mutations stabilizing the envelope trimer. In certain embodiments these include, but are not limited to, SOSIP mutations. In certain embodiments mutations are selected from sets F1-F14, VT1-VT8 mutations described herein, or any combination or subcombination within a set. In certain embodiments, the selected mutations are F14. In other embodiments, the selected mutations are VT8. In certain embodiments, the selected mutations are F14 and VT8 combined. F14 and VT8 envelope designs are disclosed in PCT/US2019/049662 which content is incorporated by reference in its entirety.

In certain embodiments, the invention provides a recombinant HIV-1 envelope of FIG. 7, FIG. 21 or Table 1. In certain embodiments, the invention provides a nucleic acid encoding any of the recombinant envelopes. In certain embodiments, the nucleic acid is an mRNA formulated for use as a pharmaceutical composition.

In certain embodiments, the inventive designs comprise specific changes (D230N_H289N_P291S (HXB2 numbering)) which fill glycan holes with the introduction of new glycosylation sites to prevent the binding of strain-specific antibodies that can hinder broad neutralizing antibody development (Wagh, Kshitij et al. “Completeness of HIV-1 Envelope Glycan Shield at Transmission Determines Neutralization Breadth.” Cell reports vol. 25,4 (2018): 893-908.e7. doi: 10.1016/j.celrep.2018.09.087; Crooks, Ema T et al. “Vaccine-Elicited Tier 2 HIV-1 Neutralizing Antibodies Bind to Quaternary Epitopes Involving Glycan-Deficient Patches Proximal to the CD4 Binding Site.” PLOS pathogens vol. 11,5 e1004932. 29 May 2015, doi:10.1371/journal.ppat.1004932).

In certain embodiments, the inventive envelope designs comprise modifications, including without limitation linkers between the envelope and ferritin designed to optimize ferritin nanoparticle assembly.

In certain embodiments, the invention provides envelopes comprising Lys327 (HXB2 numbering) optimized as a prime to initiate a V3 glycan antibody lineage, e.g. DH270 antibody lineage.

In certain embodiments, the invention provides envelopes comprising Lys169 (HXB2 numbering).

In certain embodiments, the invention provides a composition comprising any one of the inventive envelopes or nucleic acid sequences encoding the same. In certain embodiments, the nucleic acid is mRNA. In certain embodiments, the mRNA is comprised in a lipid nano-particle (LNP).

In certain aspects, the invention provides nucleic acids comprising sequences encoding envelopes of the invention. In certain embodiments, the nucleic acids are DNAs. In certain embodiments, the nucleic acids are mRNAs. In certain aspects, the invention provides expression vectors comprising the nucleic acids of the invention.

In certain aspects, the invention provides a pharmaceutical composition comprising mRNAs encoding the inventive antibodies. In certain embodiments, these are optionally formulated in lipid nanoparticles (LNPs). In certain embodiments, the mRNAs are modified. Modifications include without limitations modified ribonucleotides, poly-A tail, 5′ cap.

In certain aspects, the invention provides nucleic acids encoding the inventive envelopes. In non-limiting embodiments, the nucleic acids are mRNA, modified or unmodified, suitable for any use, for example, but not limited to, use as pharmaceutical compositions. In certain embodiments, the nucleic acids are formulated in lipid, such as but not limited to LNPs.

In certain embodiments, the invention provides compositions comprising a nanoparticle which comprises any one of the envelopes of the invention.

In certain embodiments, the invention provides any of the composition described herein, wherein the composition comprises nanoparticles and the nanoparticle is a ferritin self assembling nanoparticle.

In certain embodiments, the invention provides a method of inducing an immune response in a subject comprising administering an immunogenic composition comprising any one of the envelopes of the invention. In certain embodiments, the composition is administered as a prime and/or a boost. In certain embodiments, the composition comprises nanoparticles. In certain embodiments, methods of the invention further comprise administering an adjuvant.

The envelope encounter designs, including without limitation envelopes disclosed in Table 1 and FIG. 7, e.g.HV1301345_T238K_E241T_N353K_N442A, are intended as boosts to CH848.10.17.SOSIP nanoparticles containing mutations to remove potential N-glycosylation sites, N133D and N138T (DT nano-particle construct), with or without glycan holes filled—D230N_H289N_P291S (HXB2 numbering). In non-limiting embodiments, the immunization protocol involves prime/boost immunizations with the DT nanoparticle construct which is to be followed by boosts with the design as a SOSIP trimer or nanoparticle and followed thereafter with the unmutated CH848.10.17.SOSIP—with or without glycan holes filled—D230N_H289N_P291S (HXB2 numbering) as trimer or nanoparticle.

In certain embodiments, the invention provides a composition comprising a plurality of nanoparticles comprising a plurality of the envelopes/trimers of the invention. In non-limiting embodiments, the envelopes/trimers of the invention are multimeric when comprised in a nanoparticle. In some embodiments, the nanoparticle size is suitable for delivery. In non-liming embodiments, the nanoparticles are ferritin based nano-particles.

### BRIEF DESCRIPTION OF THE DRAWINGS

The patent or application file contains at least one drawing executed in color.

FIG. 1A-E. DH270 Lineage. A. DH270 B cell maturation lineage. See “Staged Induction of HIV-1 Glycan-dependent Broadly Neutralizing Antibodies” Mattia Bonsignori et al. Science Translational Medicine Doi: 10.1126/scitranslmed. aai7514, 2017. B. Computationally defined Functional Improbable mutations. See Functional Relevance of Improbable Antibody Mutations For HIV Broadly Neutralizing Antibody Development. Kevin Wiehe, et al. Cell Host and Microbe: 23: 759-765, 2018. C. Mutagenesis to define the minimum improbable and probable mutations. Figure shows that 12 mutations reach 90% of DH270.6 breadth. See Rapid Selection of HIV Envelopes That Bind to Neutralizing Antibody B Cell Lineage Members with Functional Improbable Mutations Olivia Swanson, Doi: https://doi.org/10.1101/2021.01.04.425252, 2021. D. Immunogen Design, Presentation, and Animal Models: Immunogen Testing. E. DH270 Lineage Cryo-EM Structure Determination. The lineage tree begins with the unmutated common ancestor and leads through intermediates (I) to mature DH270 antibodies. Structures determined to date for Env complexes with DH270 lineage antibodies, colors from green to blue (upper branch) and green to orange (lower branch), in addition structures determined for the same Envs to VRC01 (red). Antibodies are bound to HIV-1 virus isolate SOSIP Env from patient CH848 (grey; differing shades indicate distinct Env sequences).

FIGS. 2A-C. HIV-1 Env gp120 Encounter Sites. (A) DH270.6 vs. CH848 gp120 encounter states. The gp120 is depicted as a cartoon representation colored according to sheet (yellow), helix (purple) and loops (light grey and teal). Antibodies are displayed as “clouds” of states with a single antibody shown in cartoon for reference. The HCDR3 is displayed in green. The N156 (orange), N301 (green), N332 (pink), and N442 (cyan) glycans are shown as a stick representation. (B) The CH848 gp120 (orange) with Man9 glycans (grey) highlighting the different encounter regions. Solid outlines indicate experimental and/or simulation observation of an encounter state while dashed lines indicate unobserved but predicted encounter sites. (C) Exchange between states.

FIGS. 3A-J. DH270.6 Encounter State Design Binding Results. A-E) DH270 lineage Antibody vs. CH848 DT SOSIP BLI results for the N442 A mutant and the V4 region mutant (V4 Glyc+Chrg). ND indicates date not available. F-J) DH270 lineage Antibody vs. CH848 non-DT SOSIP BLI results for the N442A mutant and the V4 region mutant (V4 Glyc+Chrg). Data are normalized to the binding response from trimer specific antibody PGT151.

FIG. 4 shows that distant encounter sites enable association.

FIG. 5 shows effect of glycan N442.

FIG. 6 shows an I3 targeting immunogen.

FIG. 7 shows non-limiting embodiments of amino acid sequences of envelopes of the invention. The depicted amino acid sequences do not include a signal peptide sequence. A skilled artisan can readily identify a suitable signal peptide for expression in any expression system. A skilled artisan can readily determine nucleic acids sequences, including optimized and/or modified sequences which encode these amino acid sequences. SEQ ID NOS: [ ] to [ ] in order of appearance.

FIG. 8 shows one embodiment of a design for the production of trimeric HIV-1 Env on ferritin nanoparticles.

FIGS. 9A-E show binding results obtained using biolayer interferometry using human IgG capture tips to immobilize HIV-1 Envelope trimer specific broadly neutralizing antibody (bnAb) PGT151 or DH270 lineage member antibody UCA, I5, I3, I2 and DH270.6. Binding levels for PGT151 were used to normalize binding responses for parent (in this figure HV1301345_N133D_N138T) and design constructs to eliminate active concentration differences between responses for comparison. Figure shows binding of respective antibody UCA, I5, I3, I2 and DH270.6 to a set of envelopes as indicated in the figure. These binding results for the DH270 lineage antibodies beginning from the Unmutated Common Ancestor (UCA, FIG. 9A) and stepping sequentially through the I5 (FIG. 9B), I3 (FIG. 9C), and I2 (FIG. 9D) intermediates and the bnAb DH270.6 (FIG. 9E). Results indicate the HV1301345_N133D_N138T_N442A and HV1301345_N133D_N138T_T238K_E241T_N353K constructs display enhanced binding relative to the HV1301345_N133D_N138T parent construct throughout the lineage (I2 results not determined). Addition of the N442A mutation to the HV1301345_N133D_N138T_T238K_E241T_N353K construct results in enhanced binding at the I3 intermediate.

FIGS. 10A-E show Binding results here were obtained using biolayer interferometry using human IgG capture tips to immobilize HIV-1 Envelope trimer specific broadly neutralizing antibody (bnAb) PGT151 or DH270 lineage member antibody UCA, I5, I3, I2 and DH270.6. Binding levels for PGT151 were used to normalize binding responses for parent (in this figure HV1301345) and design constructs to eliminate active concentration differences between responses for comparison. Figure shows binding of respective antibody UCA, I5, I3, I2 and DH270.6 to a set of envelopes as indicated in the figure. These binding results for the DH270 lineage antibodies beginning from the Unmutated Common Ancestor (UCA, FIG. 10A) and stepping sequentially through I5 (FIG. 10B), I3 (FIG. 10C), and I2 (FIG. 10D) intermediates and the bnAb DH270.6 (FIG. 10E). Results indicate the HV1301345_N442A and HV1301345_T238K_E241T_N353K constructs display enhanced binding relative to the HV1301345 parent construct throughout the lineage. Addition of the N442A mutation to the HV1301345_T238K_E241T_N353K construct results in enhanced binding at the I5 and I3 intermediate with an increased differential at 13.

FIG. 11A-B shows CH848 SOSIP_DH270 Lineage Fab Kinetics (SPR—Biacore S200). FIG. 11B provides are specific run parameters and methodology, also used in FIGS. 12-15.

FIGS. 12A-D show CH848 SOSIP_DH270UCA3 Fab Kinetics. Results for the UCA indicate little change in affinity and kinetics with the design mutations.

FIG. 13A-D show CH848 SOSIP_DH270 13.6 Fab Kinetics. Results for I3 indicate a roughly two-fold improvement in the association rate (ka) over the parent construct, consistent with the BLI binding results. The change is largely the result of the N353K mutations per comparison between HV1301345_N353K and HV1301345_T238K_E241T_N353K.

FIG. 14A-D show CH848 SOSIP_DH270 I2.6 Fab Kinetics. Results for I2 indicate a roughly two-fold improvement in the association rate (ka) over the parent construct, consistent with the BLI binding results. The change is largely the result of the N353K mutations per comparison between HV1301345_N353K and HV1301345_T238K_E241T_N353K.

FIG. 15A-D show CH848 SOSIP_DH270.6 Fab Kinetics. Results for DH270.6 indicate a roughly two-fold improvement in the association rate (ka) over the parent construct, consistent with the BLI binding results. The change is largely the result of the N353K mutations per comparison between HV1301345_N353K and HV1301345_T238K_E241T_N353K.

FIG. 16A-B shows CH848 SOSIP_DH270 Lineage Fab Kinetics (SPR—Biacore S200). FIG. 16B Provides are specific run parameters and methodology, also used in FIGS. 17-20.

FIGS. 17A-D shows CH848 SOSIP_DH270UCA3 Fab Kinetics. Results for the UCA3 indicate a roughly two-fold improvement in the association rate (ka) over the parent construct for the HV1301345_N133D_N138T_N442A, consistent with the BLI binding results. Results for the HV1301345_T238K_E241T_N353K construct are consistent with previous measures.

FIG. 18A-D shows CH848 SOSIP_DH270 13.6 Fab Kinetics. Results for I3 indicate a roughly two-fold improvement in the association rate (ka) over the parent construct for the HV1301345_N133D_N138T_N442A, consistent with the BLI binding results. Results for the HV1301345_T238K_E241T_N353K construct are consistent with previous measures.

FIG. 19A-D shows CH848 SOSIP_DH270 I2.6 Fab Kinetics. Results for I2 indicate a roughly two-fold improvement in the association rate (ka) over the parent construct for the HV1301345_N133D_N138T_N442A, consistent with the BLI binding results. Results for the HV1301345_T238K_E241T_N353K construct are consistent with previous measures.

FIG. 20A-D shows CH848 SOSIP_DH270.6 Fab Kinetics. Results for DH270.6 indicate a roughly two-fold improvement in the association rate (ka) over the parent construct for the HV1301345_N133D_N138T_N442A, consistent with the BLI binding results. Results for the HV1301345_T238K_E241T_N353K construct are consistent with previous measures.

FIG. 21 shows non-limiting embodiments of additional envelope encounter designs. SEQ ID NOS: [ ] to [ ] in order of appearance.

FIG. 22A-B shows Association Rates (kon; linear scale) for various design and DH270 lineage antibodies (x-axis). On rates measured using SPR. SOSIP trimers were captured on the chip using PGT151 with antibody Fabs were passed over the chip. FIG. 22A plot is for immunogens that do not contain the DT mutations. FIG. 22B plot is for immunogens that do contain the N133DN138T (DT) mutations.

FIG. 23A-B shows Dissociation Rates (k_{off}; log scale) for various design and DH270 lineage antibodies (x-axis). Off rates measured using SPR. FIG. 23A plot is for immunogens that do not contain the DT mutations. FIG. 23B Right plot is for immunogens that do contain the DT mutations.

FIG. 24A-B Affinity (K_{D}; log scale) for various design and DH270 lineage antibodies (x-axis). Affinities measured using SPR. FIG. 24A shows plot is for immunogens that do not contain the DT mutations. FIG. 24B plot is for immunogens that do contain the DT mutations.

FIG. 25 shows immunization scheme of IA4 knock-in mice: CH848 10.17 vs. Encounter Design. One group is immunized with CH848 10.17 envelope trimer with glycan holes filled: (D230N_H289N_P291S (HXB2 numbering) “CH848 10.17” or “CH848 10.17 holes filled trimer”. Another group is immunized with HV1302206_N442A.

FIG. 26 shows Next Generation Sequencing (NGS) data from the IA4 knock-in mouse study described in FIG. 25. NGS data for the two critical improbable mutation R98T and L48Y for each mouse in the CH848 10.17 trimer immunized mice (study Mu522 group 1) and the HV1302206_N442A immunized mice (study Mu524 group 3). Data show an increased mean induction of each mutation. An additional study is in progress to confirm these observations.

FIG. 27A-D shows serum binding data from the IA4 Knock-in Mouse study described in FIG. 25. Results from serum binding show an increased mean binding of the CH848.10.17 gp120 monomer (FIG. 27A) and MNgp41 (FIG. 27B) indicating enhanced responses to gp120 and gp41 epitopes. No binding was observed to V3 (FIG. 27C) and V2 (FIG. 27D) peptide.

FIG. 28A-C shows additional serum binding data from the IA4 Knock-in Mouse study described in FIG. 25. Results from serum binding show the HV1302206_N442A induced responses which can interact with CH848.10.17 DT (FIG. 28A), HV1302206_N442A (FIG. 28B), and CH848.0358.80.06 (FIG. 28C) SOSIP trimers.

FIG. 29A-E shows IA4 Knock-in Mouse study Blocking Results. Results from serum blocking show the HV1302206_N442A induced responses which can block N332-glycan interactive bnAb PGT128 but not DH270 for interactions with CH848.10.17. Blocking was observed for several mice at different timepoints for PGT125 and PGT128 for interactions with JR-FL.

FIG. 30 shows IA4 Knock-in Mouse study neutralization. Neutralization data for the CH848 10.17 trimer immunized mice (study Mu522 group 1) and the HV1302206_N442A (study Mu524 group 3). Each row shows data for individual mice in the study as indicated on the right. The identity of the viruses used in the pseudovirus neutralization assays are shown on the bottom. Data show a broadly neutralizing response from mouse 4 in the encounter design immunized set (shown with an arrow).

FIG. 31 shows that multiple antibodies from a HV1302206_N442A immunized mouse Mu.4, contain R98T and L48Y mutation. Data show antibodies isolated from mouse 4 of the HV1302206_N442A (study Mu524 group 3) immunized set. A total of 19 antibodies with the critical improbable R98T were isolated with 10 antibodies also containing the critical improbable L48Y mutation.

FIG. 32 shows scheme for immunization of mice having a knocked-in unmutated common ancestor (UCA) 3 antibody. Mice were immunized first with CH848.3.D0949.10.17_N133D_N138T_D230N_H289N_P291S_E169K_DS.SOSIP_VRCferriti n protein which forms a nanoparticle, and boosted with HV1302206_N442A as a trimer.

FIG. 33A-B show neutralization results in a pseudovirus assay with serum from mice in the study described in FIG. 32. Neutralization results show neutralization of the CH848 10.17 and 10.17 DT for all mice, heterologous neutralization of 92RW020.5 for 4 out of 6 Mice, and heterologous neutralization of 6101.10, 92RW020.5, ZM106F, TRO.11, and T250-4 for one mouse. These results indicate the HV1302206_N442A boost leads to inducing heterologous serum neutralization.

FIG. 34A-D shows NGS data for mice in the following UCA3 knock-in mouse immunization studies—Immunization of UCA3 mice: CH848 10.17 DT Nano Particle with Boost-Immunization 1 was at week 0, Immunization 2 was at week 2, Immunization 3 was at week 4, Immunization 4 was at week 6, Immunization 5 was at week 8, Immunization 6 was at week 10, Immunization 7 was at week 12, Immunization 8 was at week 14:

### Study Mu525 Group 1. Immunizations 1-4:

CH848.3.D0949.10.17_N133D_N138T_D230N_H289N_P291S_E169K_DS.SOSIP_VRCferriti n, all with GLA-SE Adjuvant (5 mcg);

### Study Mu525 Group 2. Immunizations 1-4:

CH848.3.D0949.10.17_N133D_N138T_D230N_H289N_P291S_E169K_DS.SOSIP_VRCferriti n, all with 3M052-Alum Adjuvant (0.5/50 mcg);

### Study Mu563 Group 1. Immunizations 1-4:

CH848.3.D0949.10.17_N133D_N138T_D230N_H289N_P291S_E169K_DS.SOSIP_VRCferriti n Immunizations 5-8: CH0848.3.D0358.80.06CHIM.DS.6R.SOSIP.664/293F, all with 3M052-Alum Adjuvant (0.5/50 mcg);

### Study Mu563 Group 2. Immunizations 1-4:

CH848.3.D0949.10.17_N133D_N138T_D230N_H289N_P291S_E169K_DS.SOSIP_VRCferriti n, Immunizations 5-8: CH0848.3.d0526.25.05 DS.chSOSIP_TPA/293F, all with 3M052-Alum Adjuvant (0.5/50 mcg);

### Study Mu565 Group 1. Immunizations 1-4:

### CH848.3.D0949.10.17_N133D_N138T_D230N_H289N_P291S_E169K_DS.SOSIP_VRCferriti n, Immunizations 5-6:

CH848.3.D0949.10.17_D230N_H289N_P291S_E169K_DS.chSOSIP/293F, Immunizations 7-8:
CH0848.3.D0358.80.06CHIM.DS.6R.SOSIP.664/293F, all with 3M052-Alum Adjuvant (0.5/50 mcg);

### Study Mu565 Group 1. Immunizations 1-4:

### CH848.3.D0949.10.17_N133D_N138T_D230N_H289N_P291S_E169K_DS.SOSIP_VRCferriti n, Immunizations 5-6:

CH848.3.D0949.10.17 D230N_H289N_P291S_E169K_DS.chSOSIP/293F, Immunizations 7-8:
CH0848.3.d0526.25.05 DS.chSOSIP_TPA/293F, all with 3M052-Alum Adjuvant (0.5/50 mcg); Study Mu567 Group 1. Immunizations 1-4:
CH848.3.D0949.10.17_N133D_N138T_D230N_H289N_P291S_E169K_DS.SOSIP_VRCferriti n, Immunizations 5-6: Study Mu567 Group 1: HV1302206_N442A, all with 3M052-Alum Adjuvant (0.5/50 mcg);

### Study Mu577 Group 2. Immunizations 1-4:

CH848.3.D0949.10.17_N133D_N138T_D230N_H289N_P291S_E169K_DS.SOSIP_VRCferriti n, all with 3M052-Alum Adjuvant (0.5/50 mcg)+CH848.3.D0949.10.17_D230N_H289N_P291S_E169K_DS.chSOSIP, all with 3M052-Alum Adjuvant (0.5/50 mcg);

### Study Mu577 Group 3. Immunizations 1-2:

### CH848.3.D0949.10.17_N133D_N138T_D230N_H289N_P291S_E169K_DS.SOSIP_VRCferriti n, Immunizations 3-4:

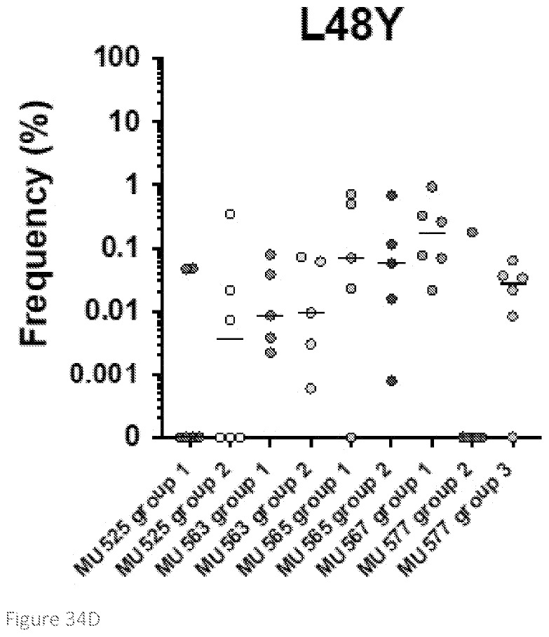

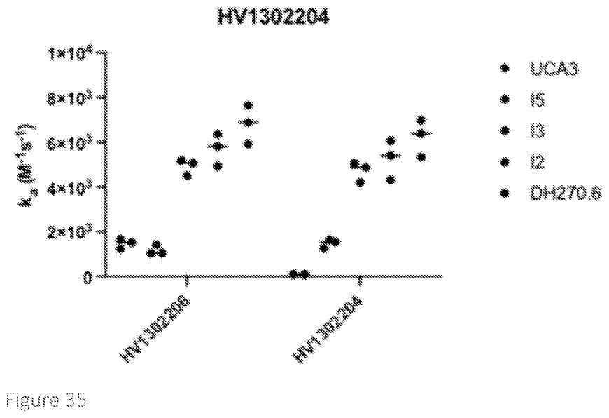

CH848.3.D0949.10.17_N133D_N138T_D230N_H289N_P291S_E169K_DS.SOSIP_VRCferriti n, all with 3M052-Alum Adjuvant (0.5/50 mcg)+CH848.3.D0949.10.17_D230N_H289N_P291S_E169K_DS.chSOSIP, all with 3M052-Alum Adjuvant (0.5/50 mcg). FIG. 34A shows NGS data for G57R improbable mutation. FIG. 34B shows NGS data for R98T improbable mutation. FIG. 34C shows NGS data for the combined G57R, R98T mutations in the heavy chain. FIG. 34D shows NGS data for the L48Y improbable mutation. Next generation sequencing of the heavy and light chains from all comparable studies indicate the HV1302206_N442A (study Mu567 group 1) immunized set are as effective as or better at inducing neutralization critical, improbable mutations G57R, R98T and the combined G57R/R98T mutations in the heavy chain and the L48Y mutation in the light chain.

FIG. 35 shows comparison of kinetics of HV1302206, which has the V4 glycan deletion plus two others, vs. HV1302204, which has only the glycan deletion. This figure shows that these two constructs are similar in their effects. Antibodies on the y-axis are in the following order: UCA, I5, I3, I2 and DH270.6.

### DETAILED DESCRIPTION OF THE INVENTION

The development of a safe, highly efficacious prophylactic HIV-1 vaccine is of paramount importance for the control and prevention of HIV-1 infection. A major goal of HIV-1 vaccine development is the induction of broadly neutralizing antibodies (bnAbs) (Immunol. Rev. 254: 225-244, 2013). BnAbs are protective in rhesus macaques against SHIV challenge, but as yet, are not induced by current vaccines.

The invention provides methods of using optimized envelope immunogens.

In certain aspects, the invention provides compositions for immunizations to induce lineages of broad neutralizing antibodies. In certain embodiments, there is some variance in the immunization regimen; in some embodiments, the selection of HIV-1 envelopes can be grouped in various combinations of primes and boosts, such as nucleic acids, proteins, or combinations thereof. In certain embodiments, the compositions are pharmaceutical compositions which are immunogenic. In certain embodiments, the compositions comprise amounts of envelopes which are therapeutic and/or immunogenic.

In one aspect the invention provides a composition for a prime boost immunization regimen comprising any one of the envelopes described herein, or any combination thereof wherein the envelope is a prime or boost immunogen. In certain embodiments the composition for a prime boost immunization regimen comprises one or more envelopes described herein.

In certain embodiments, the compositions encompass nucleic acid, as DNA and/or RNA, or proteins immunogens, such as alone or in any combination. In certain embodiments, the methods encompass genetic, as DNA and/or RNA, immunization, such as alone or in combination with envelope protein(s).

### Nucleic Acid Sequences

In some embodiments, the immunogens are administered as nucleic acids, including but not limited to mRNAs which can be modified and/or unmodified. See US Pub 20180028645A1, US Pub 20170369532, US Pub 20090286852, US Pub 20130111615, US Pub 20130197068, US Pub 20130261172, US Pub 20150038558, US Pub 20160032316, US Pub 20170043037, US Pub 20170327842, US Pub 20180344838A1 at least at paragraphs [0260]-[0281] for non-limiting embodiments of chemical modifications, wherein each content is incorporated by reference in its entirety.

mRNAs delivered in LNP formulations have advantages over non-LNPs formulations. See US Pub 20180028645A1.

In certain embodiments, the nucleic acid encoding an envelope is operably linked to a promoter inserted an expression vector. In certain aspects, the compositions comprise a suitable carrier. In certain aspects, the compositions comprise a suitable adjuvant.

In certain aspects, the invention provides an expression vector comprising any of the nucleic acid sequences of the invention, wherein the nucleic acid is operably linked to a promoter. In certain aspects, the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects, the invention provides nucleic acids comprising any one of the nucleic acid sequences of invention. In certain aspects, the invention provides nucleic acids consisting essentially of any one of the nucleic acid sequences of invention. In certain aspects, the invention provides nucleic acids consisting of any one of the nucleic acid sequences of invention. In certain embodiments, the nucleic acid of the invention is operably linked to a promoter and is inserted in an expression vector. In certain aspects, the invention provides an immunogenic composition comprising the expression vector.

In certain aspects, the invention provides a composition comprising at least one of the nucleic acid sequences of the invention. In certain aspects, the invention provides a composition comprising any one of the nucleic acid sequences of invention. In certain aspects, the invention provides a composition comprising at least one nucleic acid sequence encoding any one of the polypeptides of the invention.

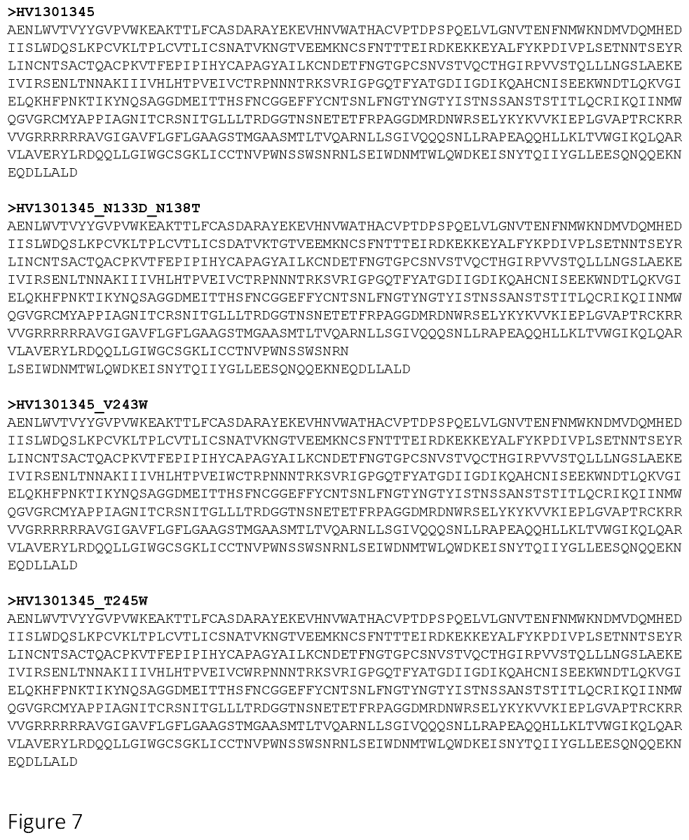

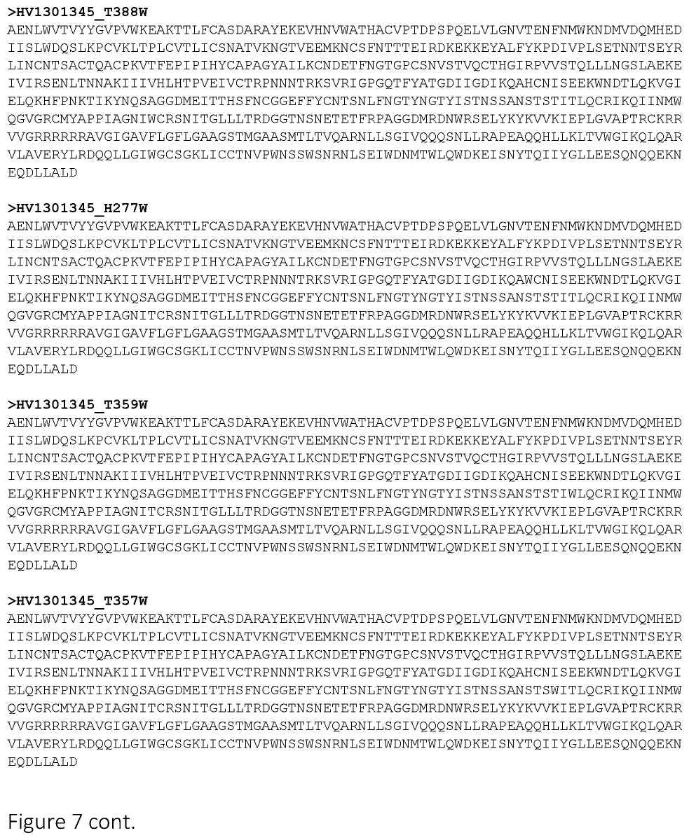

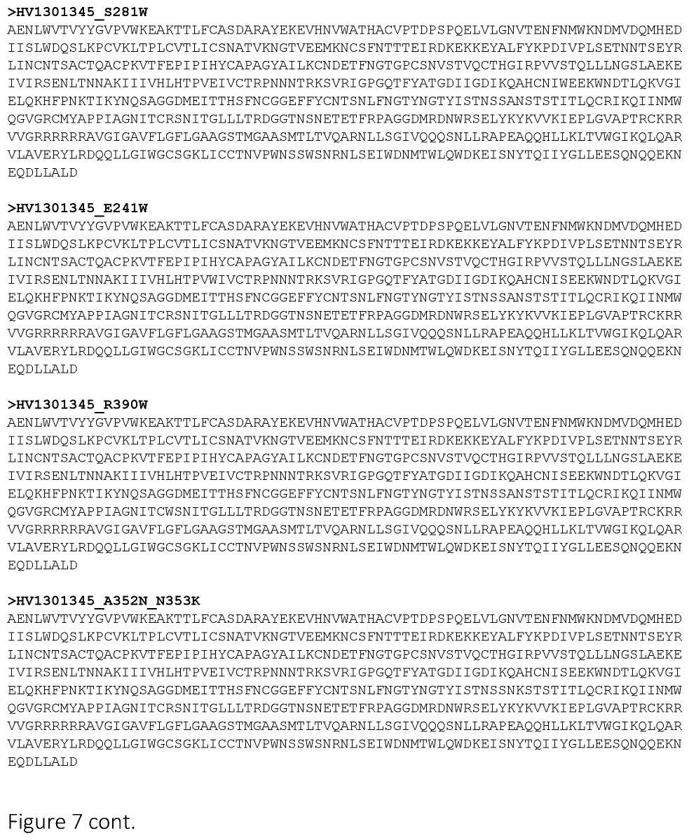

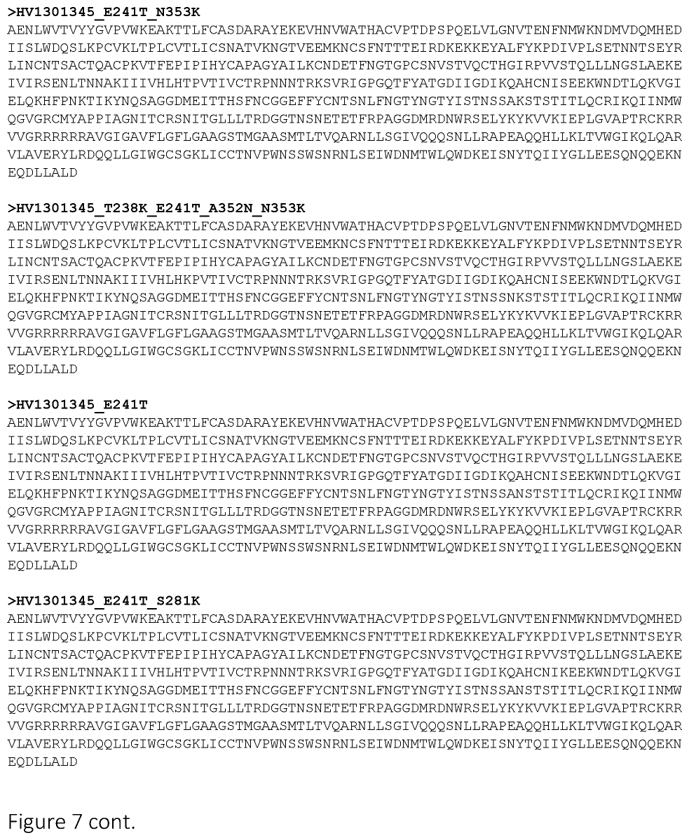

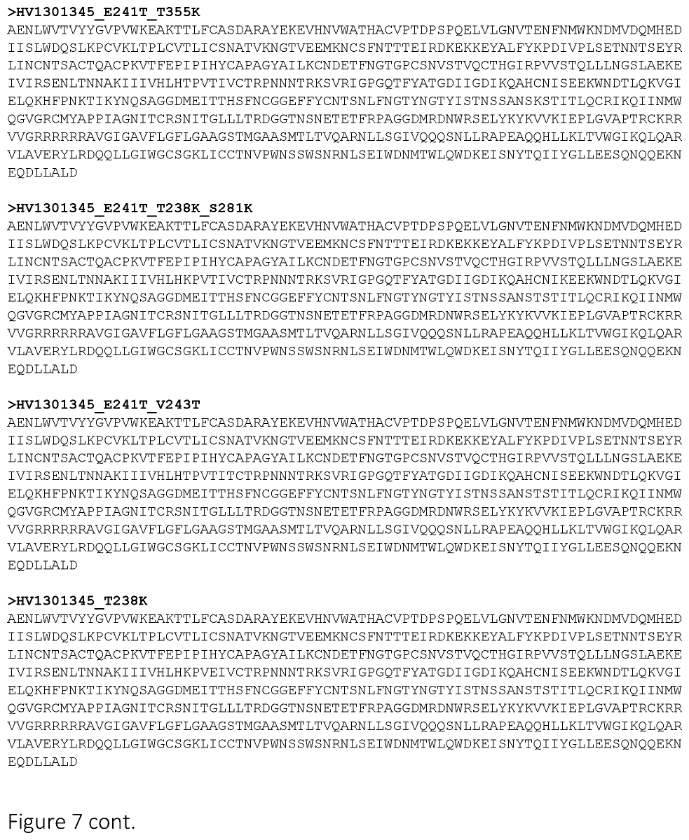

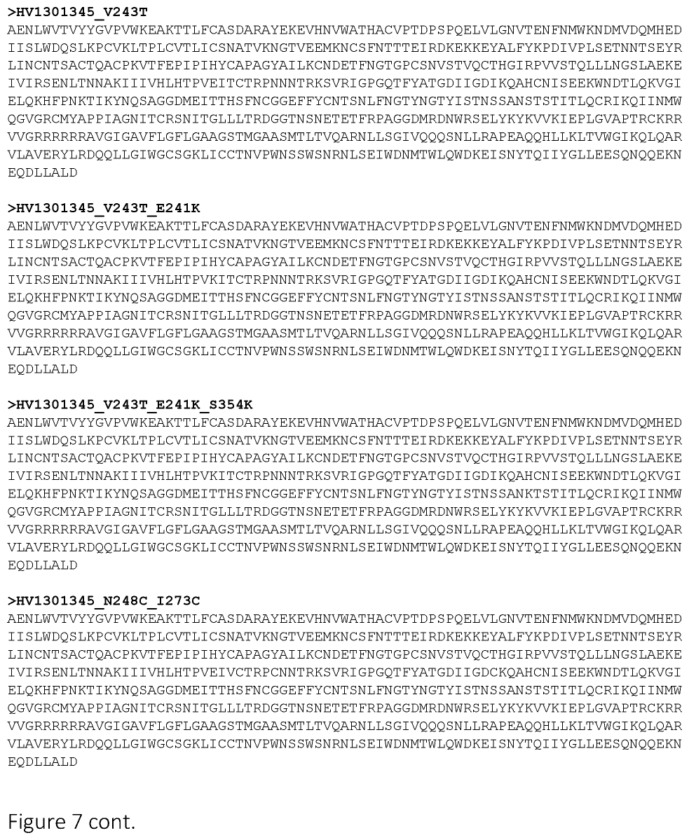

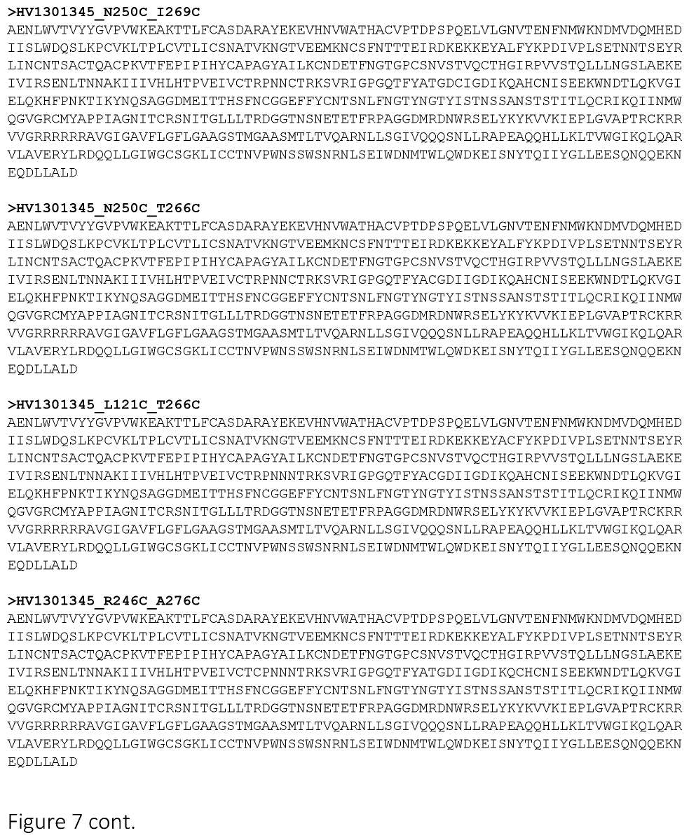

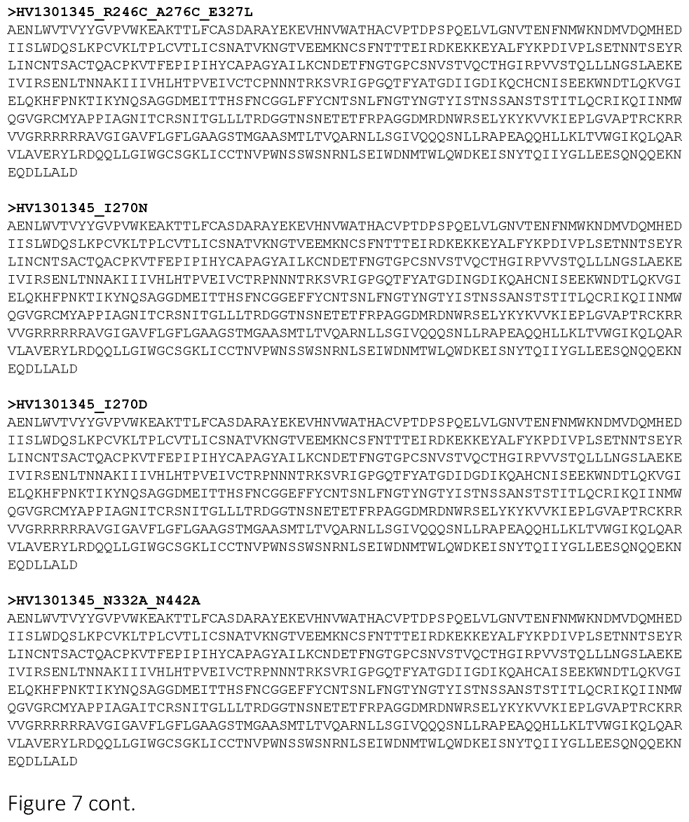

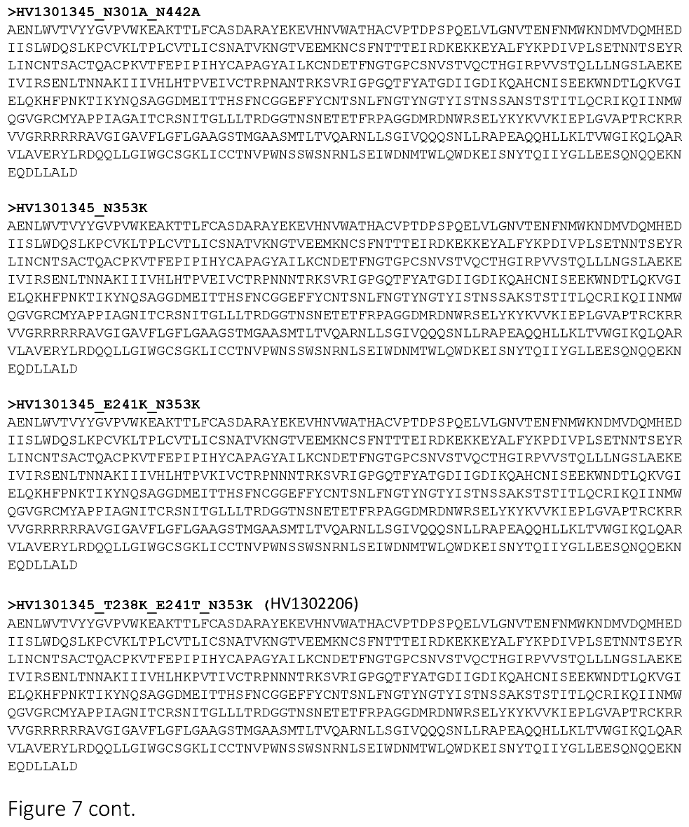

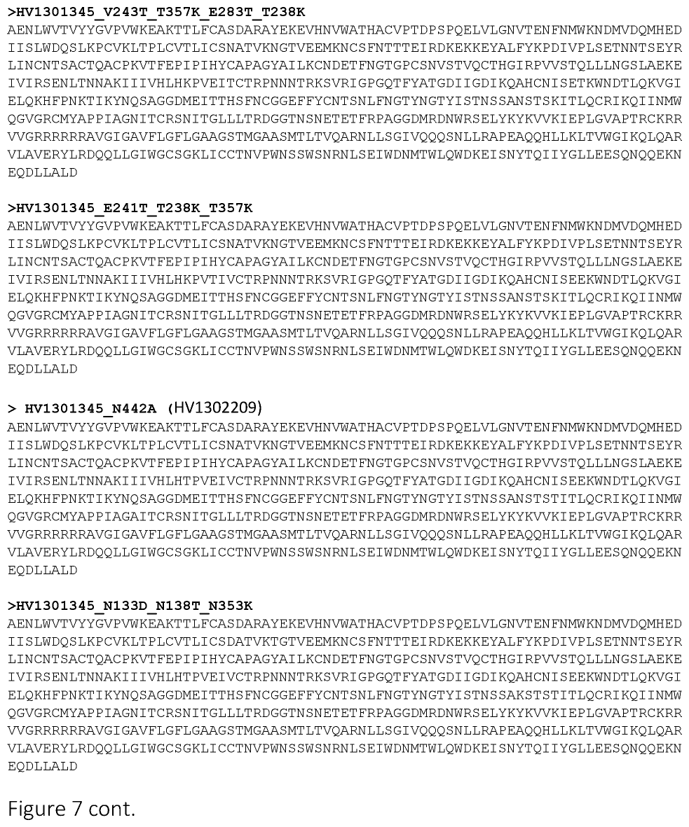

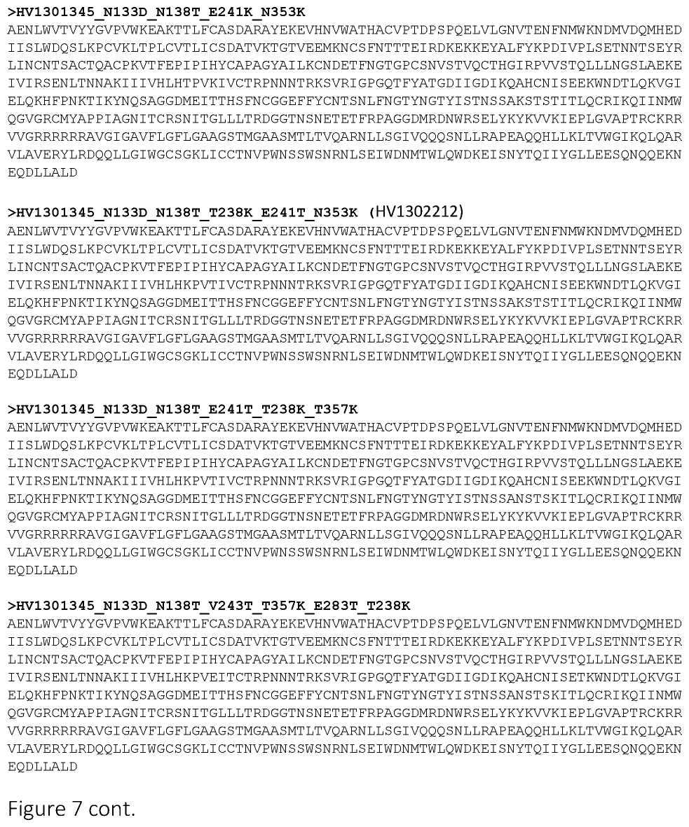

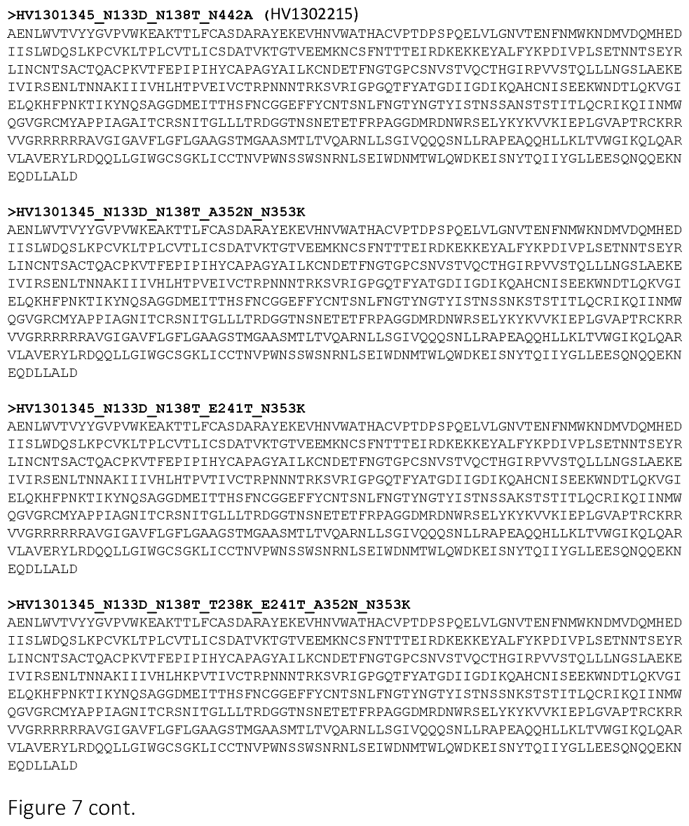

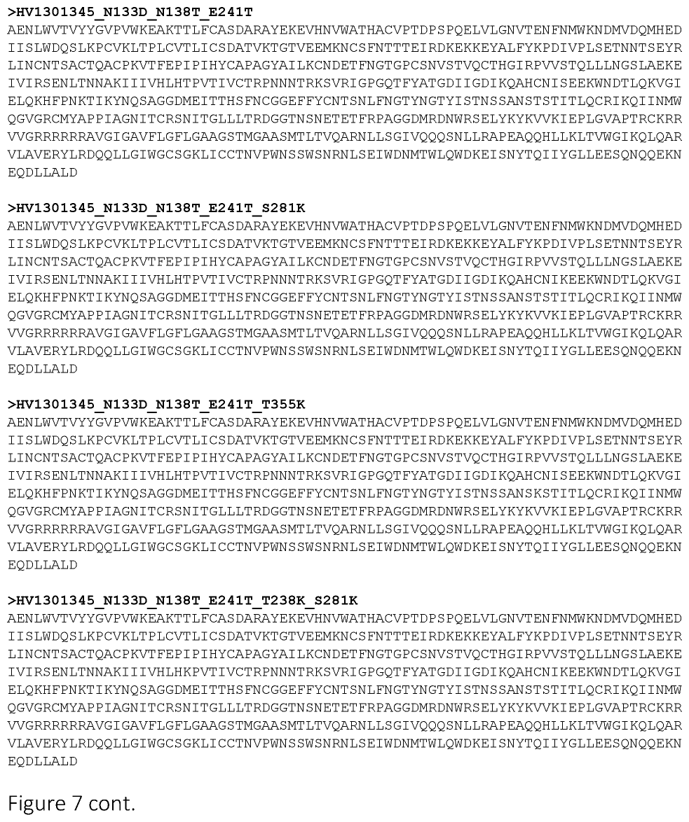

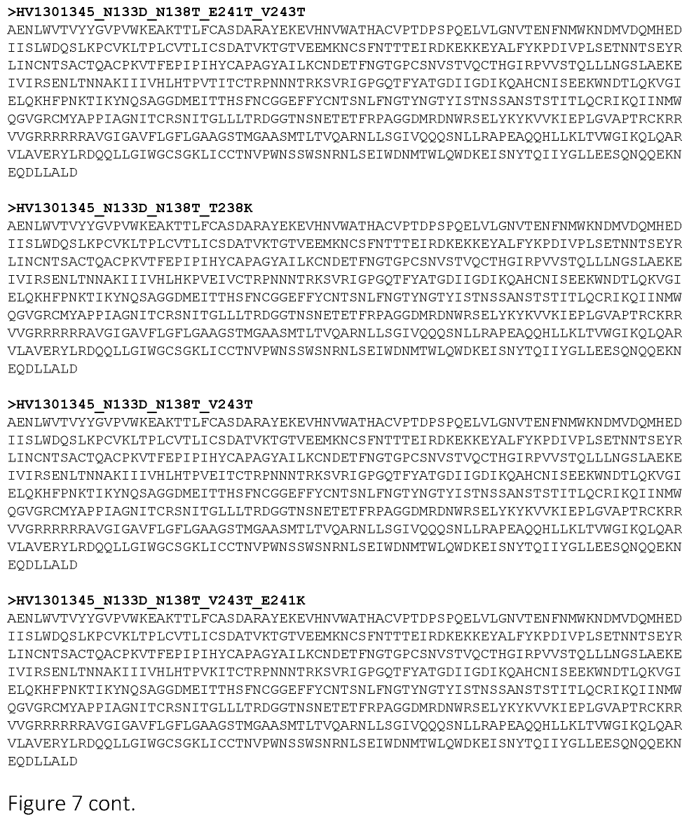

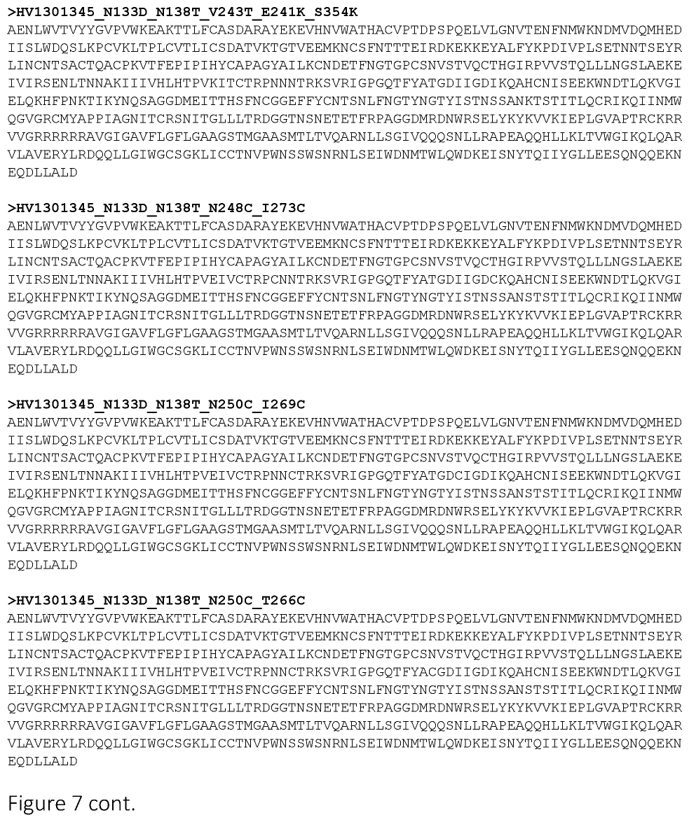

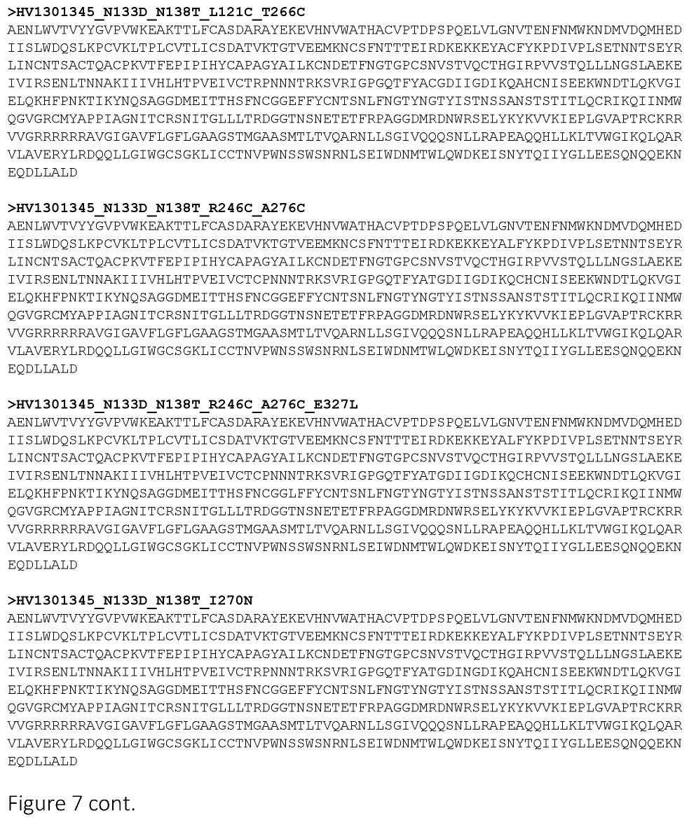

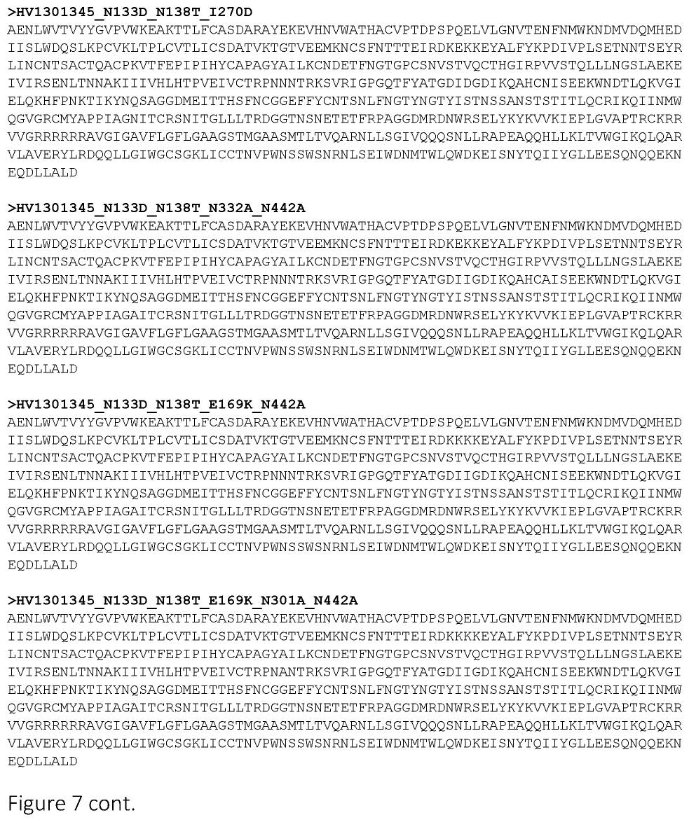

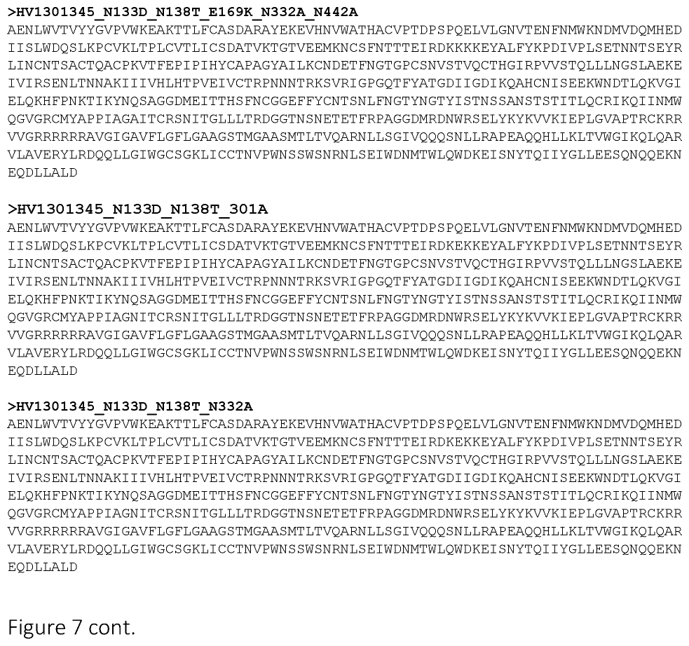

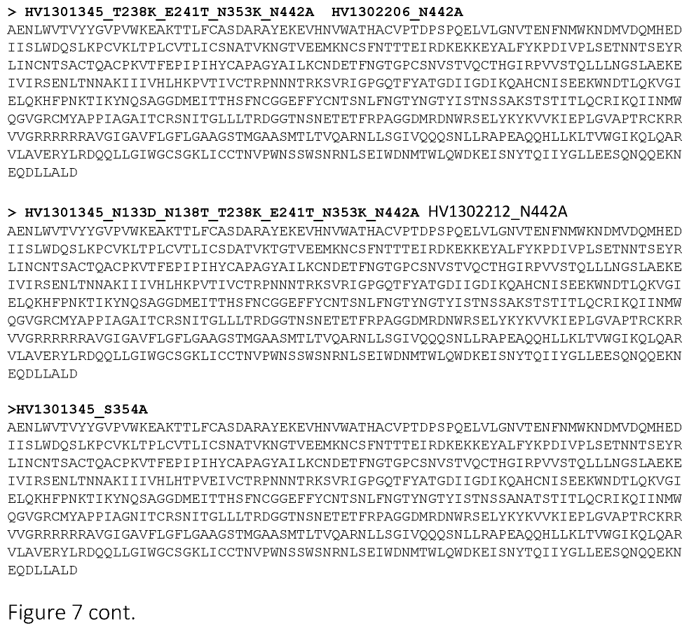

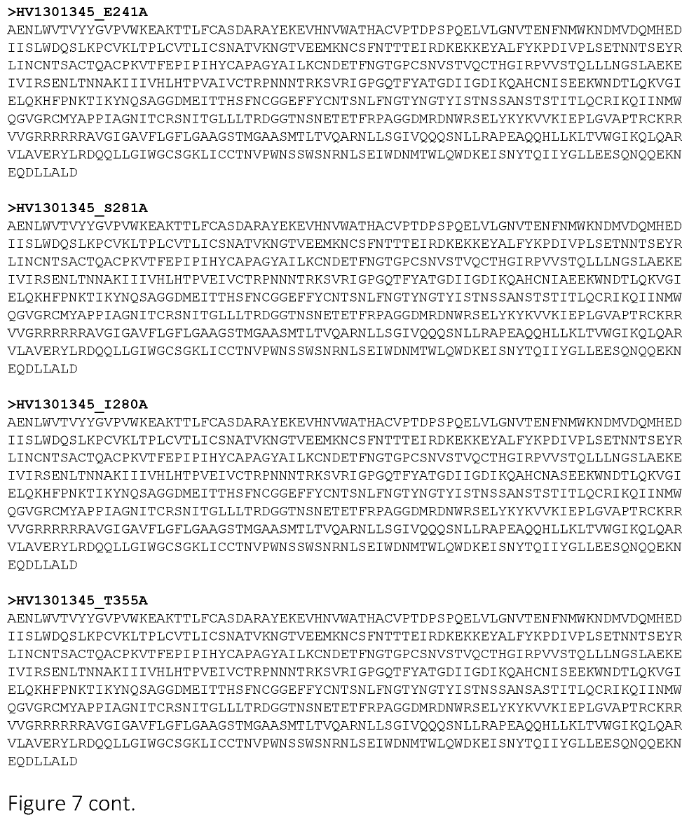

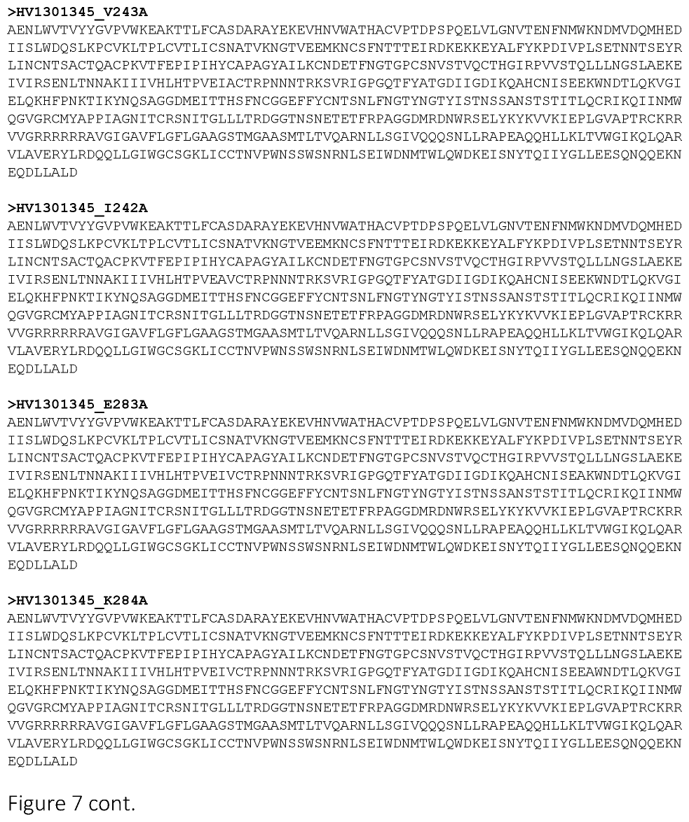

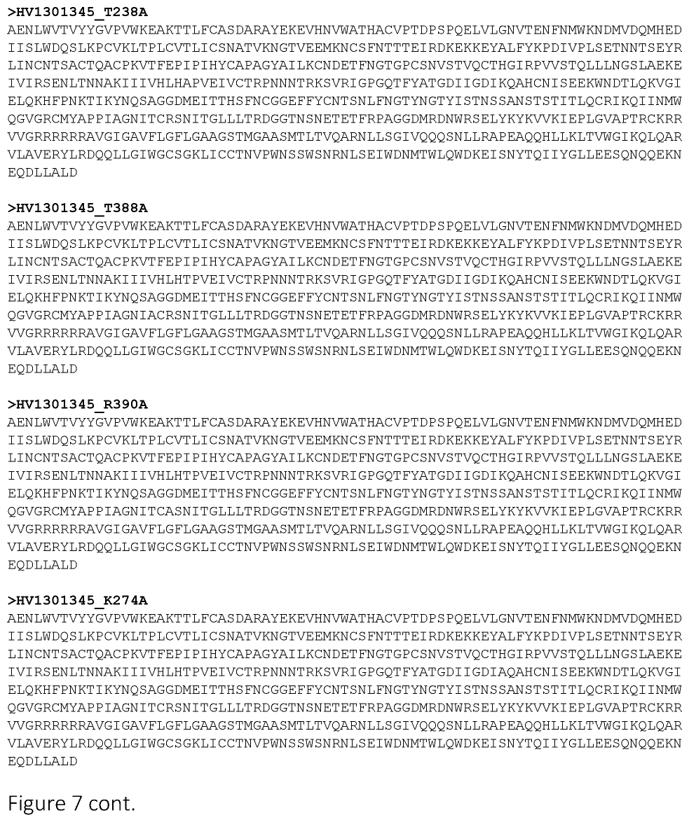

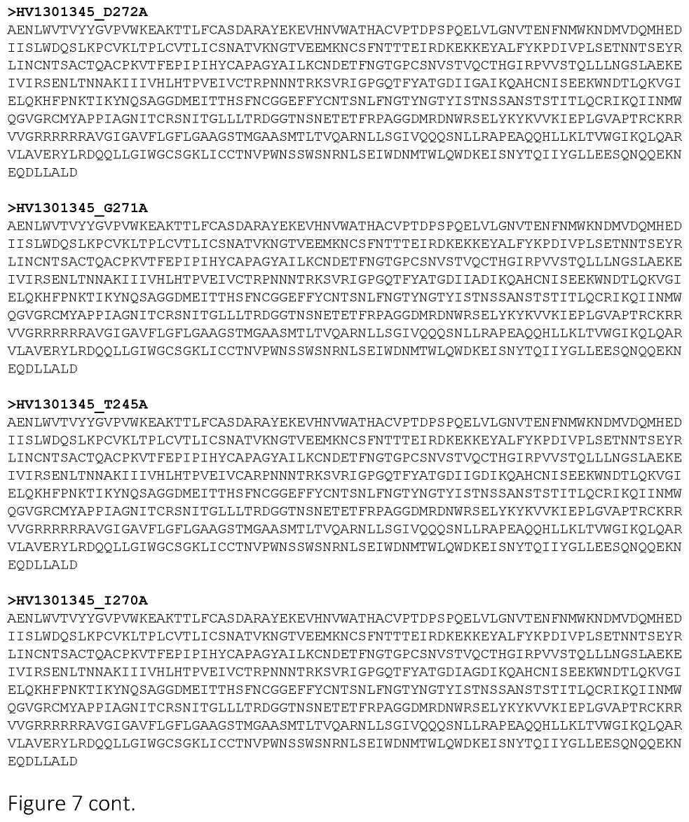

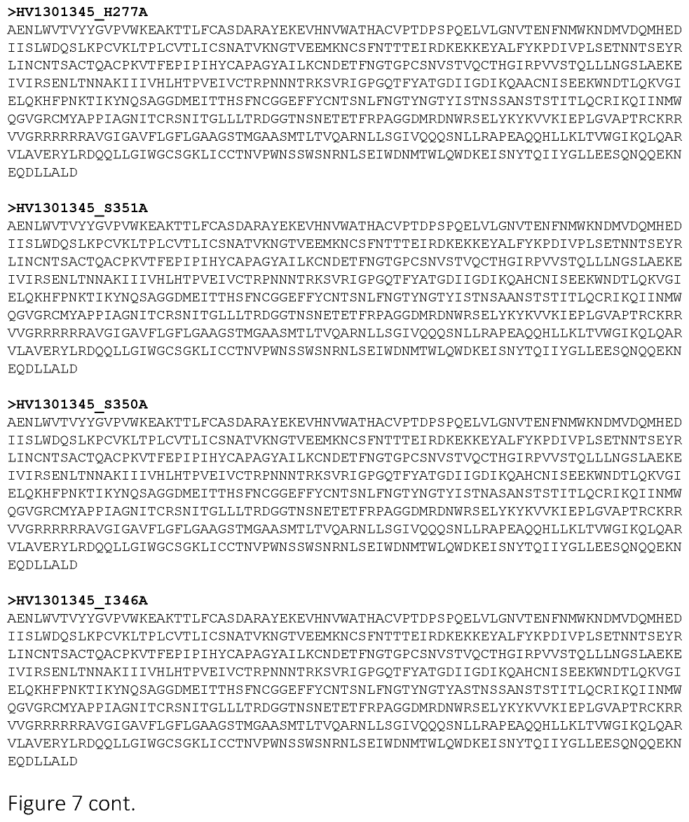

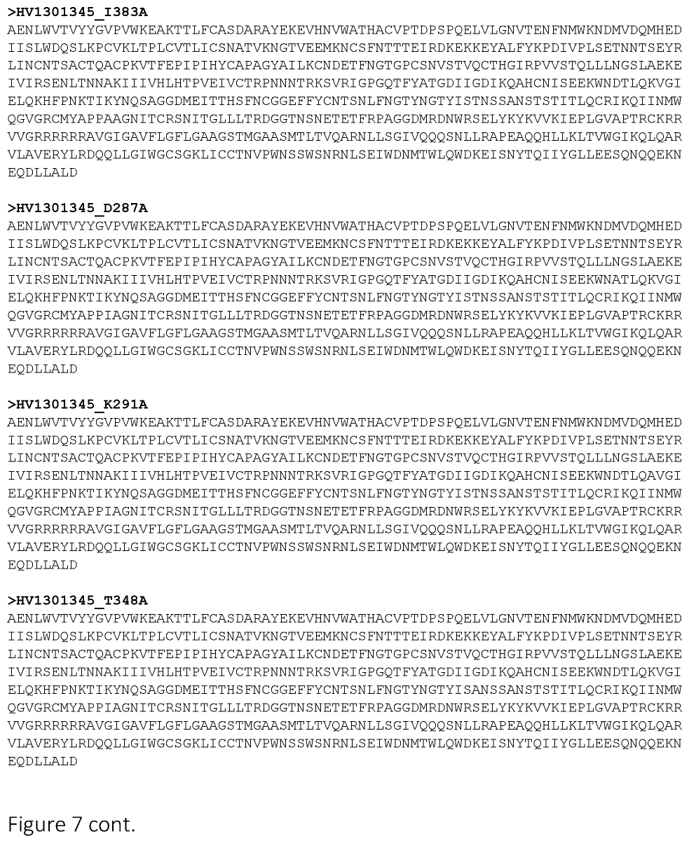

In one embodiment, the nucleic acid is an RNA molecule. In one embodiment, the RNA molecule is transcribed from a DNA sequence described herein. In some embodiments, the RNA molecule is encoded by one of the inventive sequences. In another embodiment, the nucleotide sequence comprises an RNA sequence transcribed by a DNA sequence encoding any one the polypeptide sequences in FIG. 7, or a variant thereof or a fragment thereof. Accordingly, in one embodiment, the invention provides an RNA molecule encoding one or more of inventive envelopes. The RNA can be plus-stranded. Accordingly, in some embodiments, the RNA molecule can be translated by cells without needing any intervening replication steps such as reverse transcription.

In some embodiments, an RNA molecule of the invention can have a 5′ cap (e.g. but not limited to a 7-methylguanosine, 7mG (5′)ppp(5′)NlmpNp). This cap can enhance in vivo translation of the RNA. The 5′ nucleotide of an RNA molecule useful with the invention can have a 5′ triphosphate group. In a capped RNA this can be linked to a 7-methylguanosine via a 5′-to-5′ bridge. In some embodiments, an RNA molecule can have a 3′ poly-A tail. It can also include a poly-A polymerase recognition sequence (e.g. AAUAAA) near its 3′ end. In some embodiments, an RNA molecule useful with the invention can be single-stranded. In some embodiments, an RNA molecule useful with the invention can comprise synthetic RNA.

The recombinant nucleic acid sequence can be an optimized nucleic acid sequence. Such optimization can increase or alter the immunogenicity of the envelope. Optimization can also improve transcription and/or translation. Optimization can include one or more of the following: low GC content leader sequence to increase transcription; mRNA stability and codon optimization; addition of a Kozak sequence (e.g., GCC ACC) for increased translation; addition of an immunoglobulin (Ig) leader sequence encoding a signal peptide; and eliminating to the extent possible cis-acting sequence motifs (i.e., internal TATA boxes).

Methods for in vitro transfection of mRNA and detection of envelope expression are known in the art.

Methods for expression and immunogenicity determination of nucleic acid encoded envelopes are known in the art.

In certain embodiments, the nucleic acid encoding an envelope is operably linked to a promoter inserted an expression vector. In certain aspects, the compositions comprise a suitable carrier. In certain aspects, the compositions comprise a suitable adjuvant.

In certain embodiments, the induced immune response includes induction of antibodies, including but not limited to autologous and/or cross-reactive (broadly) neutralizing antibodies against HIV-1 envelope. Various assays that analyze whether an immunogenic composition induces an immune response, and the type of antibodies induced are known in the art and are also described herein.

In certain aspects, the invention provides an expression vector comprising any of the nucleic acid sequences of the invention, wherein the nucleic acid is operably linked to a promoter. In certain aspects, the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects, the invention provides nucleic acids comprising any one of the nucleic acid sequences of invention. In certain aspects, the invention provides nucleic acids consisting essentially of any one of the nucleic acid sequences of invention. In certain aspects, the invention provides nucleic acids consisting of any one of the nucleic acid sequences of invention. In certain embodiments, the nucleic acid of the invention, is operably linked to a promoter and is inserted in an expression vector. In certain aspects, the invention provides an immunogenic composition comprising the expression vector.

In certain aspects, the invention provides a composition comprising at least one of the nucleic acid sequences of the invention. In certain aspects, the invention provides a composition comprising any one of the nucleic acid sequences of invention. In certain aspects, the invention provides a composition comprising at least one nucleic acid sequence encoding any one of the polypeptides of the invention.

The envelope used in the compositions and methods of the invention can be a gp160, gp150, gp145, gp140, gp120, gp41, N-terminal deletion variants as described herein, cleavage resistant variants as described herein, or codon optimized sequences thereof. In certain embodiments, the composition comprises envelopes as trimers. In certain embodiments, the envelope proteins are multimerized; for example, trimers are attached to a particle such that multiple copies of the trimer are attached and the multimerized envelope is prepared and formulated for immunization in a human. In certain embodiments, the compositions comprise envelopes, including but not limited to trimers as a particulate, high-density array on liposomes or other particles, for example, but not limited to nanoparticles. In some embodiments, the trimers are in a well ordered, near native like or closed conformation. In some embodiments, the trimer compositions comprise a homogenous mix of native like trimers. In some embodiments, the trimer compositions comprise at least 85%, 90%, or 95% native like trimers.

In certain embodiments, the envelope is any of the forms of HIV-1 envelope. In certain embodiments, the envelope is gp120, gp140, gp145 (i.e. with a transmembrane domain), or gp150. In certain embodiments, gp140 designed to form a stable trimer. See Table 1 for non-limiting examples of sequence designs. In certain embodiments, envelope protomers from a trimer which is not a SOSIP timer. In certain embodiments, the trimer is a SOSIP based trimer wherein each protomer comprises additional modifications. In certain embodiments, envelope trimers are recombinantly produced. In certain embodiments, envelope trimers are purified from cellular recombinant fractions by antibody binding and reconstituted in lipid comprising formulations. See for example WO2015/127108 titled “Trimeric HIV-1 envelopes and uses thereof” and WO/2017151801 which contents are herein incorporated by reference in its entirety. In certain embodiments, the envelopes of the invention are engineered and comprise non-naturally occurring modifications.

In certain embodiments, the envelope is in a liposome. In certain embodiments, the envelope comprises a transmembrane domain with a cytoplasmic tail embedded in a liposome. In certain embodiments, the nucleic acid comprises a nucleic acid sequence which encodes a gp120, gp140, gp145, gp150, or gp160.

In certain embodiments, where the nucleic acids are operably linked to a promoter and inserted in a vector, the vector is any suitable vector. Non-limiting examples, include VSV, replicating rAdenovirus type 4, MVA, Chimp adenovirus vectors, pox vectors, and any other vector. In certain embodiments, the nucleic acids are administered in NanoTaxi block polymer nanospheres.

In certain embodiments, the composition and methods comprise an adjuvant. Non-limiting examples include, 3M052, AS01 B, AS01 E, gla/SE, alum, Poly I poly C (poly IC), polyIC/long chain (LC) TLR agonists, TLR7/8 and 9 agonists, or a combination of TLR7/8 and TLR9 agonists (see Moody et al. (2014) J. Virol. March 2014 vol. 88 no. 6 3329-3339), or any other adjuvant. Non-limiting examples of TLR7/8 agonist include TLR7/8 ligands, Gardiquimod, Imiquimod and R848 (resiquimod). A non-limiting embodiment of a combination of TLR7/8 and TLR9 agonist comprises R848 and oCpG in STS (see Moody et al. (2014) J. Virol. March 2014 vol. 88 no. 6 3329-3339). In some embodiments, LNPs can be used as an adjuvant in compositions comprising protein immunogens.

In certain aspects, the invention provides a cell comprising a nucleic acid encoding any one of the envelopes of the invention suitable for recombinant expression. In certain aspects, the invention provides a clonally derived population of cells encoding any one of the envelopes of the invention suitable for recombinant expression. In certain aspects, the invention provides a sable pool of cells encoding any one of the envelopes of the invention suitable for recombinant expression.

In certain aspects, the invention provides a recombinant HIV-1 envelope polypeptide as described herein, wherein the polypeptide is a non-naturally occurring protomer designed to form an envelope trimer. The invention also provides nucleic acids encoding these recombinant polypeptides. Non-limiting examples of amino acids and nucleic acid of such protomers are disclosed herein.

In certain aspects, the invention provides a recombinant trimer comprising three identical protomers of an envelope. In certain aspects the invention provides an immunogenic composition comprising the recombinant trimer and a carrier, wherein the trimer comprises three identical protomers of an HIV-1 envelope as described herein. In certain aspects, the invention provides an immunogenic composition comprising nucleic acid encoding these recombinant HIV-1 envelope and a carrier.

### Sequences/Clones

Described herein are nucleic and amino acids sequences of HIV-1 envelopes. The sequences for use as immunogens are in any suitable form. In certain embodiments, the described HIV-1 envelope sequences are gp160s. In certain embodiments, the described HIV-1 envelope sequences are gp120s. Other sequences, for example but not limited to stable SOSIP trimer designs, gp145s, gp140s, both cleaved and uncleaved, gp140 Envs with the deletion of the cleavage (C) site, fusion (F) and immunodominant (I) region in gp41—named as gp140ΔCFI (gp140CFI), gp140 Envs with the deletion of only the cleavage (C) site and fusion (F) domain—named as gp140ΔCF (gp140CF), gp140 Envs with the deletion of only the cleavage (C)—named gp140ΔC (gp140C) (See e.g. Liao et al. Virology 2006, 353, 268-282), gp150s, gp41s, which are readily derived from the nucleic acid and amino acid gp160 sequences. In certain embodiments, the nucleic acid sequences are codon optimized for optimal expression in a host cell, for example a mammalian cell, a rBCG cell or any other suitable expression system.

An HIV-1 envelope has various structurally defined fragments/forms: gp160; gp140—including cleaved gp140 and uncleaved gp140 (gp140C), gp140CF, or gp140CFI; gp120 and gp41. A skilled artisan appreciates that these fragments/forms are defined not necessarily by their crystal structure, but by their design and bounds within the full length of the gp160 envelope. While the specific consecutive amino acid sequences of envelopes from different strains are different, the bounds and design of these forms are well known and characterized in the art.

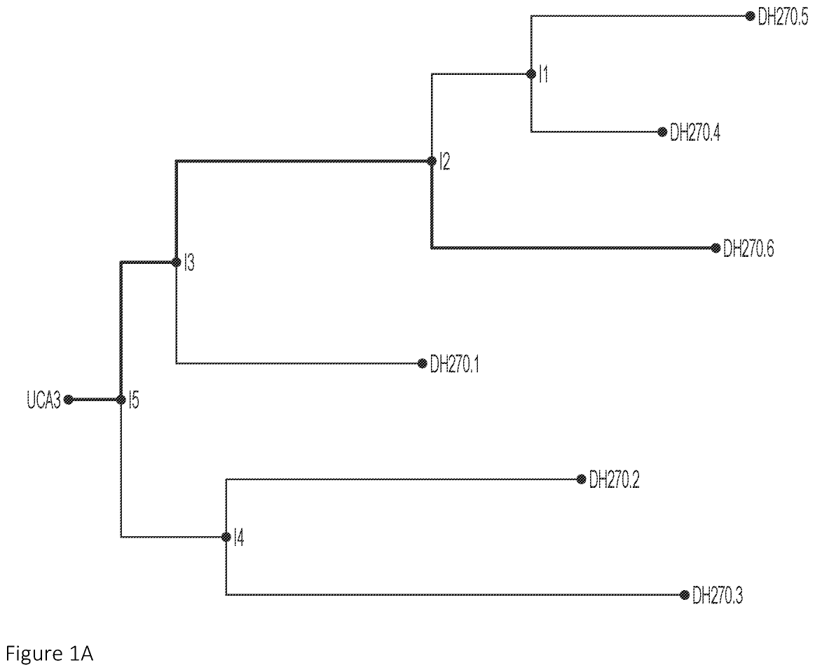

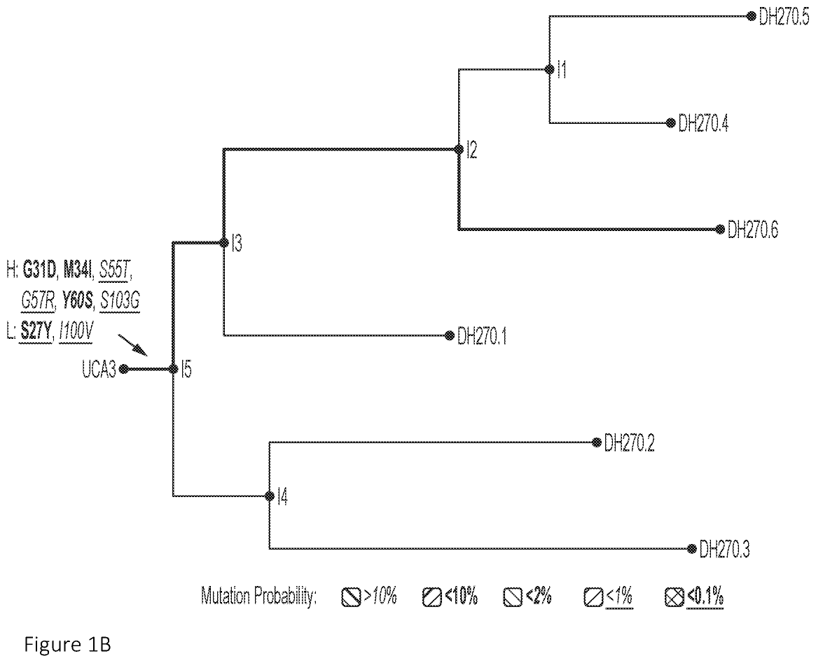

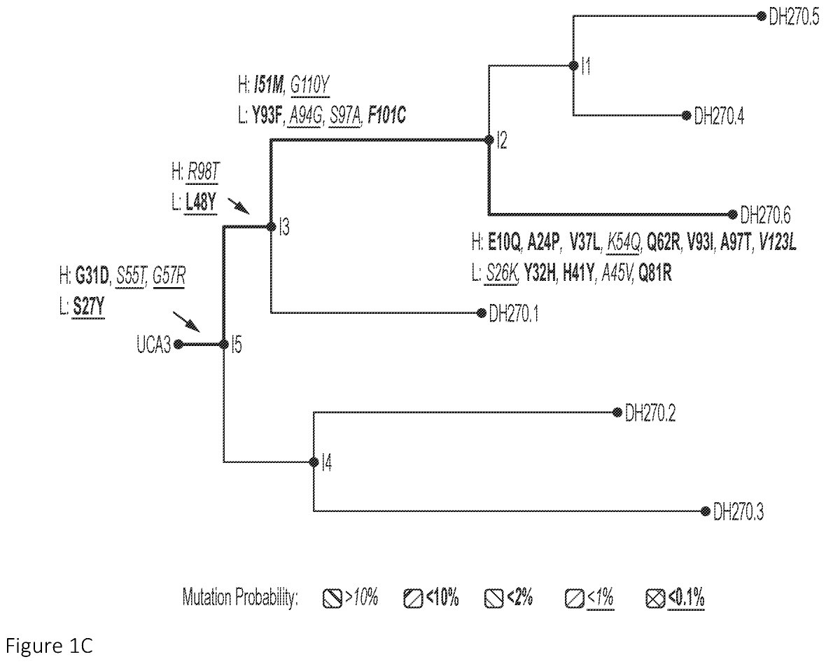

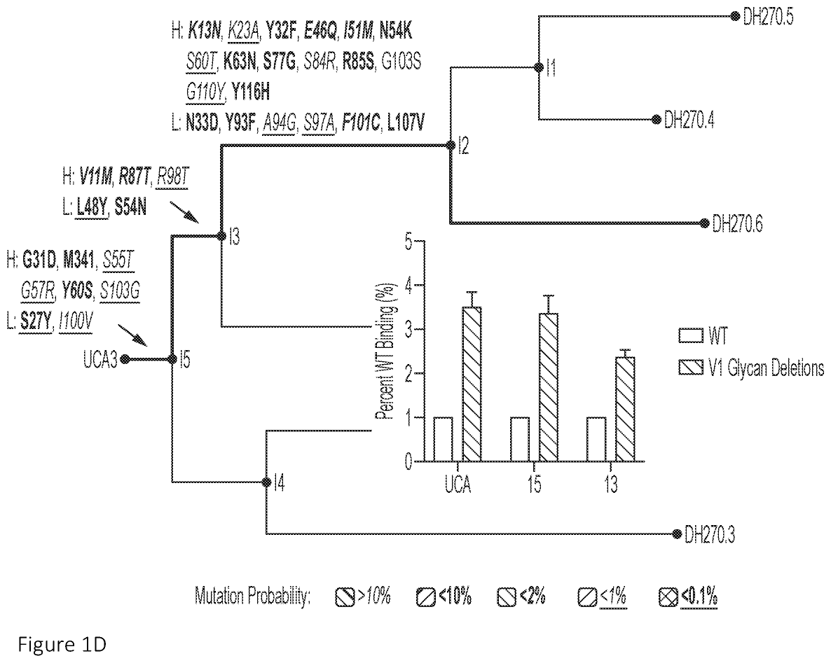

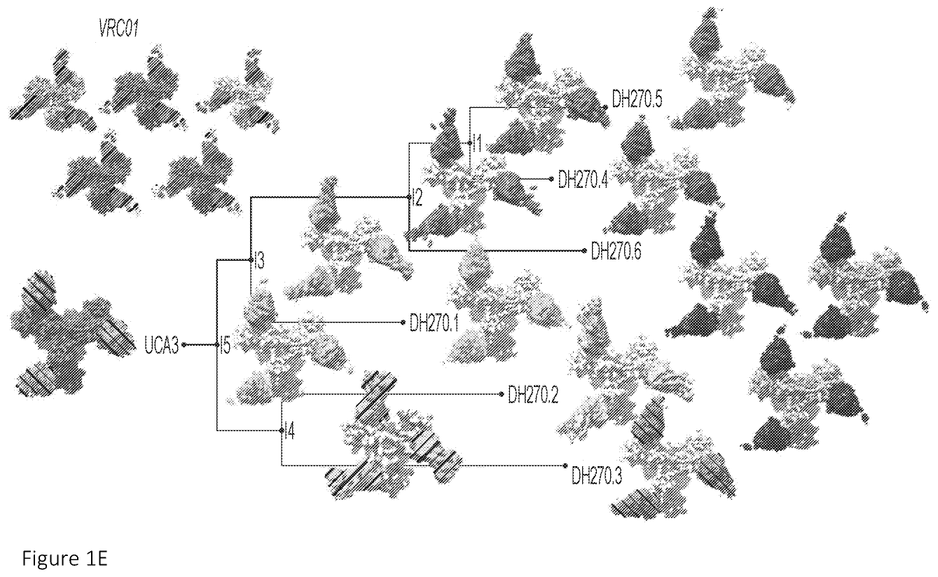

For example, it is well known in the art that during its transport to the cell surface, the gp160 polypeptide is processed and proteolytically cleaved to gp120 and gp41 proteins. Cleavages of gp160 to gp120 and gp41 occurs at a conserved cleavage site “REKR.” See Chakrabarti et al. Journal of Virology vol. 76, pp. 5357-5368 (2002) see for example FIG. 1, and Second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol. 353(2): 268-282 (2006).

The role of the furin cleavage site was well understood both in terms of improving cleave efficiency, see Binley et al. supra, and eliminating cleavage, see Bosch and Pawlita, Virology 64 (5):2337-2344 (1990); Guo et al. Virology 174: 217-224 (1990); McCune et al. Cell 53:55-67 (1988); Liao et al. J Virol. April; 87(8):4185-201 (2013).

Likewise, the design of gp140 envelope forms is also well known in the art, along with the various specific changes which give rise to the gp140C (uncleaved envelope), gp140CF and gp140CFI forms. Envelope gp140 forms are designed by introducing a stop codon within the gp41 sequence. See Chakrabarti et al. at FIG. 1.

Envelope gp140C refers to a gp140 HIV-1 envelope design with a functional deletion of the cleavage (C) site, so that the gp140 envelope is not cleaved at the furin cleavage site. The specification describes cleaved and uncleaved forms, and various furin cleavage site modifications that prevent envelope cleavage are known in the art. In some embodiments of the gp140C form, two of the R residues in and near the furin cleavage site are changed to E; e.g., RRVVEREKR is changed to ERVVEREKE, and is one example of an uncleaved gp140 form. Another example is the gp140C form which has the REKR site changed to SEKS. See supra for references.

Envelope gp140CF refers to a gp140 HIV-1 envelope design with a deletion of the cleavage (C) site and fusion (F) region. Envelope gp140CFI refers to a gp140 HIV-1 envelope design with a deletion of the cleavage (C) site, fusion (F) and immunodominant (I) region in gp41. See Chakrabarti et al. Journal of Virology vol. 76, pp. 5357-5368 (2002) see for example FIG. 1, and Second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol. 353(2): 268-282 (2006).

In certain embodiments, the envelope design in accordance with this invention involves deletion of residues (e.g., 5-11, 5, 6, 7, 8, 9, 10, or 11 amino acids) at the N-terminus. For delta N-terminal design, amino acid residues ranging from 4 residues or even fewer to 14 residues or even more are deleted. These residues are between the maturation (signal peptide, such as ending with CXX, X can be any amino acid) and “VPVXXXX . . . “. In case of CH505 T/F Env as an example, 8 amino acids (italicized and underlined in the below sequence) were deleted: MRVMGIQRNYPQWWIWSMLGFWMLMICNGMWVTVYYGVPVWKEAKTTLFCASDAKA YEKEVHNVWATHACVPTDPNPQE . . . (rest of envelope sequence is indicated as” . . . ”). In other embodiments, the delta N-design described for CH505 T/F envelope can be used to make delta N-designs of other envelopes. In certain embodiments, the invention provides an immunogen, gp160, gp120 or gp140, without an N-terminal Herpes Simplex gD tag substituted for amino acids of the N-terminus of gp120, with an HIV leader sequence (or other leader sequence), and without the original about 4 to about 25, for example 4, 5, 6, 7, 8, 9, 10, 11, 12, 13, 14, 15, 16, 17, 18, 19, 20, 21, 22, 23, 24, 25 amino acids of the N-terminus of the envelope (e.g. gp120). See WO2013/006688, e.g. at pages 10-12, the contents of which publication is hereby incorporated by reference in its entirety.

The general strategy of deletion of N-terminal amino acids of envelopes results in proteins; for example, gpl20s expressed in mammalian cells that are primarily monomeric, as opposed to dimeric, and, therefore, solves the production and scalability problem of commercial gp120 Env vaccine production. In other embodiments, the amino acid deletions at the N-terminus result in increased immunogenicity of the envelopes.

In certain aspects, the invention provides composition and methods which use a selection of Envs, as gp120s, gp140s cleaved and uncleaved, gp145s, gp150s and gp160s, stabilized and/or multimerized trimers, as proteins, DNAs, RNAs, or any combination thereof, administered as primes and boosts to elicit an immune response. Envs as proteins can be co-administered with nucleic acid vectors containing Envs to amplify antibody induction. In certain embodiments, the compositions and methods include any immunogenic HIV-1 sequences to give the best coverage for T cell help and cytotoxic T cell induction. In certain embodiments, the compositions and methods include mosaic and/or consensus HIV-1 genes to give the best coverage for T cell help and cytotoxic T cell induction. In certain embodiments, the compositions and methods include mosaic group M and/or consensus genes to give the best coverage for T cell help and cytotoxic T cell induction. In some embodiments, the mosaic genes are any suitable gene from the HIV-1 genome. In some embodiments, the mosaic genes are Env genes, Gag genes, Pol genes, Nef genes, or any combination thereof. See e.g. U.S. Pat. No. 7,951,377. In some embodiments, the mosaic genes are bivalent mosaics. In some embodiments, the mosaic genes are trivalent. In some embodiments, the mosaic genes are administered in a suitable vector with each immunization with Env gene inserts in a suitable vector and/or as a protein. In some embodiments, the mosaic genes, for example as bivalent mosaic Gag group M consensus genes, are administered in a suitable vector; for example but not limited to HSV2, can be administered with each immunization with Env gene inserts in a suitable vector, for example but not limited to HSV-2.

In certain aspects, the invention provides compositions and methods of Env genetic immunization, such as alone or with Env proteins to recreate the swarms of evolved viruses that have led to bnAb induction. Nucleotide-based vaccines offer a flexible vector format to immunize against virtually any protein antigen. Currently, two types of genetic vaccination are available for testing-DNAs and mRNAs.

In certain aspects the invention provides using immunogenic compositions wherein immunogens are delivered as DNA. See Graham B S, Enama M E, Nason M C, Gordon I J, Peel S A, et al. (2013) DNA Vaccine Delivered by a Needle-Free Injection Device Improves Potency of Priming for Antibody and CD8+ T-Cell Responses after rAd5 Boost in a Randomized Clinical Trial. PLOS ONE 8(4): e59340, page 9. Various technologies for delivery of nucleic acids, as DNA and/or RNA, so as to elicit immune response, both T-cell and humoral responses, are known in the art and are under developments. In certain embodiments, DNA can be delivered as naked DNA. In certain embodiments, DNA is formulated for delivery by a gene gun. In certain embodiments, DNA is administered by electroporation, or by a needle-free injection technologies, for example but not limited to Biojector® device. In certain embodiments, the DNA is inserted in vectors. The DNA is delivered using a suitable vector for expression in mammalian cells. In certain embodiments, the nucleic acids encoding the envelopes are optimized for expression. In certain embodiments, DNA is optimized, e.g. codon optimized, for expression. In certain embodiments, the nucleic acids are optimized for expression in vectors and/or in mammalian cells. In non-limiting embodiments, these are bacterially derived vectors, adenovirus based vectors, rAdenovirus (e.g. Barouch D H, et al. Nature Med. 16: 319-23, 2010), recombinant mycobacteria (e.g. rBCG or M smegmatis) (Yu, J S et al. Clinical Vaccine Immunol. 14: 886-093,2007; ibid 13: 1204-11,2006), and recombinant vaccinia type of vectors (Santra S. Nature Med. 16: 324-8, 2010), for example but not limited to ALVAC, replicating (Kibler K V et al., PLOS One 6: e25674, 2011 Nov. 9) and non-replicating (Perreau M et al. J. virology 85: 9854-62, 2011) NYVAC, modified vaccinia Ankara (MVA)), adeno-associated virus, Venezuelan equine encephalitis (VEE) replicons, Herpes Simplex Virus vectors, and other suitable vectors.

In certain aspects, the invention provides using immunogenic compositions wherein immunogens are delivered as DNA or RNA in suitable formulations. Various technologies which encompass using DNA or RNA, or can use complexes of nucleic acid molecules and other entities to be used in immunization. In certain embodiments, DNA or RNA is administered as nanoparticles consisting of low dose antigen-encoding DNA formulated with a block copolymer (amphiphilic block copolymer 704). See Cany et al., Journal of Hepatology 2011 vol. 54 j 115-121; Arnaoty et al., Chapter 17 in Yves Bigot (ed.), Mobile Genetic Elements: Protocols and Genomic Applications, Methods in Molecular Biology, vol. 859, pp293-305 (2012); Arnaoty et al. (2013) Mol Genet Genomics. 2013 August; 288(7-8):347-63. Nanocarrier technologies called Nanotaxi® for immunogenic macromolecules (DNA, RNA, Protein) delivery are under development. See for example technologies developed by Incellart.

In certain aspects, the invention provides using immunogenic compositions wherein immunogens are delivered as recombinant proteins. Various methods for production and purification of recombinant proteins, including trimers such as but not limited to SOSIP based trimers, suitable for use in immunization are known in the art. In certain embodiments, recombinant proteins are produced in CHO cells.

It is readily understood that the envelope glycoproteins referenced in various examples and figures comprise a signal/leader sequence. It is well known in the art that HIV-1 envelope glycoprotein is a secretory protein with a signal or leader peptide sequence that is removed during processing and recombinant expression (without removal of the signal peptide, the protein is not secreted). See for example Li et al. Control of expression, glycosylation, and secretion of HIV-1 gp120 by homologous and heterologous signal sequences. Virology 204(1):266-78 (1994) (“Li et al. 1994”), at first paragraph, and Li et al. Effects of inefficient cleavage of the signal sequence of HIV-1 gp120 on its association with calnexin, folding, and intracellular transport. PNAS 93:9606-9611 (1996) (“Li et al. 1996”), at 9609. Any suitable signal sequence can be used. In some embodiments, the leader sequence is the endogenous leader sequence. Most of the gp120 and gp160 amino acid sequences include the endogenous leader sequence. In other non-limiting examples, the leader sequence is human Tissue Plasminogen Activator (TPA) sequence or human CD5 leader sequence (e.g. MPMGSLQPLATLYLLGMLVASVLA). Most of the chimeric designs include CD5 leader sequence. A skilled artisan appreciates that when used as immunogens, and for example when recombinantly produced, the amino acid sequences of the recombinant proteins do not comprise the leader peptide sequences.

The immunogenic envelopes can also be administered as a protein prime and/or boost alone or in combination with a variety of nucleic acid envelope primes (e.g., HIV-1 Envs delivered as DNA expressed in viral or bacterial vectors).

Dosing of proteins and nucleic acids can be readily determined by a skilled artisan. A single dose of nucleic acid can range from a few nanograms (ng) to a few micrograms (μg) or milligram of a single immunogenic nucleic acid. Recombinant protein dose can range from a few μg micrograms to a few hundred micrograms, or milligrams of a single immunogenic polypeptide.

Administration: The compositions can be formulated with appropriate carriers using known techniques to yield compositions suitable for various routes of administration. In certain embodiments, the compositions are delivered via intramascular (IM), via subcutaneous, via intravenous, via nasal, via mucosal routes, or any other suitable route of immunization.

The compositions can be formulated with appropriate carriers and adjuvants using techniques to yield compositions suitable for immunization. The compositions can include an adjuvant, such as, for example but not limited to 3M052 in any suitable formulation, alum, poly IC, MF-59 or other squalene-based adjuvant, ASOIB, or other liposomal based adjuvant suitable for protein or nucleic acid immunization. In certain embodiments, the adjuvant is GSK AS01E adjuvant containing MPL and QS21. This adjuvant has been shown by GSK to be as potent as the similar adjuvant AS01B but to be less reactogenic using HBsAg as vaccine antigen (Leroux-Roels et al., IABS Conference, April 2013). In certain embodiments, TLR agonists are used as adjuvants. In other embodiments, adjuvants which break immune tolerance are included in the immunogenic compositions.

In certain embodiments, the compositions and methods comprise any suitable agent or immune modulation which can modulate mechanisms of host immune tolerance and release of the induced antibodies. In non-limiting embodiments, modulation includes PD-1 blockade; T regulatory cell depletion; CD40L hyperstimulation; soluble antigen administration, wherein the soluble antigen is designed such that the soluble agent eliminates B cells targeting dominant epitopes, or a combination thereof. In certain embodiments, an immunomodulatory agent is administered at a time and in an amount sufficient for transient modulation of the subject's immune response so as to induce an immune response which comprises broad neutralizing antibodies against HIV-1 envelope. Non-limiting examples of such agents is any one of the agents described herein: e.g. chloroquine (CQ), PTPIB Inhibitor—CAS 765317-72-4—Calbiochem or MSI 1436 clodronate or any other bisphosphonate; a Foxol inhibitor, e.g. 344355|Foxol Inhibitor, AS1842856—Calbiochem; Gleevac, anti-CD25 antibody, anti-CCR4 Ab, an agent which binds to a B cell receptor for a dominant HIV-1 envelope epitope, or any combination thereof. In non-limiting embodiments, the modulation includes administering an anti-CTLA4 antibody, OX-40 agonists, or a combination thereof. Non-limiting examples are of CTLA-1 antibody are ipilimumab and tremelimumab. In certain embodiments, the methods comprise administering a second immunomodulatory agent, wherein the second and first immunomodulatory agents are different.

### Multimeric Envelopes

Presentation of antigens as particulates reduces the B cell receptor affinity necessary for signal transduction and expansion (see Baptista et al. EMBO J. 2000 Feb. 15; 19(4): 513-520). Displaying multiple copies of the antigen on a particle provides an avidity effect that can overcome the low affinity between the antigen and B cell receptor. The initial B cell receptor specific for pathogens can be low affinity, which precludes vaccines from being able to stimulate and expand B cells of interest. Very few naïve B cells from which HIV-1 broadly neutralizing antibodies arise can bind to soluble HIV-1 Envelope. Provided are envelopes, including but not limited to trimers as particulate, high-density array on liposomes or other particles, for example but not limited to nanoparticles. See e.g. He et al. Nature Communications 7, Article number: 12041 (2016), doi:10.1038/ncomms12041; Bamrungsap et al. Nanomedicine, 2012, 7 (8), 1253-1271.

To improve the interaction between the naïve B cell receptor and immunogens, envelope designed can be created to wherein the envelope is presented on particles, e.g. but not limited to nanoparticle. In some embodiments, the HIV-1 Envelope trimer can be fused to ferritin. Ferritin protein self assembles into a small nanoparticle with three fold axis of symmetry. At these axes the envelope protein is fused. Therefore, the assembly of the three-fold axis also clusters three HIV-1 envelope protomers together to form an envelope trimer. Each ferritin particle has 8 axes which equates to 8 trimers being displayed per particle. See e.g. Sliepen et al. Retrovirology 2015 12:82, DOI: 10.1186/s12977-015-0210-4.

Any suitable ferritin sequence can be used. In non-limiting embodiments, ferritin sequences are disclosed in WO/2018/005558. Two-chain ferritin sequences are also provided for use in making ferritin nanoparticles.

Ferritin nanoparticle linkers: The ability to form HIV-1 envelope ferritin nanoparticles relies self-assembly of 24 ferritin subunits into a single ferritin nanoparticle. The addition of a ferritin subunit to the c-terminus of HIV-1 envelope can interfere with the ability of the ferritin subunit to fold properly and or associate with other ferritin subunits. When expressed alone ferritin readily forms 24-subunit nanoparticles, however appending it to envelope only yields nanoparticles for certain envelopes. Since the ferritin nanoparticle forms in the absence of envelope, the envelope can be sterically hindering the association of ferritin subunits. Thus, ferritin can be designed with elongated glycine-serine linkers to further distance the envelope from the ferritin subunit. To make sure that the glycine linker is attached to ferritin at the correct position, constructs can be created that attach at second amino acid position or the fifth amino acid position. The first four n-terminal amino acids of natural Helicobacter pylori ferritin are not needed for nanoparticle formation but can be critical for proper folding and oligomerization when appended to envelope. Thus, constructs can be designed with and without the leucine, serine, and lysine amino acids following the glycine-serine linker. The goal will be to find a linker length that is suitable for formation of envelope nanoparticles when ferritin is appended to most envelopes. Any suitable linker between the envelope and ferritin can be used, so long as the fusion protein is expressed and the trimer is formed.

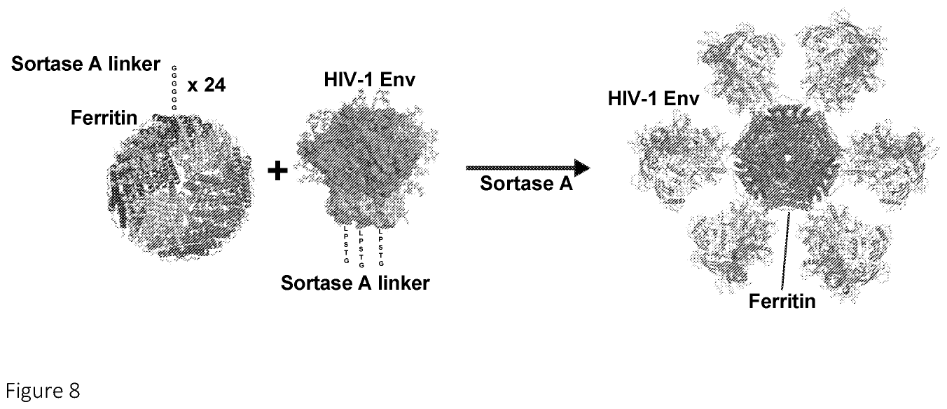

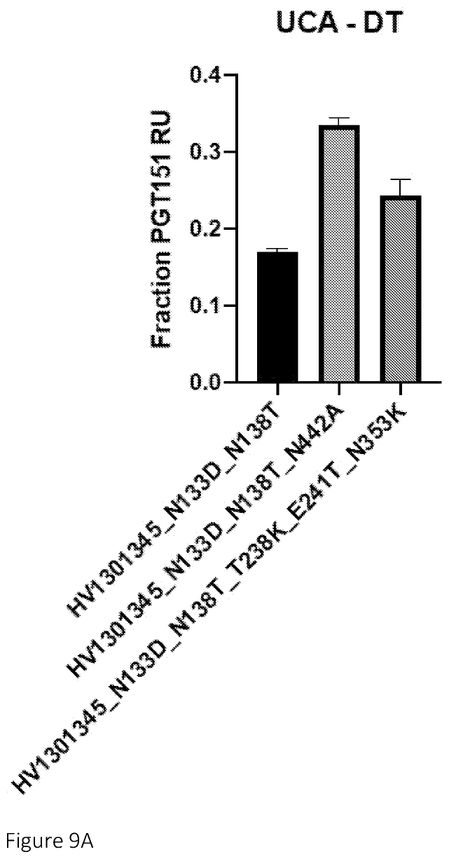

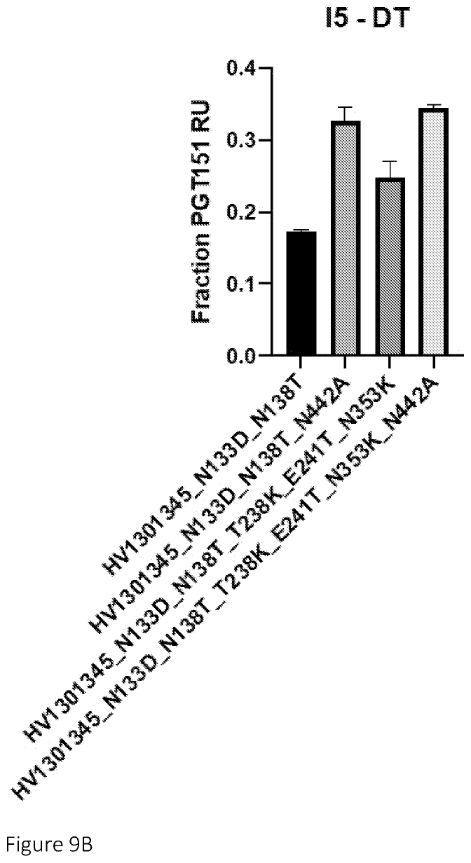

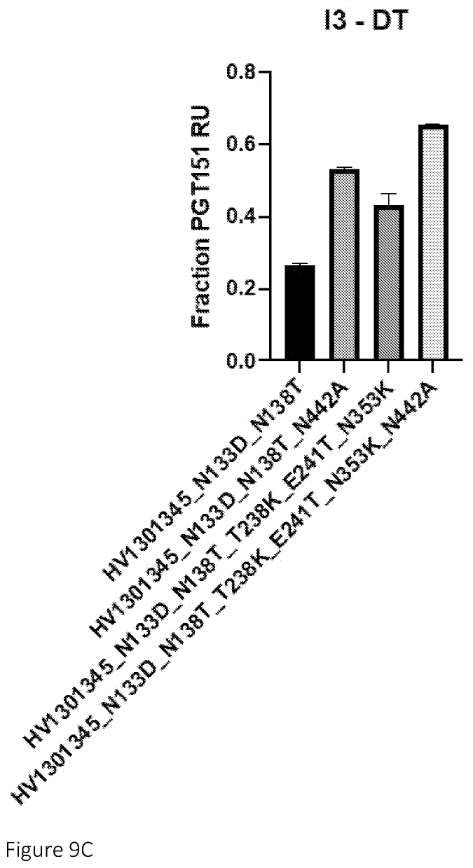

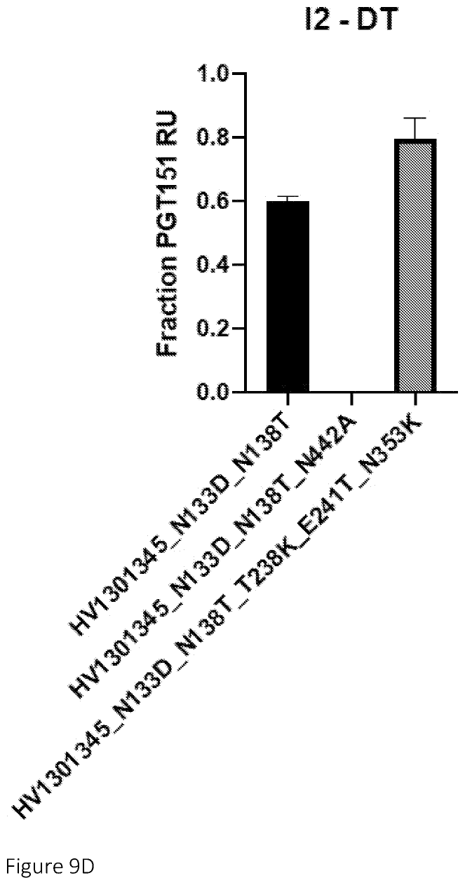

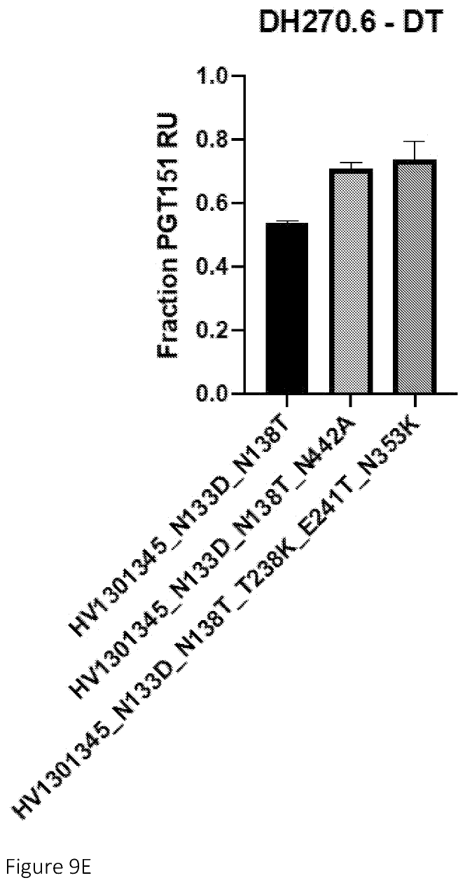

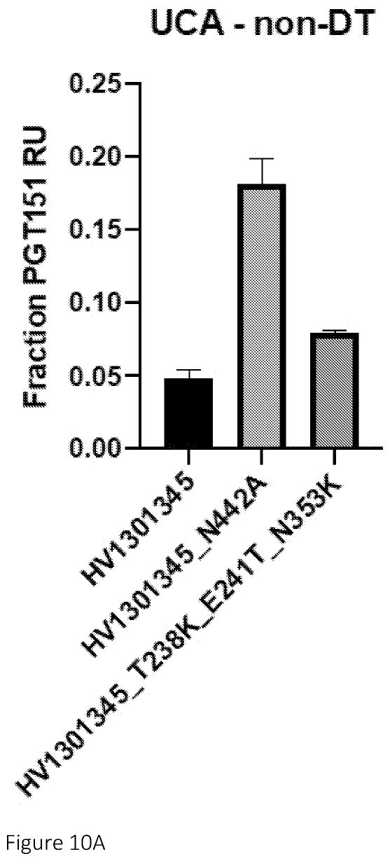

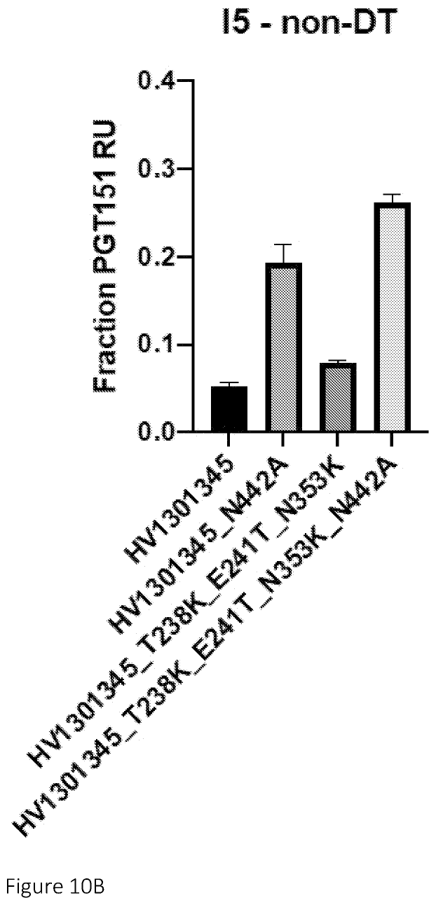

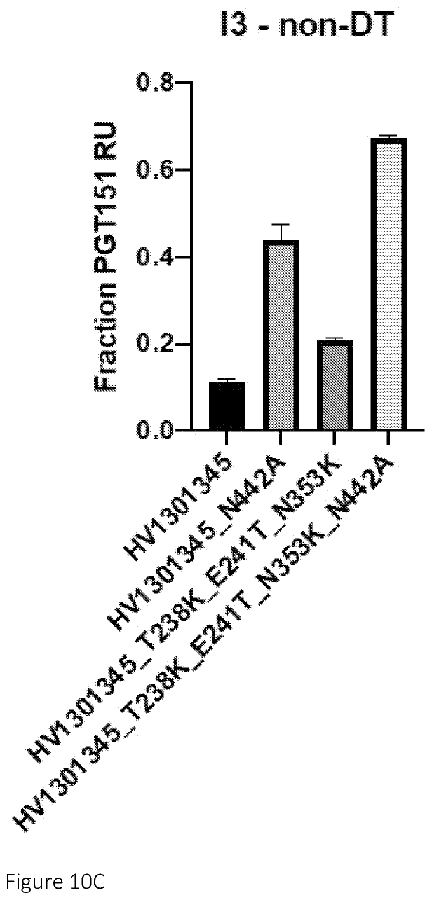

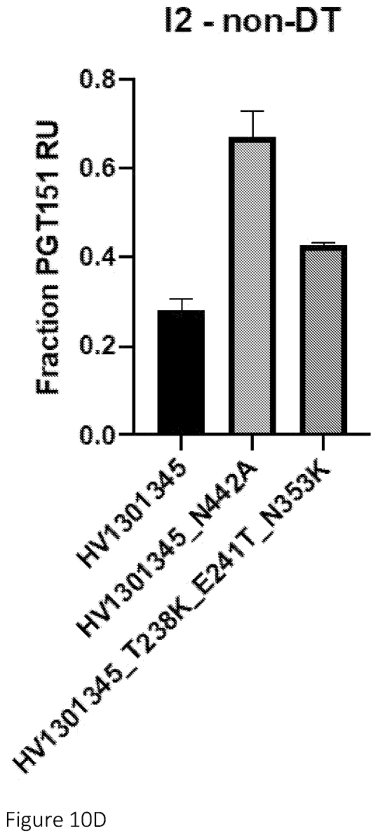

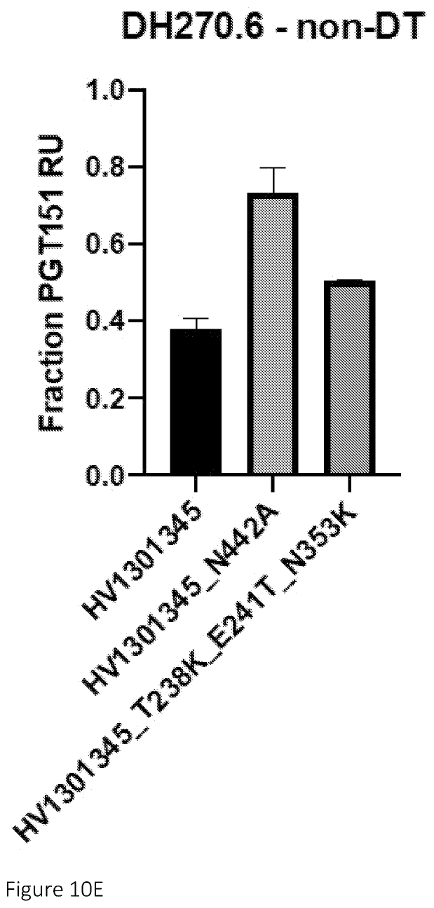

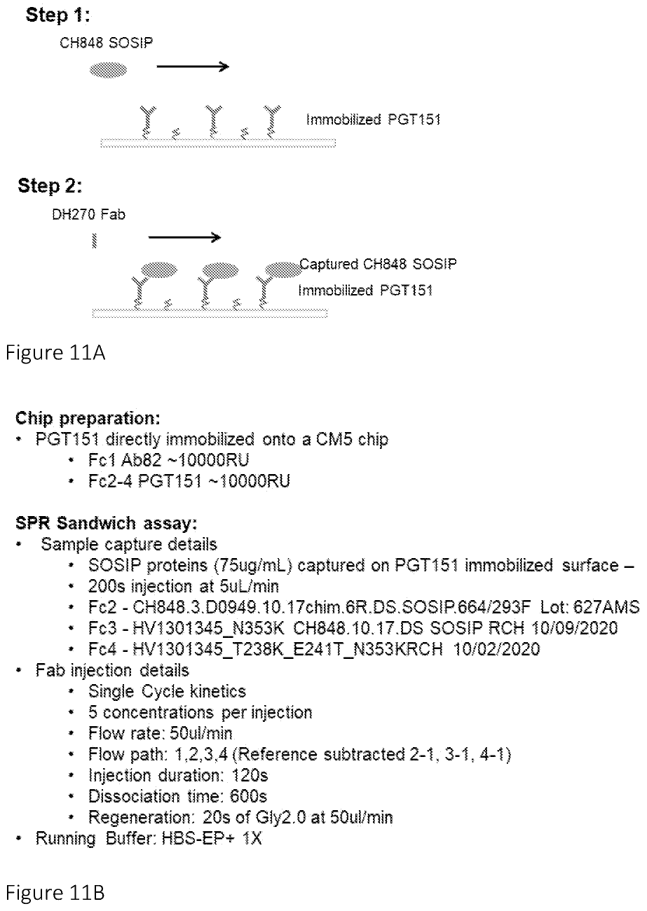

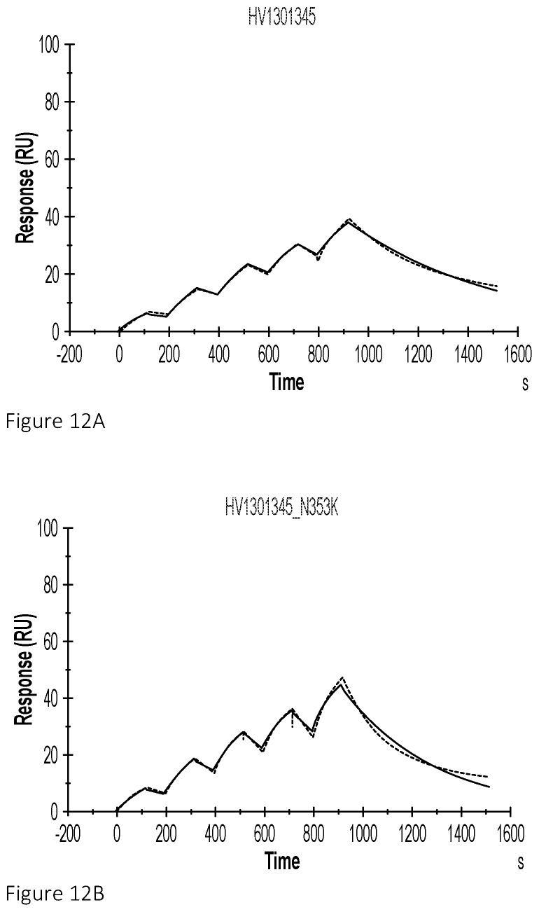

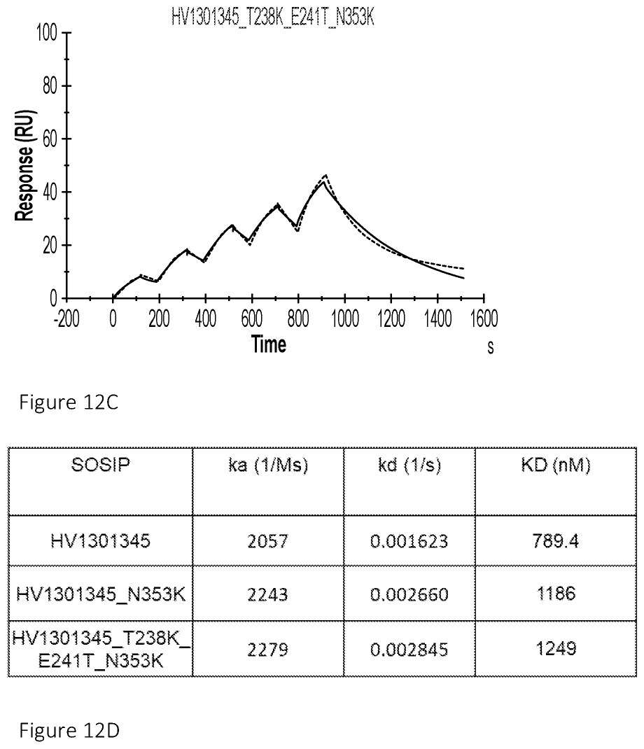

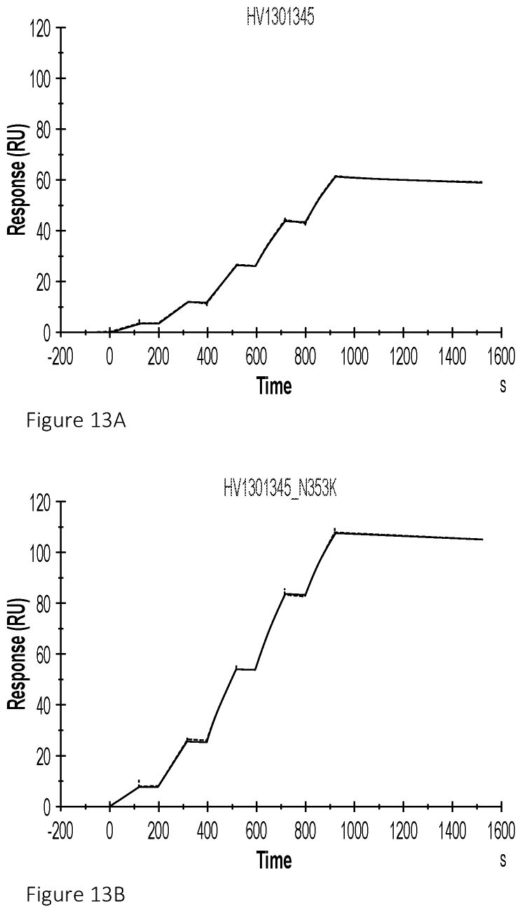

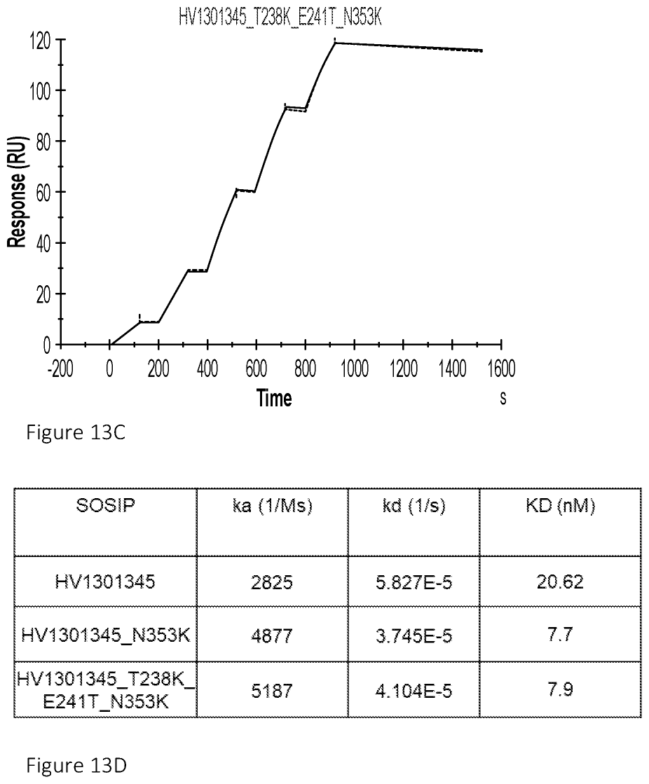

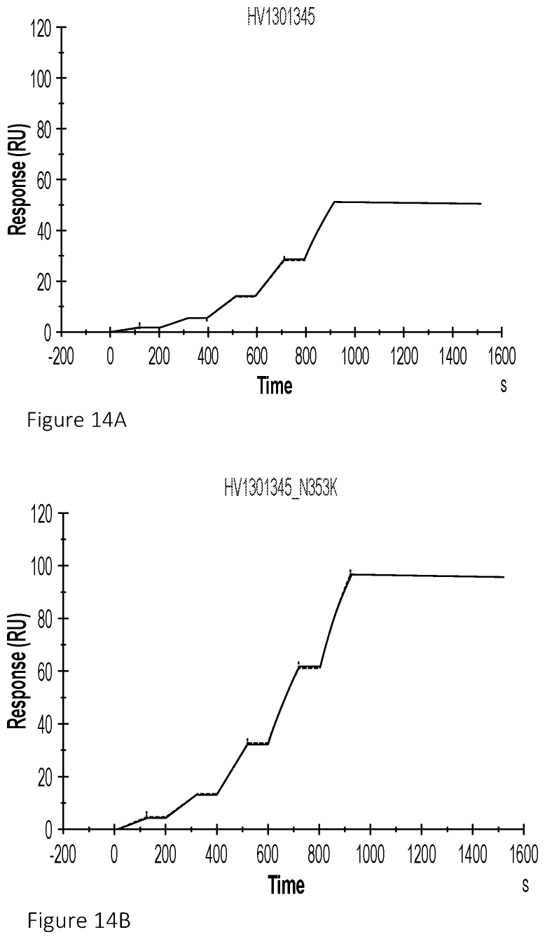

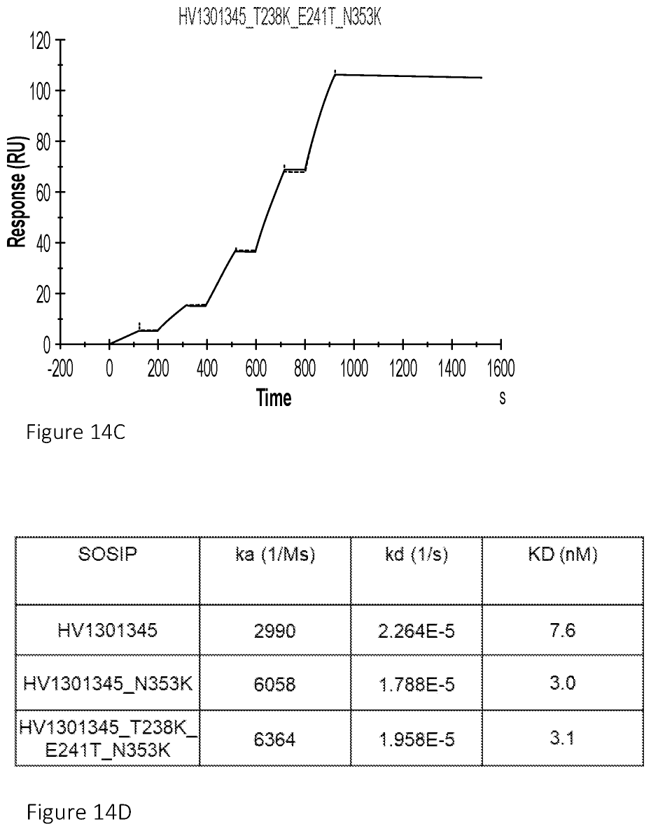

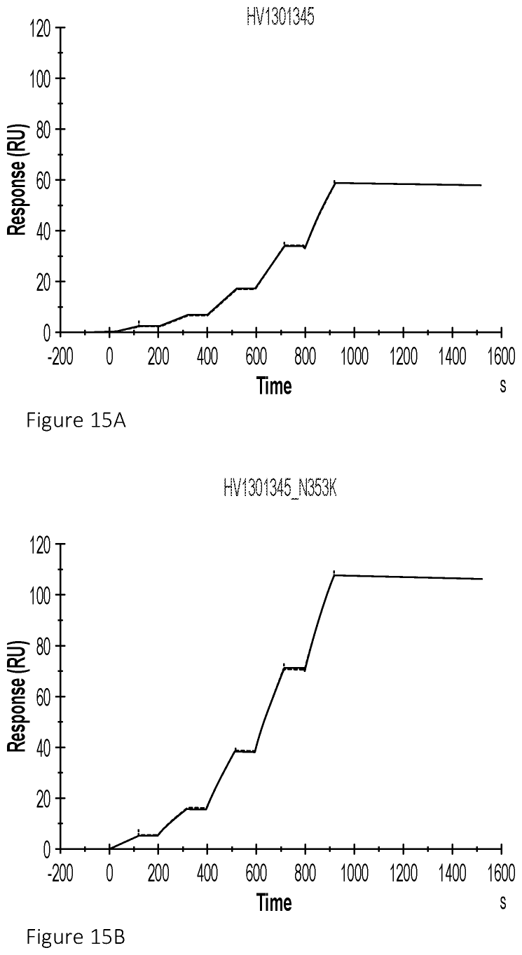

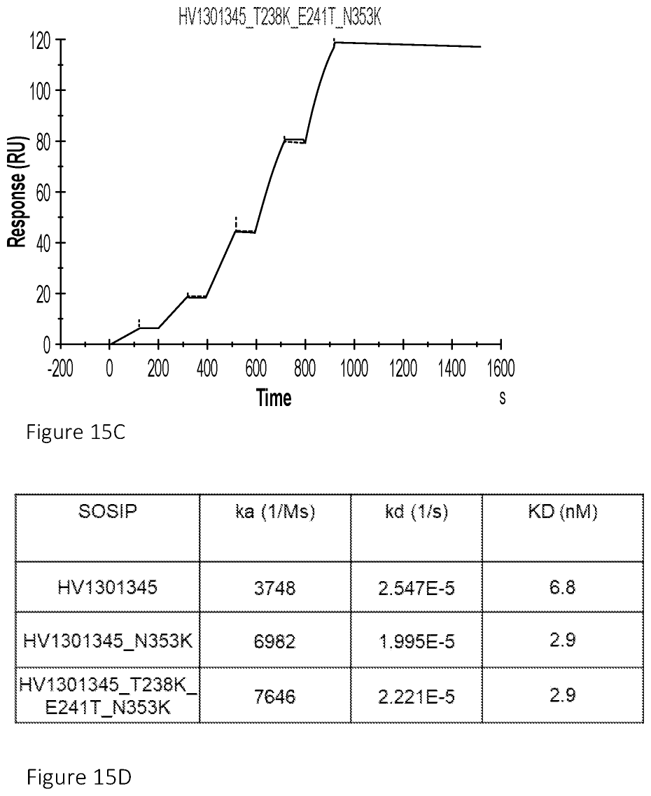

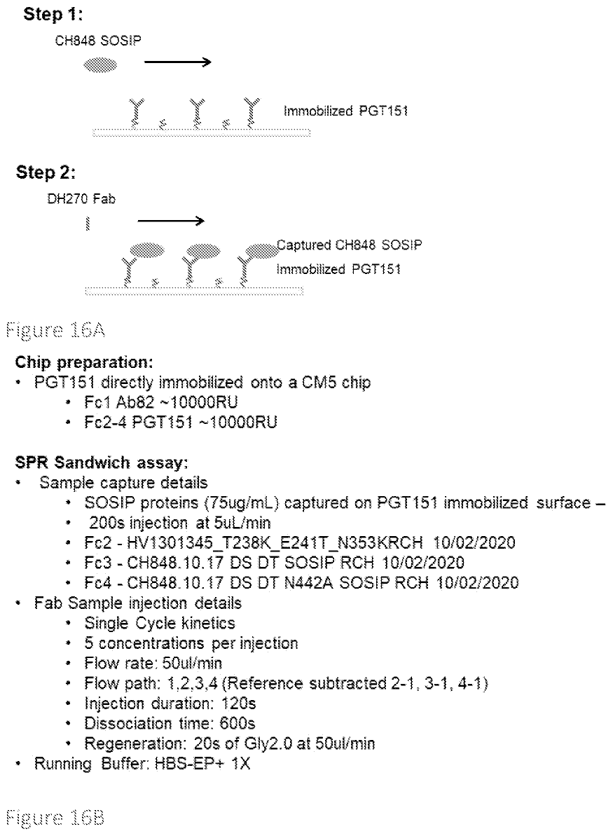

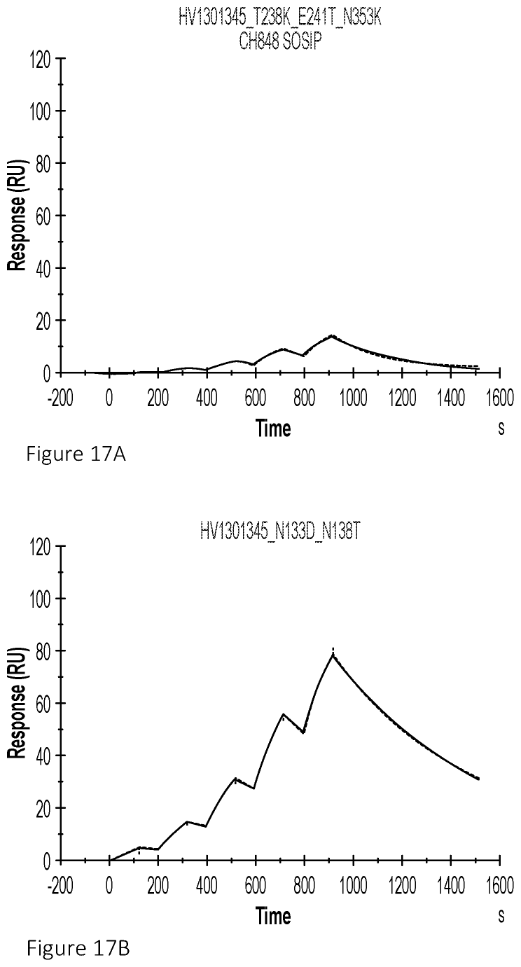

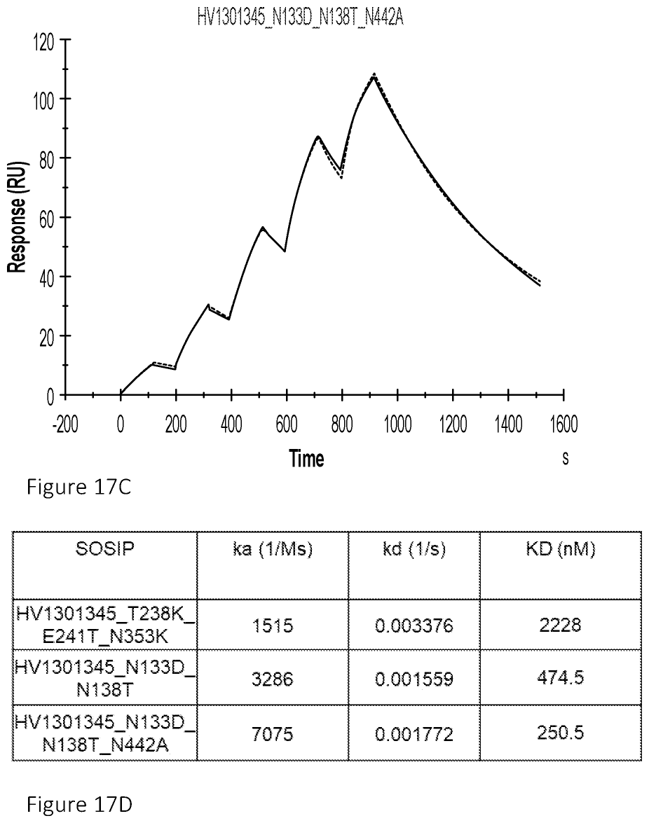

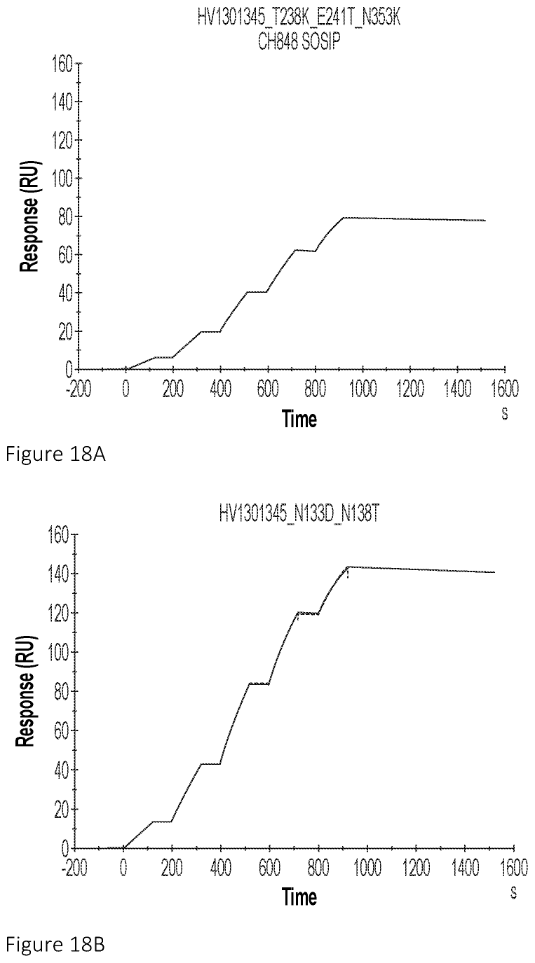

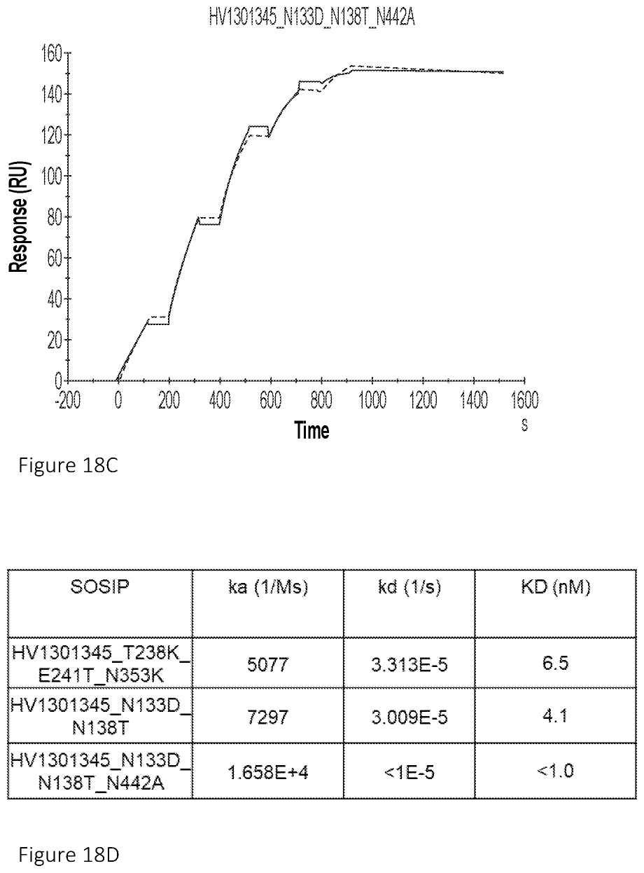

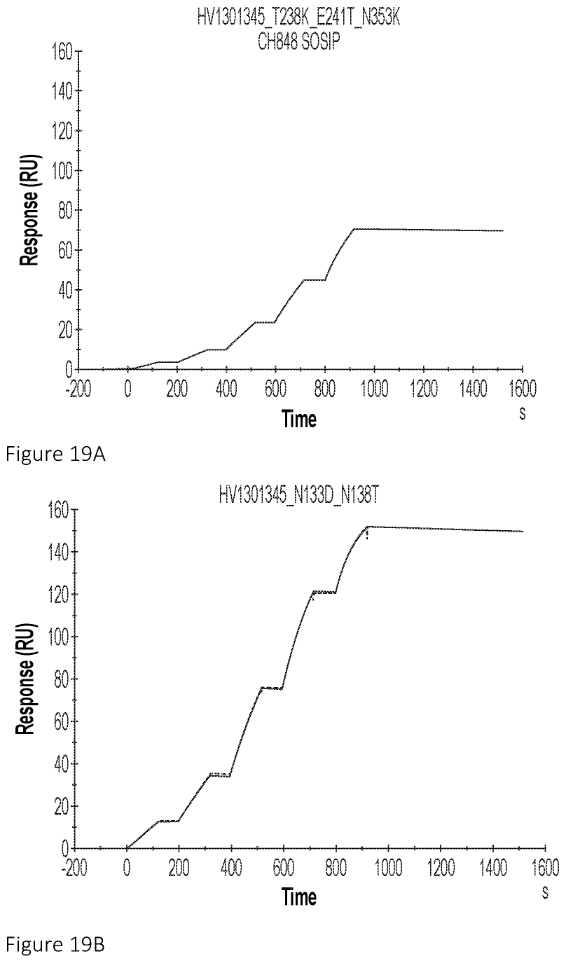

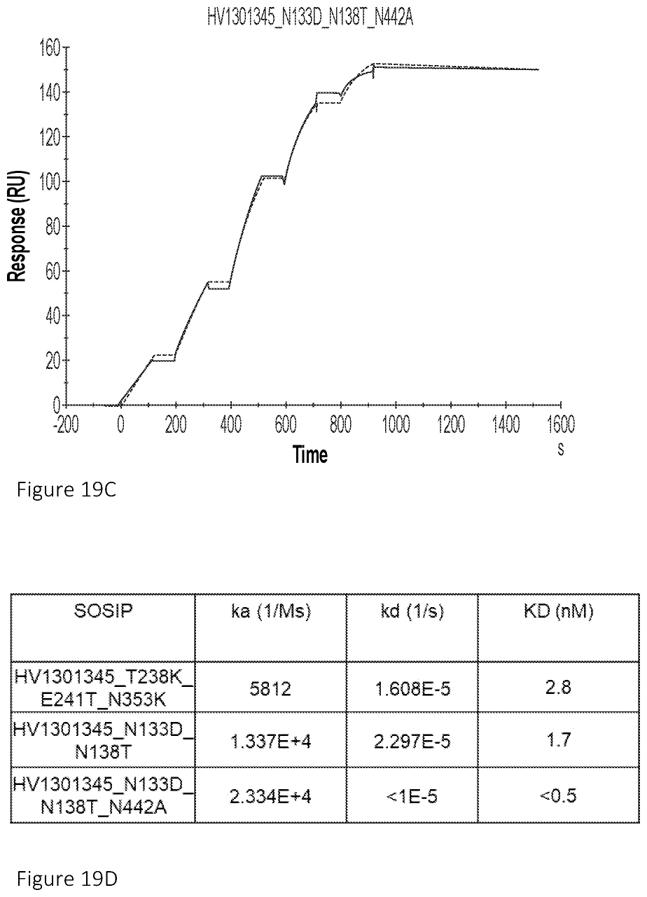

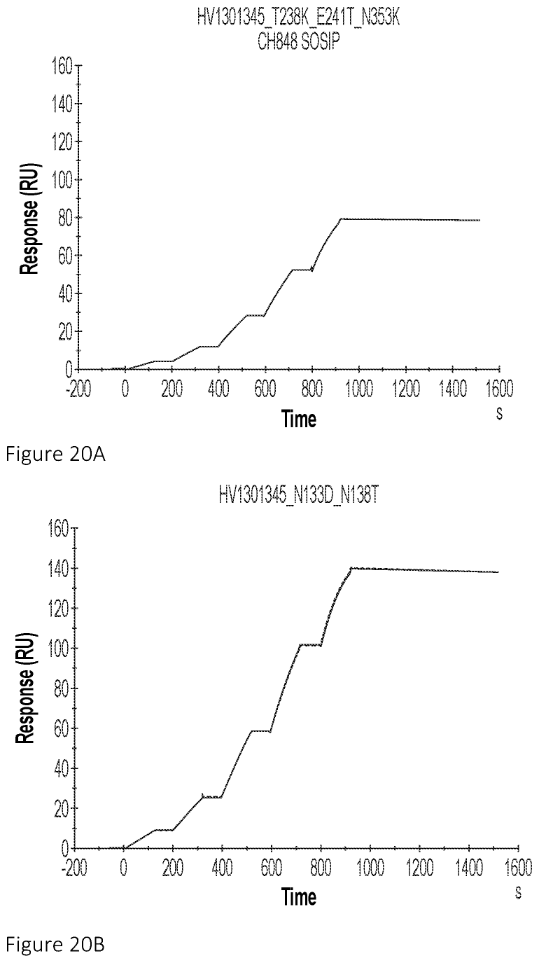

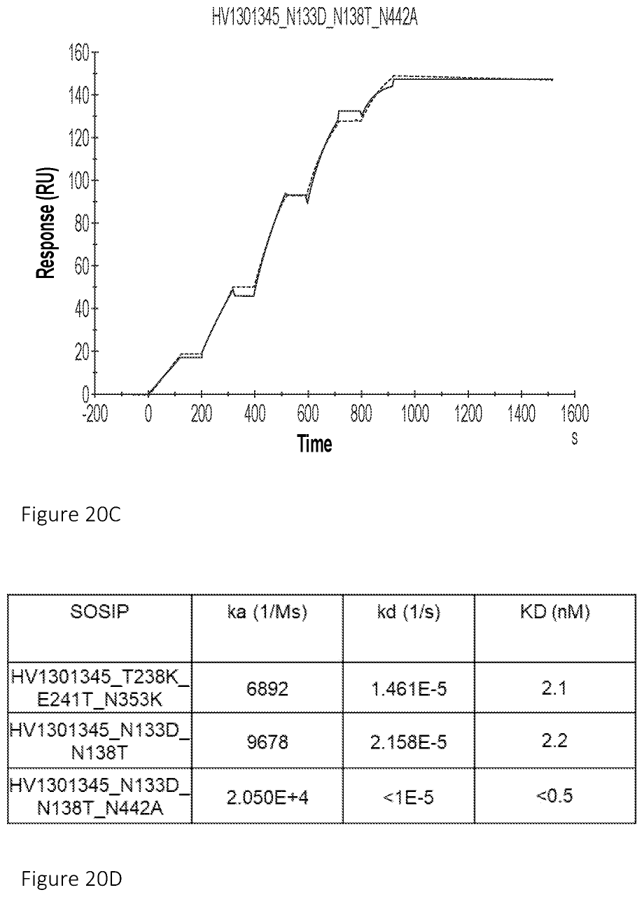

Another approach to multimerize expression constructs uses staphylococcus sortase A transpeptidase ligation to conjugate inventive envelope trimers to cholesterol. The trimers can then be embedded into liposomes via the conjugated cholesterol. To conjugate the trimer to cholesterol, a C-terminal LPXTG tag, where X signifies any amino acid, such as Ala, Ser, Glu, or a N-terminal pentaglycine repeat tag is added to the envelope trimer gene. Cholesterol is also synthesized with these two tags. Sortase A is then used to covalently bond the tagged envelope to the cholesterol. The sortase A-tagged trimer protein can also be used to conjugate the trimer to other peptides, proteins, or fluorescent labels. In non-limiting embodiments, the sortase A tagged trimers are conjugated to ferritin to form nanoparticles. See FIG. 8.

The invention provides design of envelopes and trimer designs wherein the envelope comprises a linker which permits addition of a lipid, such as but not limited to cholesterol, via a sortase A reaction. See e.g. Tsukiji, S. and Nagamune, T. (2009), Sortase-Mediated Ligation: A Gift from Gram-Positive Bacteria to Protein Engineering. ChemBioChem, 10: 787-798. doi: 10.1002/cbic.200800724; Proft, T. Sortase-mediated protein ligation: an emerging biotechnology tool for protein modification and immobilisation. Biotechnol Lett (2010) 32: 1. doi: 10.1007/s10529-009-0116-0; Lena Schmohl, Dirk Schwarzer, Sortase-mediated ligations for the site-specific modification of proteins, Current Opinion in Chemical Biology, Volume 22, October 2014, Pages 122-128, ISSN 1367-5931, dx.doi.org/10.1016/j.cbpa.2014.09.020; Tabata et al. Anticancer Res. 2015 August; 35(8):4411-7; Pritz et al. J. Org. Chem. 2007, 72, 3909-3912.

The lipid modified envelopes and trimers can be formulated as liposomes. Any suitable liposome composition is encompassed.

Non-limiting embodiments of envelope designs for use in sortase A reaction are shown in FIG. 24 B-D of WO2017/151801, incorporated by reference in its entirety.

Additional sortase linkers can be used so long as their position allows multimerization of the envelopes. In a non-limiting embodiment, a C-terminal tag is LPXTG (SEQ ID NO: [ ]), where X signifies any amino acid but Ala, Ser, Glu, or a N-terminal pentaglycine repeat tag is added to the envelope trimer gene. In a non-limiting embodiment, a C-terminal tag is LPXTGG (SEQ ID NO: [ ]), where X signifies any amino acid, such as Ala, Ser, Glu.

For development as a vaccine immunogen, we have also created multimeric nanoparticles that comprise and/or display HIV envelope protein or fragments on their surface.

The nanoparticle immunogens are composed of various forms of HIV-envelope protein, e.g. without limitation envelope trimer, and self-assembling protein, e.g. without limitation ferritin protein. Any suitable ferritin can be used in the immunogens of the invention. In non-limiting embodiments, the ferritin is derived from Helicobacter pylori. In non-limiting embodiments, the ferritin is insect ferritin. In non-limiting embodiments, each nanoparticle displays 24 copies of the spike protein on its surface.

Multimeric complexes presenting of antigens—nanoparticles. Presenting multiple copies of antigens to B cells has been a longstanding approach to improving B cell receptor recognition and antigen uptake (See Batista et al. EMBO J. 2000 Feb. 15; 19(4): 513-520). The improved recognition of antigen is due to the avid interaction of multiple antigens with multiple B cell receptors on a single B cells, which results in clustering of B cells and stronger cell signaling. Furthermore, multimeric presentation improves antigen binding to mannose binding lectin which promotes antigen trafficking to B cell follicles. Self-assembling complexes comprising multiple copies of an antigen are one strategy of immunogen design approach for arraying multiple copies of an antigen for recognition by the B cell receptors on B cells (Kanekiyo, M., Wei, C. J., Yassine, H. M., McTamney, P. M., Boyington, J. C., Whittle, J. R., Rao, S. S., Kong, W. P., Wang, L., and Nabel, G. J. (2013). Self-assembling influenza nanoparticle vaccines elicit broadly neutralizing H1N1 antibodies. Nature 499, 102-106; Ueda, G., Antanasijevic, A., Fallas, J. A., Sheffler, W., Copps, J., Ellis, D., Hutchinson, G. B., Moyer, A., Yasmeen, A., Tsybovsky, Y., et al. (2020). Tailored design of protein nanoparticle scaffolds for multivalent presentation of viral glycoprotein antigens. Elife).

In some embodiments, the gene of an antigen can be fused via a linker/spacer/tag to a gene of a protein which can self-assemble. Upon translation, a fusion protein is made that can self-assemble into a multimeric complex—also referred to as a nanoparticle displaying multiple copies of the antigen. In some embodiments, the protein antigen can be conjugated to the self-assembling protein via an enzymatic reaction, thereby forming a nanoparticle displaying multiple copies of the antigen. Non-limiting embodiments of enzymatic conjugation include without limitation sortase mediated conjugation. In some embodiments, linkers for use in any of the designs of the invention can be 2-50 amino acids long, e.g. 2, 3, 4, 5, 6, 7, 8, 9, 10, 11, 12, 13, 14, 15, 16, 17, 18, 19, 20, 25, 30, 35, 40, 45, or 50 amino acids long. In certain embodiments, these linkers comprise glycine and serine amino acid in any suitable combination, and/or repeating units of combinations of glycine, serine and/or alanine.

Ferritin is a well-known protein that self-assembles into a hollow particle composed of repeating subunits. In some species ferritin nanoparticles are composed of 24 copies of a single subunit, whereas in other species it is composed of 12 copies each of two subunits.

Non-limiting embodiments of sortase linkers/tags can be used so long as their position allows multimerization of the envelopes. In a non-limiting embodiment, a C-terminal tag is LPXTG (SEQ ID NO: [ ]), where X signifies any amino acid but Ala, Ser, Glu, or a N-terminal pentaglycine repeat tag is added to the envelope trimer gene. In a non-limiting embodiment, a C-terminal tag is LPXTGG (SEQ ID NO: [ ]), where X signifies any amino acid, such as Ala, Ser, Glu.

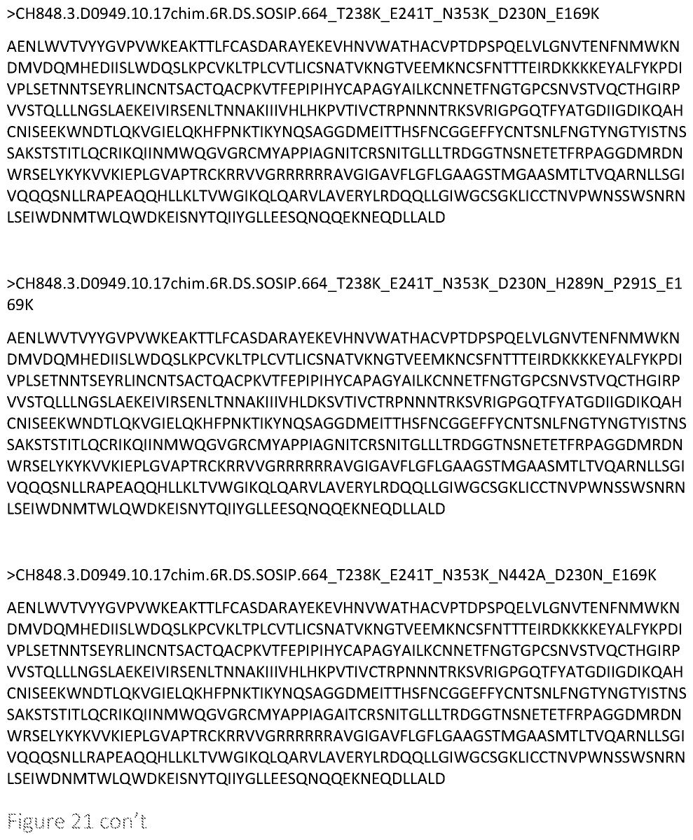

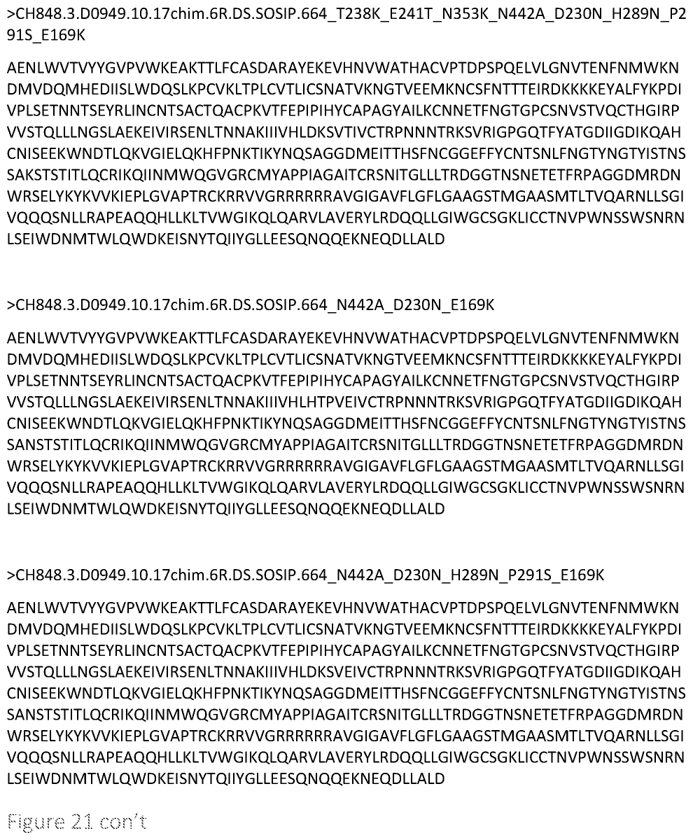

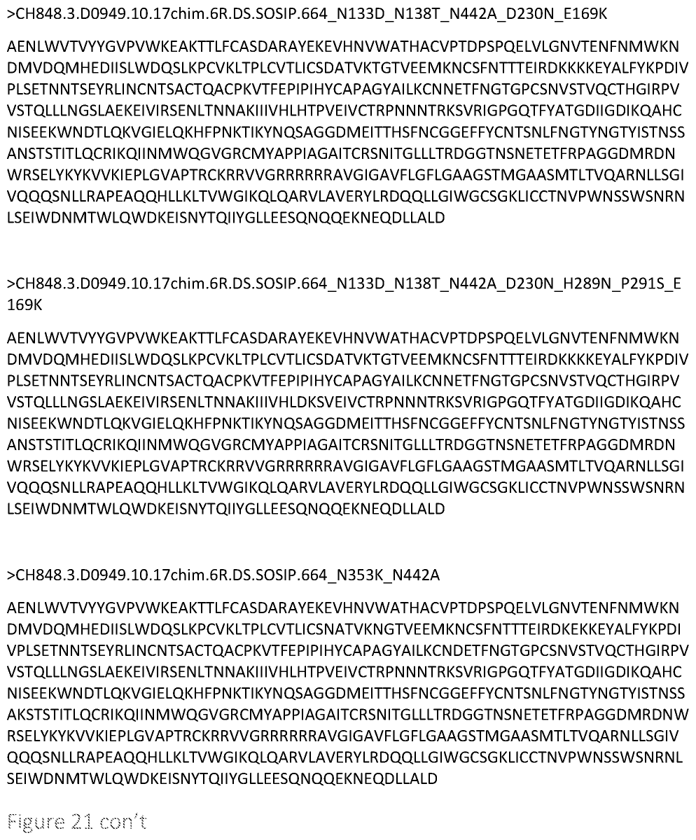

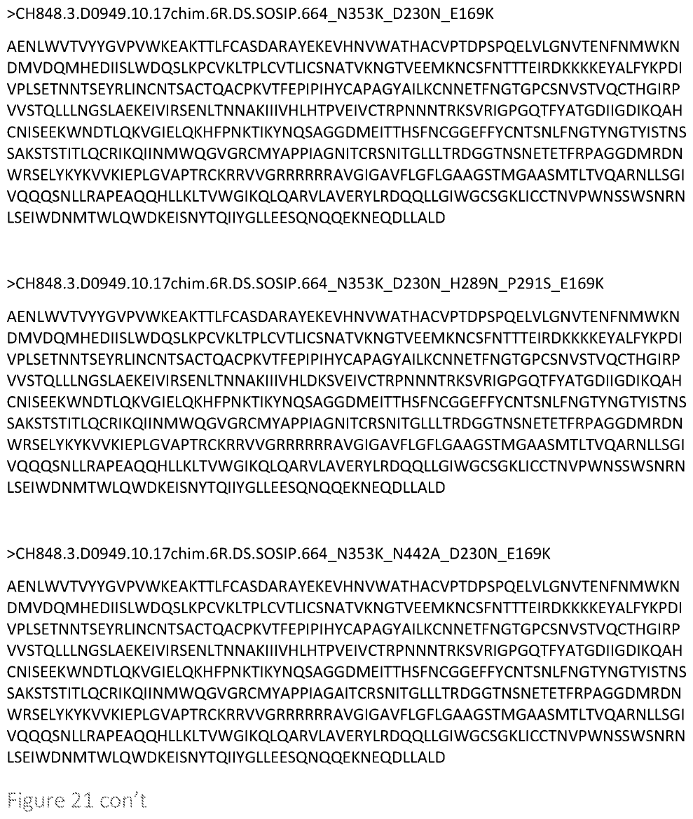

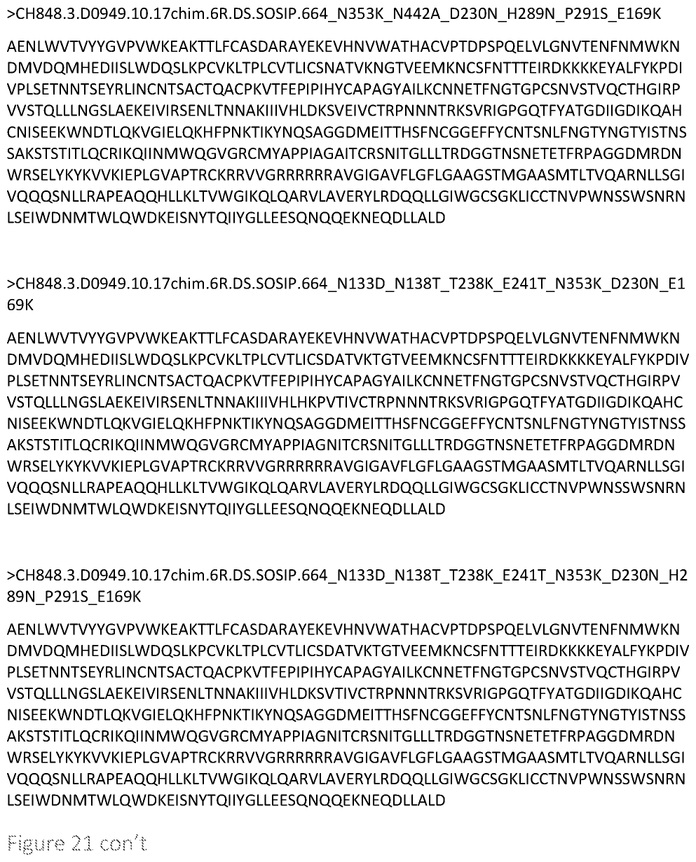

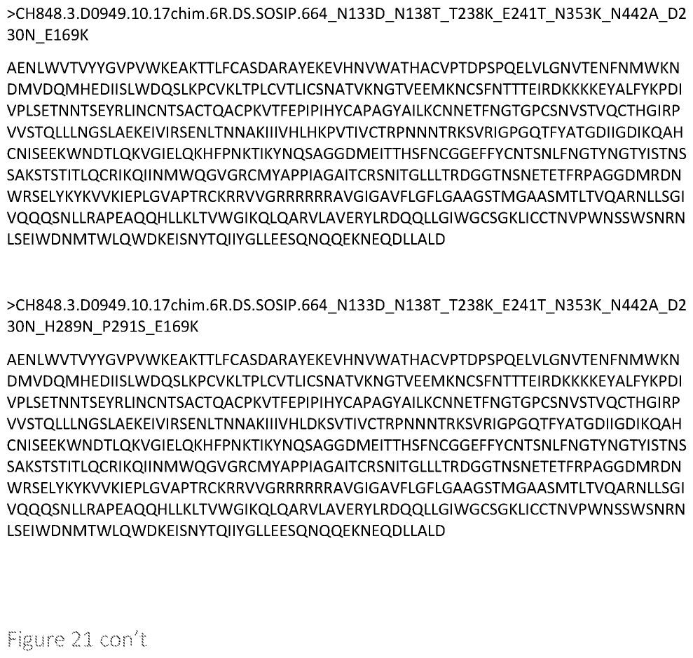

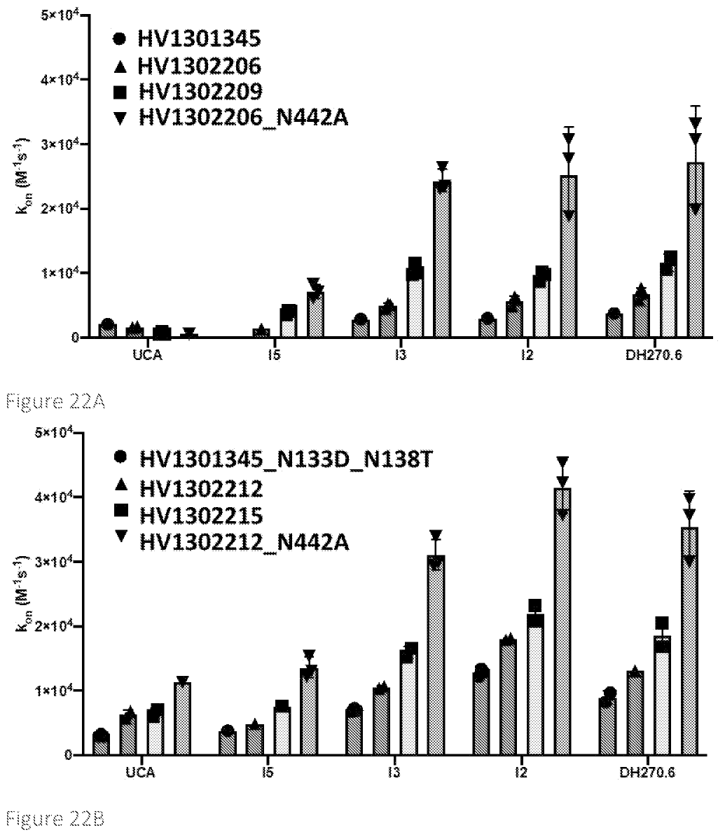

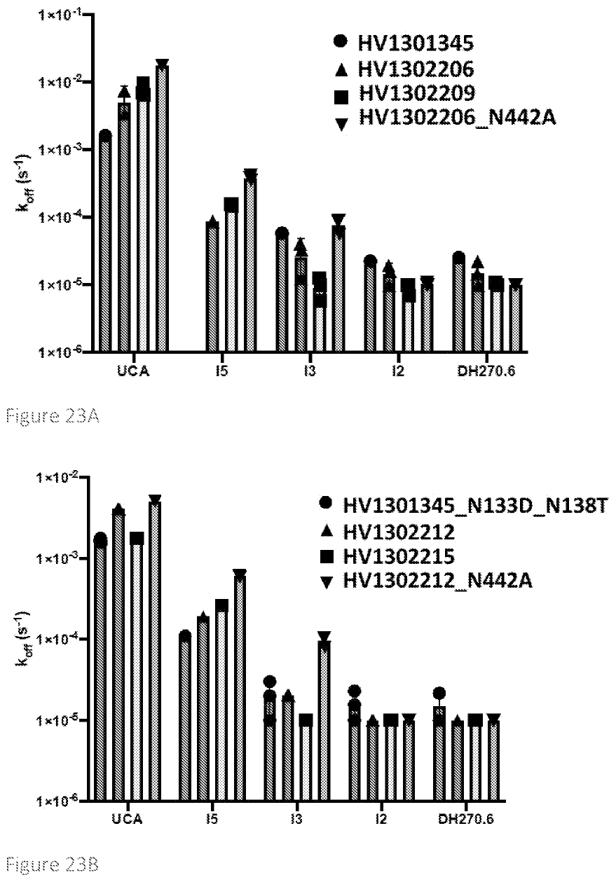

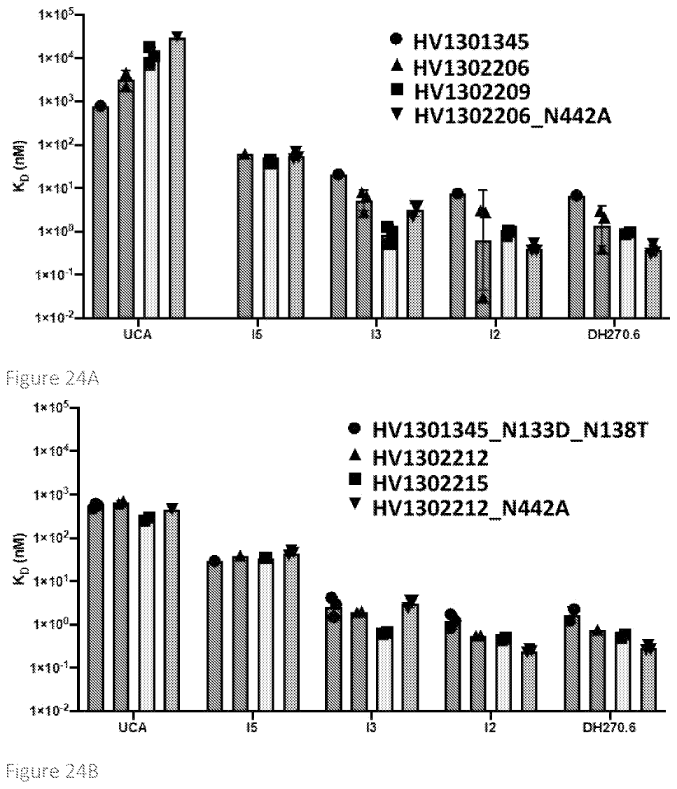

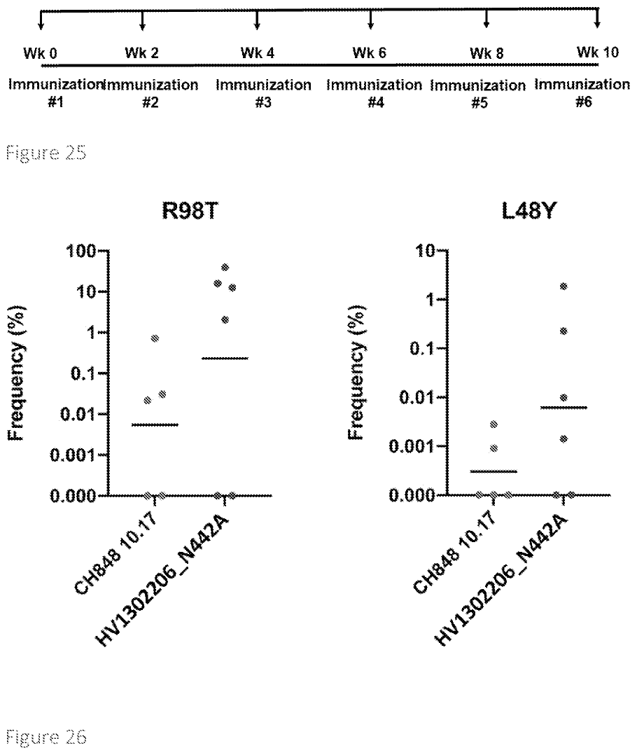

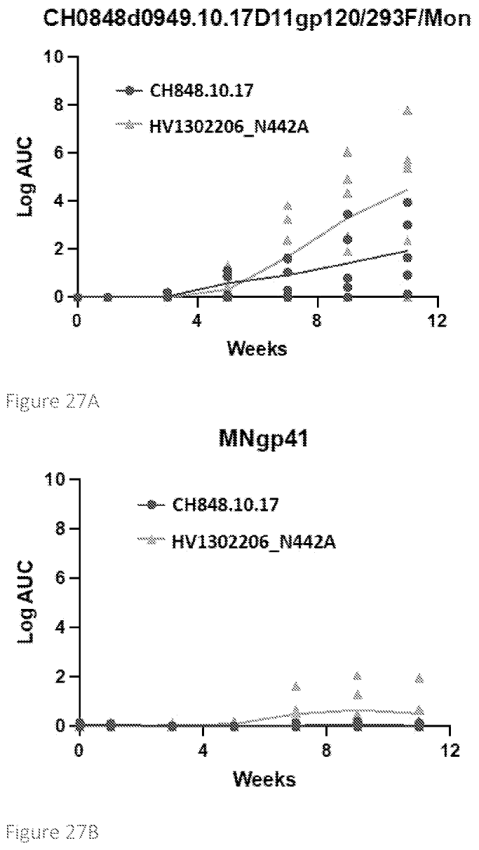

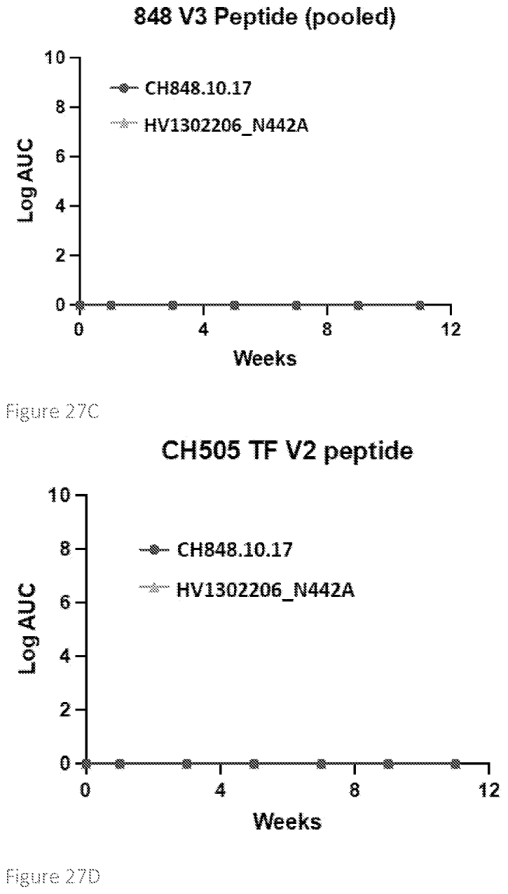

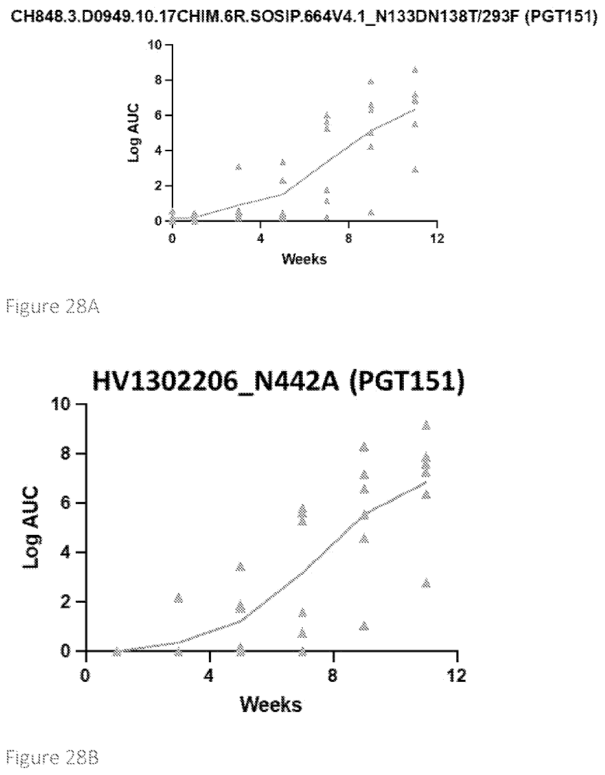

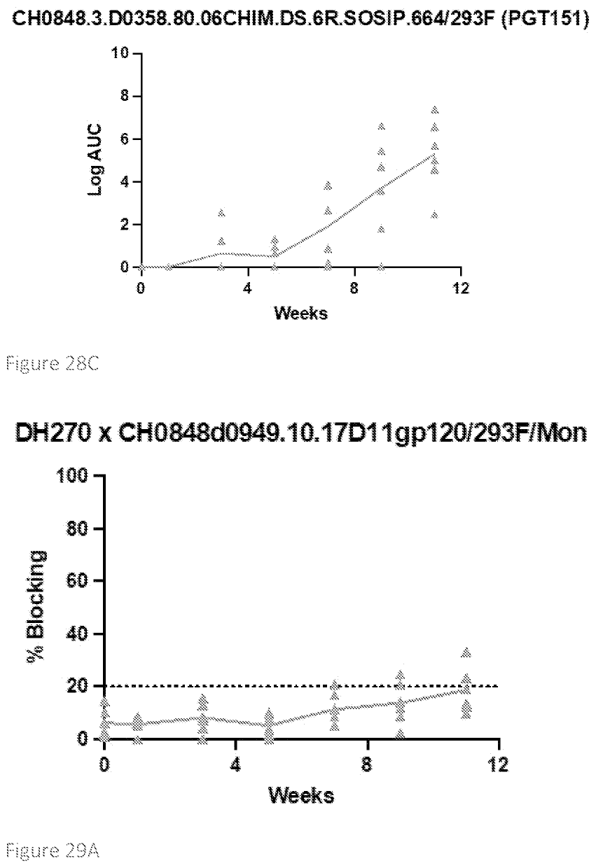

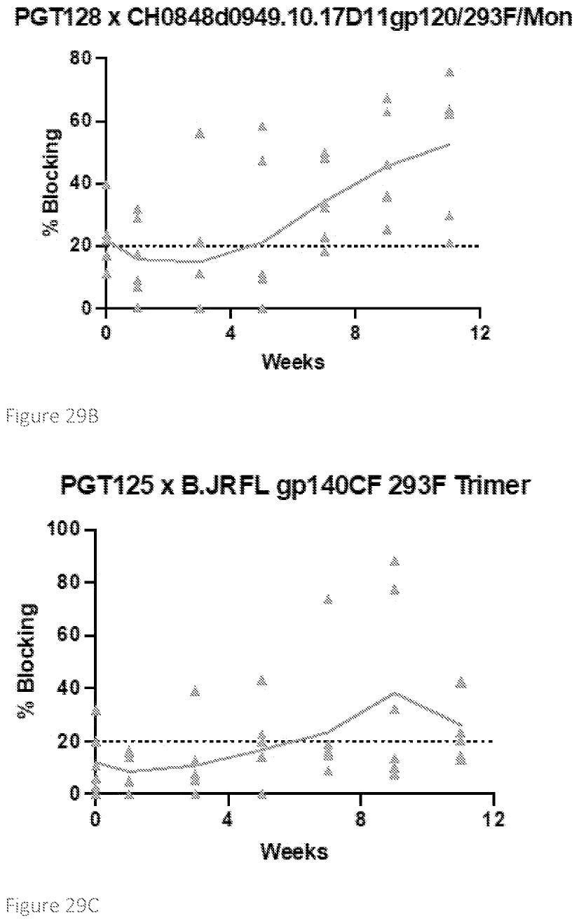

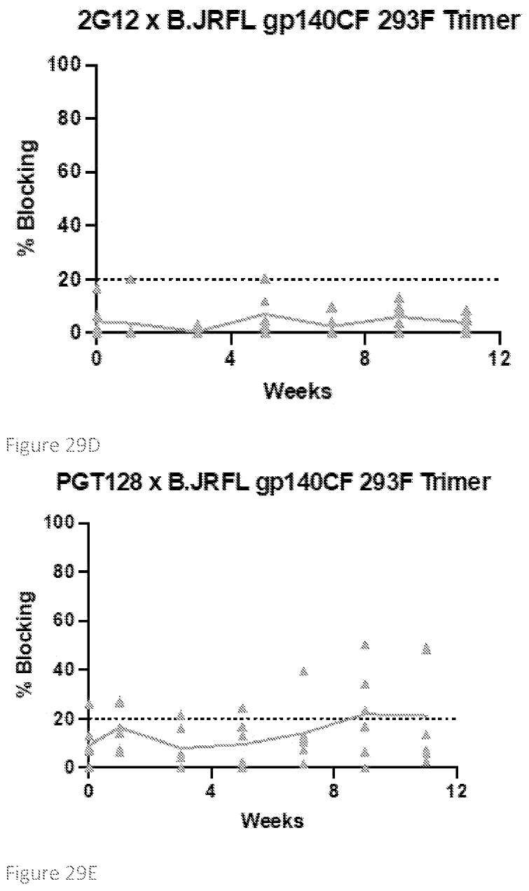

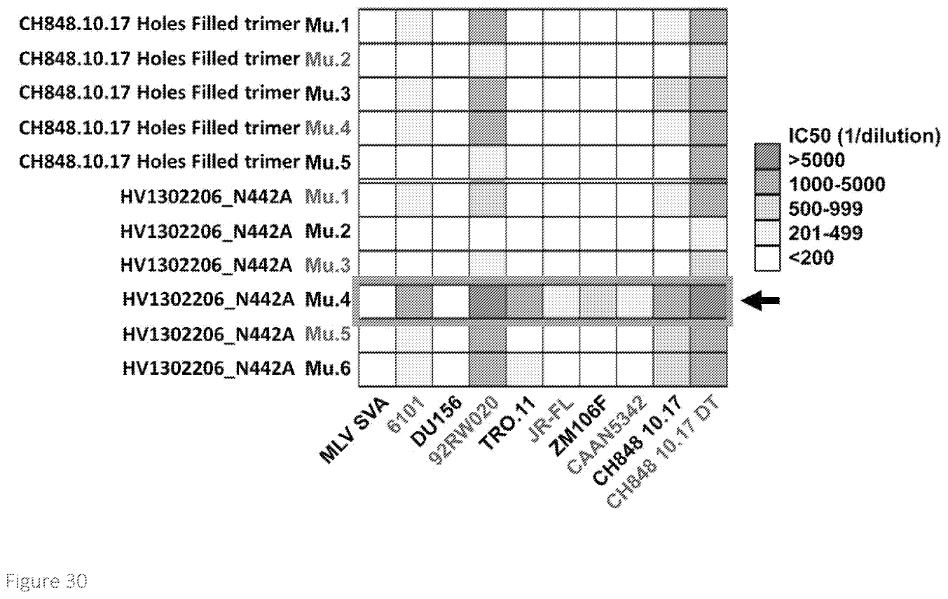

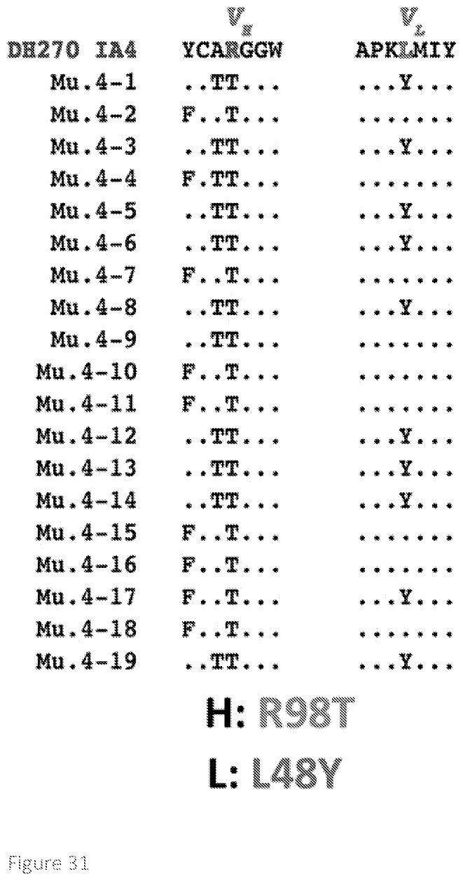

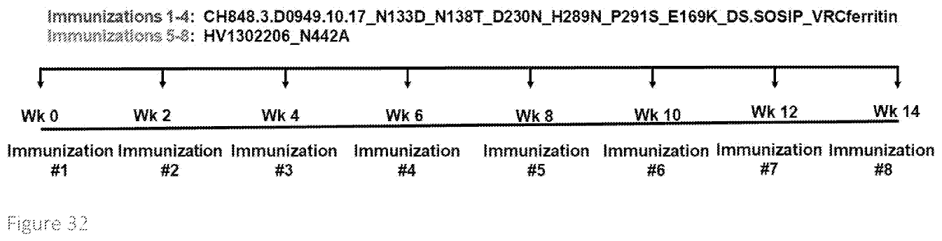

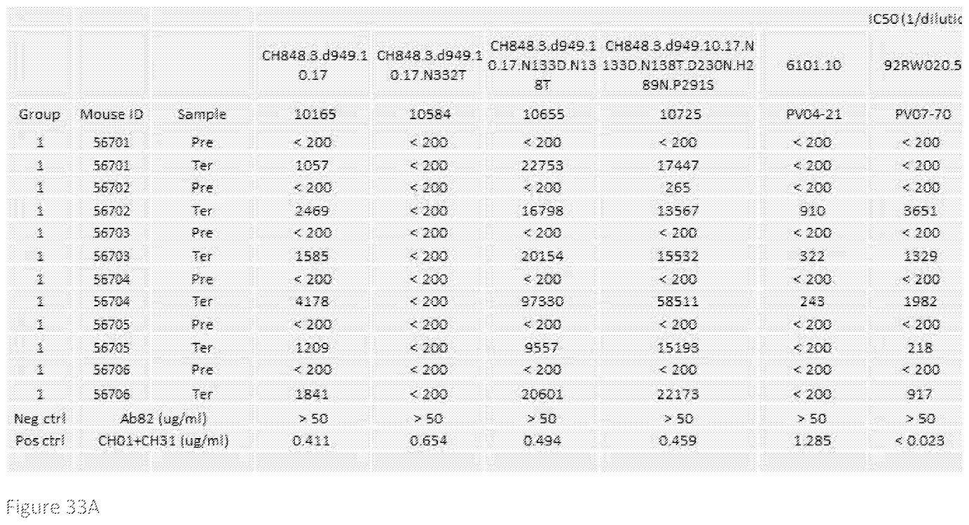

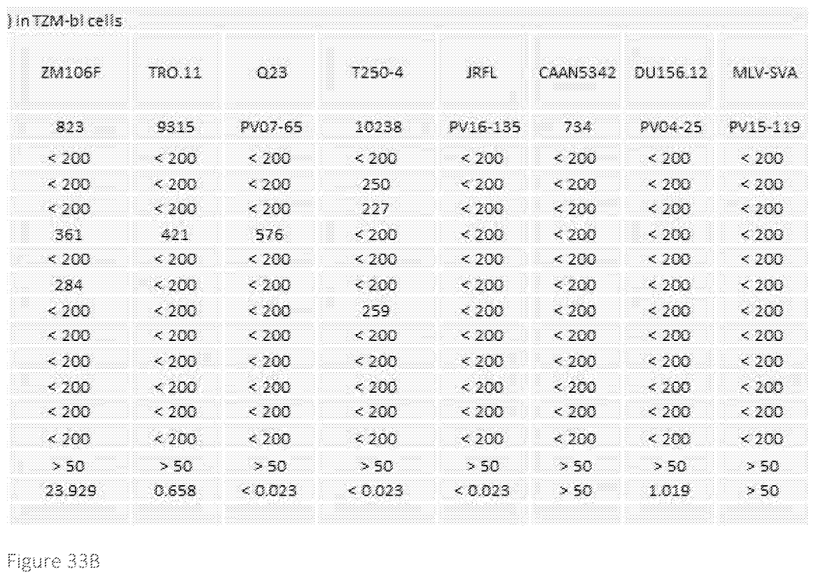

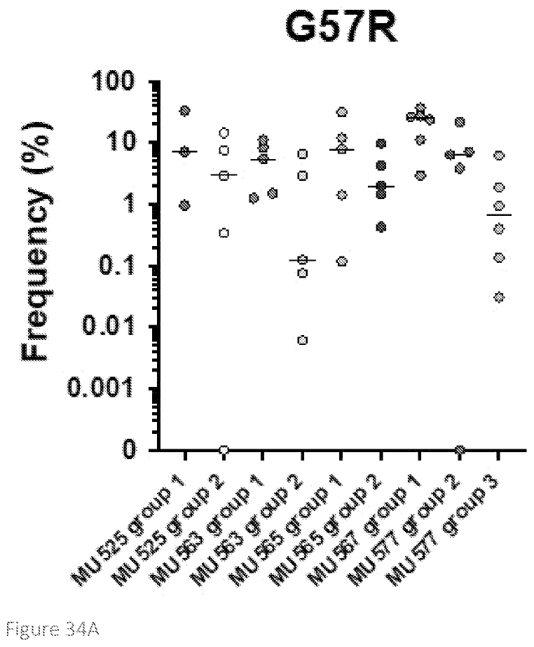

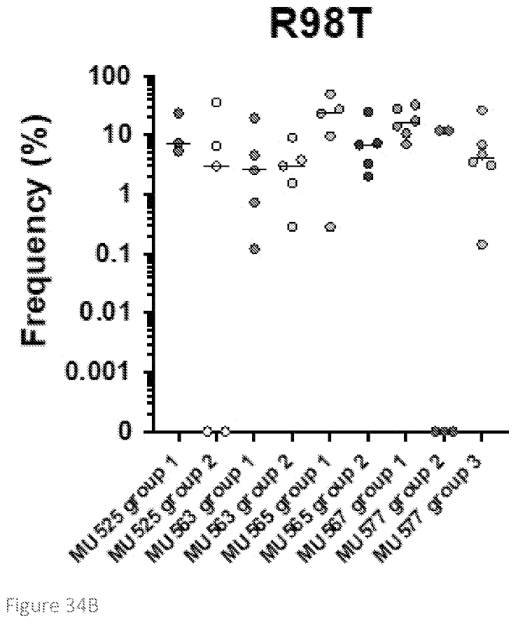

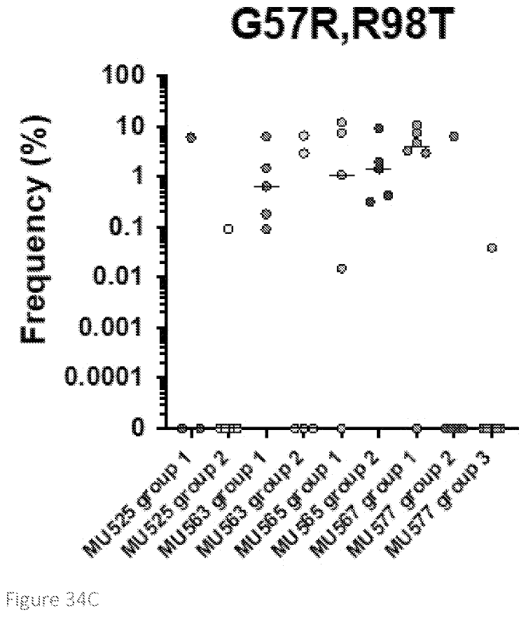

Table 1A and 1B show a summary of non-limiting embodiments of sequences if the invention. Non-limiting embodiments of amino acid sequences of the invention comprising encounter amino acid changes are listed in FIG. 7 and FIG. 21. The sequences in FIG. 7 and FIG. 21 are SOSIP protomers. A skilled artisan can readily incorporate the encounter amino acid changes in any other suitable envelope or envelope form.

The invention provides any other forms, e.g. without limitation trimers or nanoparticles, of the sequences described herein. For non-limiting embodiments of additional stabilized trimers see WO2014/042669 (DU4061), WO/2017151801 (DU4716), WO/2017152146 (DU4918) and WO/2018161049 (DU4918), PCT/US2019/049431 (DU6550), and PCT/US2019/049662 (DU6546) which are incorporated by reference in their entirety.

Throughout the application envelope HV1301345_N133D_N138T is also referred as DT. Throughout the application envelope HV1301345_is also referred as non-DT.

TABLE 1A
Summary of envelope amino acid positions involved in antibody encounter complexes and sequence names. The second column identifies encounter mutation amino acid position
based on the first amino acid in the sequence in FIG. 7. The third column identifies encounter amino acid position based on HXB2 numbering. Provided are non-limiting
examples of changes to specific amino acid at selected position. A skilled artisan can readily determine additional changes, based on well-known properties and considerations
of nature and properties of amino acids, at these selected positions. Non-limiting embodiments of amino acid sequences are shown in FIG. 7.

| | | | PARENT | |
|---|---|---|---|---|
| | CONSTRUCT | HXB2 Numbers | SEQUENCE | FULL ID |
| | Encounter Tryptophan Mutations | | | |
| | HV1301345 | HV1301345 | HV1301345 | CH848.3.D0949.10.17chim.6R.DS.SOSIP.664 |
| | HV1301345_V243W | HV1301345_V295W | HV1301345 | CH848.3.D0949.10.17chim.6R.DS.SOSIP.664_V243W |
| | HV1301345_T245W | HV1301345_T297W | HV1301345 | CH848.3.D0949.10.17chim.6R.DS.SOSIP.664_T245W |
| | HV1301345_T388W | HV1301345_T444W | HV1301345 | CH848.3.D0949.10.17chim.6R.DS.SOSIP.664_T388W |
| | HV1301345_H277W | HV1301345_H330W | HV1301345 | CH848.3.D0949.10.17chim.6R.DS.SOSIP.664_H277W |
| | HV1301345_T359W | HV1301345_T415W | HV1301345 | CH848.3.D0949.10.17chim.6R.DS.SOSIP.664_T359W |
| | HV1301345_T357W | HV1301345_T413W | HV1301345 | CH848.3.D0949.10.17chim.6R.DS.SOSIP.664_T357W |
| | HV1301345_S281W | HV1301345_S334W | HV1301345 | CH848.3.D0949.10.17chim.6R.DS.SOSIP.664_S281W |
| | HV1301345_E241W | HV1301345_E293W | HV1301345 | CH848.3.D0949.10.17chim.6R.DS.SOSIP.664_E241W |
| | HV1301345_R390W | HV1301345_R446W | HV1301345 | CH848.3.D0949.10.17chim.6R.DS.SOSIP.664_R390W |
| | Encounter Designs with V1 N-glycosylation Sites | | | |
| | HV1301345_A352N_N353K | HV1301345_A407N_N408K | HV1301345 | CH848.3.D0949.10.17chim.6R.DS.SOSIP.664_A352N_N353K |
| | HV1301345_E241T_N353K | HV1301345_E293T_N408K | HV1301345 | CH848.3.D0949.10.17chim.6R.DS.SOSIP.664_E241T_N353K |
| | HV1301345_T238K_E241T_A352N_{—} | HV1301345_T290K_E293T_A407N_{—} | HV1301345 | CH848.3.D0949.10.17chim.6R.DS.SOSIP.664_T238K_E241T_A352N_{—} |
| | N353K | N408K | | N353K |
| | HV1301345_E241T | HV1301345_E293T | HV1301345 | CH848.3.D0949.10.17chim.6R.DS.SOSIP.664_E241T |
| | HV1301345_E241T_S281K | HV1301345_E293T_S334K | HV1301345 | CH848.3.D0949.10.17chim.6R.DS.SOSIP.664_E24IT_S281K |
| | HV1301345_E241T_T355K | HV1301345_E293T_T411K | HV1301345 | CH848.3.D0949.10.17chim.6R.DS.SOSIP.664_E241T_T355K |
| | HV1301345_E241T_T238K_S281K | HV1301345_E293T_T290K_S334K | HV1301345 | CH848.3.D0949.10.17chim.6R.DS.SOSIP.664_E241T_T238K_S281K |
| | HV1301345_E241T_V243T | HV1301345_E293T_V295T | HV1301345 | CH848.3.D0949.10.17chim.6R.DS.SOSIP.664_E241T_V243T |
| | HV1301345_T238K | HV1301345_T290K | HV1301345 | CH848.3.D0949.10.17chim.6R.DS.SOSIP.664_T238K |
| | HV1301345_V243T | HV1301345_V295T | HV1301345 | CH848.3.D0949.10.17chim.6R.DS.SOSIP.664_V243T |
| | HV1301345_V243T_E241K | HV1301345_V295T_E293K | HV1301345 | CH848.3.D0949.10.17chim.6R.DS.SOSIP.664_V243T_E241K |
| | HV1301345_V243T_E241K_S354K | HV1301345_V295T_E293K_S409K | HV1301345 | CH848.3.D0949.10.17chim.6R.DS.SOSIP.664_V243T_E241K_S354K |
| | HV1301345_N248C_I273C | HV1301345_N300C_I327C | HV1301345 | CH848.3.D0949.10.17chim.6R.DS.SOSIP.664_N248C_I273C |
| | HV1301345_N250C_I269C | HV1301345_N302C_I322C | HV1301345 | CH848.3.D0949.10.17chim.6R.DS.SOSIP.664_N250C_I269C |
| | HV1301345_N250C_T266C | HV1301345_N302C_T320C | HV1301345 | CH848.3.D0949.10.17chim.6R.DS.SOSIP.664_N250C_T266C |
| | HV1301345_L121C_T266C | HV1301345_L173C_T320C | HV1301345 | CH848.3.D0949.10.17chim.6R.DS.SOSIP.664_L121C_T266C |
| | HV1301345_R246C_A276C | HV1301345_R298C_A329C | HV1301345 | CH848.3.D0949.10.17chim.6R.DS.SOSIP.664_R246C_A276C |
| | HV1301345_R246C_A276C_E327L | HV1301345_R298C_A329C_E381L | HV1301345 | CH848.3.D0949.10.17chim.6R.DS.SOSIP.664_R246C_A276C_E327L |
| | HV1301345_I270N | HV1301345_I323N | HV1301345 | CH848.3.D0949.10.17chim.6R.DS.SOSIP.664_I270N |
| | HV1301345_I270D | HV1301345_I323D | HV1301345 | CH848.3.D0949.10.17chim.6R.DS.SOSIP.664_I270D |
| | HV1301345_N332A_N442A | HV1301345_N332A_N442A | HV1301345 | CH848.3.D0949.10.17chim.6R.DS.SOSIP.664_N332A_N442A |
| | HV1301345_N301A_N442A | HV1301345_N301A_N442A | HV1301345 | CH848.3.D0949.10.17chim.6R.DS.SOSIP.664_N301A_N442A |
| | HV1301345_N353K | HV1301345_N408K | HV1301345 | CH848.3.D0949.10.17chim.6R.DS.SOSIP.664_N353K |
| | HV1301345_E241K_N353K | HV1301345_E293K_N408K | HV1301345 | CH848.3.D0949.10.17chim.6R.DS.SOSIP.664_E241K_N353K |
| | HV1301345_T238K_E241T_N353K | HV1301345_T290K_E293T_N408K | HV1301345 | CH848.3.D0949.10.17chim.6R.DS.SOSIP.664_T238K_E241T_N353K |
| | HV1301345_V243T_T357K_E283T_{—} | HV1301345_V295T_T413K_E336T_{—} | HV1301345 | CH848.3.D0949.10.17chim.6R.DS.SOSIP.664_V243T_T357K_E283T_{—} |
| | T238K | T290K | | T238K |
| | HV1301345_E241T_T238K_T357K | HV1301345_E293T_T290K_T413K | HV1301345 | CH848.3.D0949.10.17chim.6R.DS.SOSIP.664_E241T_T238K_T357K |
| HV1302209 | HV1301345_N442A | HV1301345_N442A | HV1301345 | CH848.3.D0949.10.17chim.6R.DS.SOSIP.664_N442A |
| | Encounter Designs with V1 N-glycosylation Sites Mutated (note N133D, | | | |
| | N138T, N301A, N332A, and N442A are listed throughout already in HXB2 numbering) | | | |
| | HV1301345_N133D_N138T | HV1301345_N133D_N138T | HV1301345_{—} | CH848.3.D0949.10.17chim.6R.DS.SOSIP.664_N133D_N138T |
| | | | N133D_N138T | |
| | HV1301345_N133D_N138T_N353K | HV1301345_N133D_N138T_N408K | HV1301345_{—} | CH848.3.D0949.10.17chim.6R.DS.SOSIP.664_N133D_N138T_N353K |
| | | | N133D_N138T | |
| | HV1301345_N133D_N138T_E241K_{—} | HV1301345_N133D_N138T_E293K_{—} | HV1301345_{—} | CH848.3.D0949.10.17chim.6R.DS.SOSIP.664_N133D_N138T_E241K_{—} |
| | N353K | N408K | N133D_N138T | N353K |
| HV1302212 | HV1301345_N133D_N138T_T238K_{—} | HV1301345_N133D_N138T_T290K_{—} | HV1301345_{—} | CH848.3.D0949.10.17chim.6R.DS.SOSIP.664_N133D_N138T_T238K_{—} |
| | E241T_N353K | E293T_N408K | N133D_N138T | E241T_N353K |
| | HV1301345_N133D_N138T_E241T_{—} | HV1301345_N133D_N138T_E293T_{—} | HV1301345_{—} | CH848.3.D0949.10.17chim.6R.DS.SOSIP.664_N133D_N138T_E241T_{—} |
| | T238K_T357K | T290K_T413K | N133D_N138T | T238K_T357K |
| | HV1301345_N133D_N138T_V243T_{—} | HV1301345_N133D_N138T_V295T_{—} | HV1301345_{—} | CH848.3.D0949.10.17chim.6R.DS.SOSIP.664_N133D_N138T_V243T_{—} |
| | T357K_E283T_T238K | T413K_E336T_T290K | N133D_N138T | T357K_E283T_T238K |
| HV1302215 | HV1301345_N133D_N138T_N442A | HV1301345_N133D_N138T_N442A | HV1301345_{—} | CH848.3.D0949.10.17chim.6R.DS.SOSIP.664_N133D_N138T_N442A |
| | | | N133D_N138T | |
| | HV1301345_N133D_N138T_A352N_{—} | HV1301345_N133D_N138T_A407N_{—} | HV1301345_{—} | CH848.3.D0949.10.17chim.6R.DS.SOSIP.664_N133D_N138T_A352N_{—} |
| | N353K | N408K | N133D_N138T | N353K |
| | HV1301345_N133D_N138T_E241T_{—} | HV1301345_N133D_N138T_E293T_{—} | HV1301345_{—} | CH848.3.D0949.10.17chim.6R.DS.SOSIP.664_N133D_N138T_E241T_{—} |
| | N353K | N408K | N133D_N138T | N353K |
| | HV1301345_N133D_N138T_T238K_{—} | HV1301345_N133D_N138T_T290K_{—} | HV1301345_{—} | CH848.3.D0949.10.17chim.6R.DS.SOSIP.664_N133D_N138T_T238K_{—} |
| | E241T_A352N_N353K | E293T_A407N_N408K | N133D_N138T | E241T_A352N_N353K |
| | HV1301345_N133D_N138T_E241T | HV1301345_N133D_N138T_E293T | HV1301345_{—} | CH848.3.D0949.10.17chim.6R.DS.SOSIP.664_N133D_N138T_E241T |
| | | | N133D_N138T | |
| | HV1301345_N133D_N138T_E241T_{—} | HV1301345_N133D_N138T_E293T_{—} | HV1301345_{—} | CH848.3.D0949.10.17chim.6R.DS.SOSIP.664_N133D_N138T_E241T_{—} |
| | S281K | S334K | N133D_N138T | S281K |
| | HV1301345_N133D_N138T_E241T_{—} | HV1301345_N133D_N138T_E293T_{—} | HV1301345_{—} | CH848.3.D0949.10.17chim.6R.DS.SOSIP.664_N133D_N138T_E241T_{—} |
| | T355K | T411K | N133D_N138T | T355K |
| | HV1301345_N133D_N138T_E241T_{—} | HV1301345_N133D_N138T_E293T_{—} | HV1301345_{—} | CH848.3.D0949.10.17chim.6R.DS.SOSIP.664_N133D_N138T_E241T_{—} |
| | T238K_S281K | T290K_S334K | N133D_N138T | T238K_S281K |
| | HV1301345_N133D_N138T_E241T_{—} | HV1301345_N133D_N138T_E293T_{—} | HV1301345_{—} | CH848.3.D0949.10.17chim.6R.DS.SOSIP.664_N133D_N138T_E241T_{—} |
| | V243T | V295T | N133D_N138T | V243T |
| | HV1301345_N133D_N138T_T238K | HV1301345_N133D_N138T_T290K | HV1301345_{—} | CH848.3.D0949.10.17chim.6R.DS.SOSIP.664_N133D_N138T_T238K |
| | | | N133D_N138T | |
| | HV1301345_N133D_N138T_V243T | HV1301345_N133D_N138T_V295T | HV1301345_{—} | CH848.3.D0949.10.17chim.6R.DS.SOSIP.664_N133D_N138T_V243T |
| | | | N133D_N138T | |
| | HV1301345_N133D_N138T_V243T_{—} | HV1301345_N133D_N138T_V295T_{—} | HV1301345_{—} | CH848.3.D0949.10.17chim.6R.DS.SOSIP.664_N133D_N138T_V243T_{—} |
| | E241K | E293K | N133D_N138T | E241K |
| | HV1301345_N133D_N138T_V243T_{—} | HV1301345_N133D_N138T_V295T_{—} | HV1301345_{—} | CH848.3.D0949.10.17chim.6R.DS.SOSIP.664_N133D_N138T_V243T_{—} |
| | E241K_S354K | E293K_S409K | N133D_N138T | E241K_S354K |
| | HV1301345_N133D_N138T_N248C_{—} | HV1301345_N133D_N138T_N300C_{—} | HV1301345_{—} | CH848.3.D0949.10.17chim.6R.DS.SOSIP.664_N133D_N138T_N248C_{—} |
| | I273C | I327C | N133D_N138T | I273C |
| | HV1301345_N133D_N138T_N250C_{—} | HV1301345_N133D_N138T_N302C_{—} | HV1301345_{—} | CH848.3.D0949.10.17chim.6R.DS.SOSIP.664_N133D_N138T_N250C_{—} |
| | I269C | I322C | N133D_N138T | I269C |
| | HV1301345_N133D_N138T_N250C_{—} | HV1301345_N133D_N138T_N302C_{—} | HV1301345_{—} | CH848.3.D0949.10.17chim.6R.DS.SOSIP.664_N133D_N138T_N250C_{—} |
| | T266C | T320C | N133D_N138T | T266C |
| | HV1301345_N133D_N138T_L121C_{—} | HV1301345_N133D_N138T_L173C_{—} | HV1301345_{—} | CH848.3.D0949.10.17chim.6R.DS.SOSIP.664_N133D_N138T_L121C_{—} |
| | T266C | T320C | N133D_N138T | T266C |
| | HV1301345_N133D_N138T_R246C_{—} | HV1301345_N133D_N138T_R298C_{—} | HV1301345_{—} | CH848.3.D0949.10.17chim.6R.DS.SOSIP.664_N133D_N138T_R246C_{—} |
| | A276C | A329C | N133D_N138T | A276C |
| | HV1301345_N133D_N138T_R246C_{—} | HV1301345_N133D_N138T_R298C_{—} | HV1301345_{—} | CH848.3.D0949.10.17chim.6R.DS.SOSIP.664_N133D_N138T_R246C_{—} |
| | A276C_E327L | A329C_E381L | N133D_N138T | A276C_E327L |
| | HV1301345_N133D_N138T_I270N | HV1301345_N133D_N138T_I323N | HV1301345_{—} | CH848.3.D0949.10.17chim.6R.DS.SOSIP.664_N133D_N138T_I270N |
| | | | N133D_N138T | |
| | HV1301345_N133D_N138T_I270D | HV1301345_N133D_N138T_I323D | HV1301345_{—} | CH848.3.D0949.10.17chim.6R.DS.SOSIP.664_N133D_N138T_I270D |
| | | | N133D_N138T | |
| | HV1301345_N133D_N138T_N332A_{—} | HV1301345_N133D_N138T_N332A_{—} | HV1301345_{—} | CH848.3.D0949.10.17chim.6R.DS.SOSIP.664_N133D_N138T_N332A_{—} |
| | N442A | N442A | N133D_N138T | N442A |
| | HV1301345_N133D_N138T_N442A | HV1301345_N133D_N138T_N442A | HV1301345_{—} | CH848.3.D0949.10.17chim.6R.DS.SOSIP.664_N133D_N138T_N442A |
| | | | N133D_N138T | |
| | HV1301345_N133D_N138T_N301A_{—} | HV1301345_N133D_N138T_N301A_{—} | HV1301345_{—} | CH848.3.D0949.10.17chim.6R.DS.SOSIP.664_N133D_N138T_N301A_{—} |
| | N442A | N442A | N133D_N138T | N442A |
| | HV1301345_N133D_N138T_N332A_{—} | HV1301345_N133D_N138T_N332A_{—} | HV1301345_{—} | CH848.3.D0949.10.17chim.6R.DS.SOSIP.664_N133D_N138T_N332A_{—} |
| | N442A | N442A | N133D_N138T | N442A |
| | HV1301345_N133D_N138T_N301A | HV1301345_N133D_N138T_N301A | HV1301345_{—} | CH848.3.D0949.10.17chim.6R.DS.SOSIP.664_N133D_N138T_N301A |
| | | | N133D_N138T | |
| | HV1301345_N133D_N138T_N332A | HV1301345_N133D_N138T_N332A | HV1301345_{—} | CH848.3.D0949.10.17chim.6R.DS.SOSIP.664_N133D_N138T_N332A |
| | | | N133D_N138T | |
| | HV1301345_N133D_N138T_E169K_{—} | HV1301345_N133D_N138T_E169K_{—} | | |
| | N442A | N442A | | |
| | HV1301345_N133D_N138T_E169K_{—} | HV1301345_N133D_N138T_E169K_{—} | | |
| | N301A_N442A | N301A_N442A | | |
| | HV1301345_N133D_N138T_E169K_{—} | HV1301345_N133D_N138T_E169K_{—} | | |
| | N332A_N442A | N332A_N442A | | |
| | Encounter designs currently moving forward for small animal trials | | | |
| HV1302206 | HV1301345_T238K_E241T_N353K | HV1301345_T290K_E293T_N408K | HV1301345 | CH848.3.D0949.10.17chim.6R.DS.SOSIP.664_T238K_E241T_N353K |
| HV1302212 | HV1301345_N133D_N138T_T238K_{—} | HV1301345_N133D_N138T_T290K_{—} | HV1301345_{—} | CH848.3.D0949.10.17chim.6R.DS.SOSIP.664_N133D_N138T_T238K_{—} |
| | E241T_N353K | E293T_N408K | N133D_N138T | E241T_N353K |
| HV1302206_N442 | HV1301345_T238K_E241T_N353K_{—} | HV1301345_T290K_E293T_N408K_{—} | HV1301345 | CH848.3.D0949.10.17chim.6R.DS.SOSIP.664_T238K_E241T_N353K_{—} |
| | N442A | N442A | | N442A |
| HV1302212_N442 | HV1301345_N133D_N138T_T238K_{—} | HV1301345_N133D_N138T_T290K_{—} | HV1301345_{—} | CH848.3.D0949.10.17chim.6R.DS.SOSIP.664_N133D_N138T_T238K_{—} |
| | E241T_N353K_N442A | E293T_N408K_N442A | N133D_N138T | E241T_N353K_N442A |
| | Encounter Site Alanine mutations | | | |
| | HV1301345_S354A | HV1301345_S409A | HV1301345 | CH848.3.D0949.10.17chim.6R.DS.SOSIP.664_S354A |
| | HV1301345_E241A | HV1301345_E293A | HV1301345 | CH848.3.D0949.10.17chim.6R.DS.SOSIP.664_E241A |
| | HV1301345_S281A | HV1301345_S334A | HV1301345 | CH848.3.D0949.10.17chim.6R.DS.SOSIP.664_S281A |
| | HV1301345_I280A | HV1301345_I333A | HV1301345 | CH848.3.D0949.10.17chim.6R.DS.SOSIP.664_I280A |
| | HV1301345_T355A | HV1301345_T411A | HV1301345 | CH848.3.D0949.10.17chim.6R.DS.SOSIP.664_T355A |
| | HV1301345_V243A | HV1301345_V295A | HV1301345 | CH848.3.D0949.10.17chim.6R.DS.SOSIP.664_V243A |
| | HV1301345_I242A | HV1301345_I294A | HV1301345 | CH848.3.D0949.10.17chim.6R.DS.SOSIP.664_I242A |
| | HV1301345_E283A | HV1301345_E336A | HV1301345 | CH848.3.D0949.10.17chim.6R.DS.SOSIP.664_E283A |
| | HV1301345_K284A | HV1301345_K337A | HV1301345 | CH848.3.D0949.10.17chim.6R.DS.SOSIP.664_K284A |
| | HV1301345_T238A | HV1301345_T290A | HV1301345 | CH848.3.D0949.10.17chim.6R.DS.SOSIP.664_T238A |
| | HV1301345_T388A | HV1301345_T444A | HV1301345 | CH848.3.D0949.10.17chim.6R.DS.SOSIP.664_T388A |
| | HV1301345_R390A | HV1301345_R446A | HV1301345 | CH848.3.D0949.10.17chim.6R.DS.SOSIP.664_R390A |
| | HV1301345_K274A | HV1301345_K327A | HV1301345 | CH848.3.D0949.10.17chim.6R.DS.SOSIP.664_K274A |
| | HV1301345_D272A | HV1301345_D325A | HV1301345 | CH848.3.D0949.10.17chim.6R.DS.SOSIP.664_D272A |
| | HV1301345_G271A | HV1301345_G324A | HV1301345 | CH848.3.D0949.10.17chim.6R.DS.SOSIP.664_G271A |
| | HV1301345_T245A | HV1301345_T297A | HV1301345 | CH848.3.D0949.10.17chim.6R.DS.SOSIP.664_T245A |
| | HV1301345_I270A | HV1301345_I323A | HV1301345 | CH848.3.D0949.10.17chim.6R.DS.SOSIP.664_I270A |
| | HV1301345_H277A | HV1301345_H330A | HV1301345 | CH848.3.D0949.10.17chim.6R.DS.SOSIP.664_H277A |
| | HV1301345_S351A | HV1301345_S406A | HV1301345 | CH848.3.D0949.10.17chim.6R.DS.SOSIP.664_S351A |
| | HV1301345_S350A | HV1301345_S405A | HV1301345 | CH848.3.D0949.10.17chim.6R.DS.SOSIP.664_S350A |
| | HV1301345_I346A | HV1301345_I401A | HV1301345 | CH848.3.D0949.10.17chim.6R.DS.SOSIP.664_I346A |
| | HV1301345_I383A | HV1301345_I439A | HV1301345 | CH848.3.D0949.10.17chim.6R.DS.SOSIP.664_I383A |
| | HV1301345_D287A | HV1301345_D340A | HV1301345 | CH848.3.D0949.10.17chim.6R.DS.SOSIP.664_D287A |
| | HV1301345_K291A | HV1301345_K344A | HV1301345 | CH848.3.D0949.10.17chim.6R.DS.SOSIP.664_K291A |
| | HV1301345_T348A | HV1301345_T403A | HV1301345 | CH848.3.D0949.10.17chim.6R.DS.SOSIP.664_T348A |

### Naming Nomenclature for Certain Envelopes:

10.17 (Non-DT) Constructs
CH848.3.D0949.10.17chim.6R.DS.SOSIP.664 is referred to as HV1301345
CH848.3.D0949.10.17chim.6R.DS.SOSIP.664_T238K_E241T_N353K is referred to as HV1302206 and HV1301345_T238K_E241T_N353K
CH848.3.D0949.10.17chim.6R.DS.SOSIP.664_N442A is referred to as HV1302209 and HV1301345_N442A
CH848.3.D0949.10.17chim.6R.DS.SOSIP.664_T238K_E241T_N408K_N442A is referred to as HV1302206_N442A and HV1301345_T238K_E241T_N353K_N442A.
10.17 DT Constructs
CH848.3.D0949.10.17chim.6R.DS.SOSIP.664_N133D_N138T
HV1301345_N133D_N138T
CH848.3.D0949.10.17chim.6R.DS.SOSIP.664_N133D_N138T_T238K_E241T_N353K is referred to as HV1302212 and HV1301345_N133D_N138T_T238K_E241T_N353K
CH848.3.D0949.10.17chim.6R.DS.SOSIP.664_N133D_N138T_N442A is referred to as HV1302215 and HV1301345_N133D_N138T_N442A
CH848.3.D0949.10.17chim.6R.DS.SOSIP.664_N133D_N138T_T238K_E241T_N353K N442A is referred to as HV1302212_N442A and
HV1301345_N133D_N138T_T238K_E241T_N353K_N442A

TABLE 1B
Summary of envelope amino acid positions involved in antibody encounter complexes and sequence names. The first column identifies
encounter mutation amino acid position based on the first amino acid in the sequence in FIG. 21. The second column identifies encounter
amino acid position based on HXB2 numbering. Provided are non-limiting examples of changes to specific amino acid at selected position.
A skilled artisan can readily determine additional changes, based on well-known properties and considerations of nature and properties
of amino acids, at these selected positions. Non-limiting embodiments of amino acid sequences are shown in FIG. 21.

| | | | |
|---|---|---|---|
| | | PARENT | |
| CONSTRUCT | HXB2 Numbers | SEQUENCE | FULL ID |

Encounter Tryptophan Mutations

| | | | |
|---|---|---|---|
| HV1301345 | HV1301345 | HV1301345 | CH848.3.D0949.10.17chim.6R.DS.SOSIP.664 |
| HV1301345_T238K_E241T_{—} | HV1301345_T290K_E293T_{—} | HV1301345 | CH848.3.D0949.10.17chim.6R.DS.SOSIP.664 |
| N353K_D230N_E169K | N408K_D230N_E169K | | |
| HV1301345_T238K_E241T_{—} | HV1301345_T290K_E293T_{—} | HV1301345 | CH848.3.D0949.10.17chim.6R.DS.SOSIP.664 |
| N353K_D230N_H289N_P291S_{—} | N408K_D230N_H289N_P291S_{—} | | |
| E169K | E169K | | |
| HV1301345_T238K_E241T_{—} | HV1301345_T290K_E293T_{—} | HV1301345 | CH848.3.D0949.10.17chim.6R.DS.SOSIP.664 |
| N353K_N442A_D230N_E169K | N408K_N442A_D230N_E169K | | |
| HV1301345_T238K_E241T_{—} | HV1301345_T290K_E293T_{—} | HV1301345 | CH848.3.D0949.10.17chim.6R.DS.SOSIP.664 |
| N353K_N442A_D230N_H289N_{—} | N408K_N442A_D230N_H289N_{—} | | |
| P291S_E169K | P291S_E169K | | |
| HV1301345_N442A_D230N_{—} | HV1301345_N442A_D230N_{—} | HV1301345 | CH848.3.D0949.10.17chim.6R.DS.SOSIP.664 |
| E169K | E169K | | |
| HV1301345_N442A_D230N_{—} | HV1301345_N442A_D230N_{—} | HV1301345 | CH848.3.D0949.10.17chim.6R.DS.SOSIP.664 |
| H289N_P291S_E169K | H289N_P291S_E169K | | |
| HV1301345_N133D_N138T_{—} | HV1301345_N133D_N138T_{—} | HV1301345 | CH848.3.D0949.10.17chim.6R.DS.SOSIP.664 |
| N442A_D230N_E169K | N442A_D230N_E169K | | |
| HV1301345_N133D_N138T_{—} | HV1301345_N133D_N138T_{—} | HV1301345 | CH848.3.D0949.10.17chim.6R.DS.SOSIP.664 |
| N442A_D230N_H289N_P291S_{—} | N442A_D230N_H289N_P291S_{—} | | |
| E169K | E169K | | |
| HV1301345_N353K_N442A | HV1301345_N408K_N442A | HV1301345 | CH848.3.D0949.10.17chim.6R.DS.SOSIP.664 |
| HV1301345_N353K_D230N_{—} | HV1301345_N408K_D230N_{—} | HV1301345 | CH848.3.D0949.10.17chim.6R.DS.SOSIP.664 |
| E169K | E169K | | |
| HV1301345_N353K_D230N_{—} | HV1301345_N408K_D230N_{—} | HV1301345 | CH848.3.D0949.10.17chim.6R.DS.SOSIP.664 |
| H289N_P291S_E169K | H289N_P291S_E169K | | |
| HV1301345_N353K_N442A_{—} | HV1301345_N408K_N442A_{—} | HV1301345 | CH848.3.D0949.10.17chim.6R.DS.SOSIP.664 |
| D230N_E169K | D230N_E169K | | |
| HV1301345_N353K_N442A_{—} | HV1301345_N408K_N442A_{—} | HV1301345 | CH848.3.D0949.10.17chim.6R.DS.SOSIP.664 |
| D230N_H289N_P291S_E169K | D230N_H289N_P291S_E169K | | |
| HV1301345_N133D_N138T_{—} | HV1301345_N133D_N138T_{—} | HV1301345_{—} | CH848.3.D0949.10.17chim.6R.DS.SOSIP.664_{—} |
| T238K_E241T_N353K_D230N_{—} | T290K_E293T_N408K_D230N_{—} | N133D_N138T | N133D_N138T |
| E169K | E169K | | |
| HV1301345_N133D_N138T_{—} | HV1301345_N133D_N138T_{—} | HV1301345_{—} | CH848.3.D0949.10.17chim.6R.DS.SOSIP.664_{—} |
| T238K_E241T_N353K_D230N_{—} | T290K_E293T_N408K_D230N_{—} | N133D_N138T | N133D_N138T |
| H289N_P291S_E169K | H289N_P291S_E169K | | |
| HV1301345_N133D_N138T_{—} | HV1301345_N133D_N138T_{—} | HV1301345_{—} | CH848.3.D0949.10.17chim.6R.DS.SOSIP.664_{—} |
| T238K_E241T_N353K_N442A_{—} | T290K_E293T_N408K_N442A_{—} | N133D_N138T | N133D_N138T |
| D230N_E169K | D230N_E169K | | |
| HV1301345_N133D_N138T_{—} | HV1301345_N133D_N138T_{—} | HV1301345_{—} | CH848.3.D0949.10.17chim.6R.DS.SOSIP.664_{—} |
| T238K_E241T_N353K_N442A_{—} | T290K_E293T_N408K_N442A_{—} | N133D_N138T | N133D_N138T |
| D230N_H289N_P291S_E169K | D230N_H289N_P291S_E169K | | |

Tables 1A and 1B are collectively referred as Table 1.

A skilled artisan appreciates that using HXB2 numbering to identify a corresponding amino acid position in a sequence of interest is done by alignment of the sequence of interest to the standard HXB2 sequence.

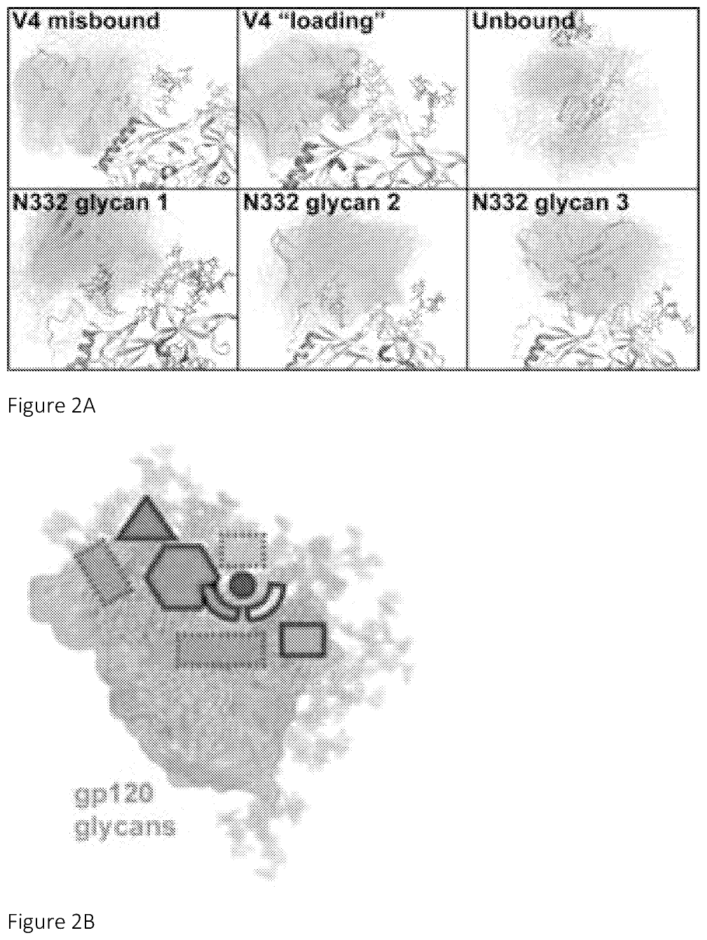

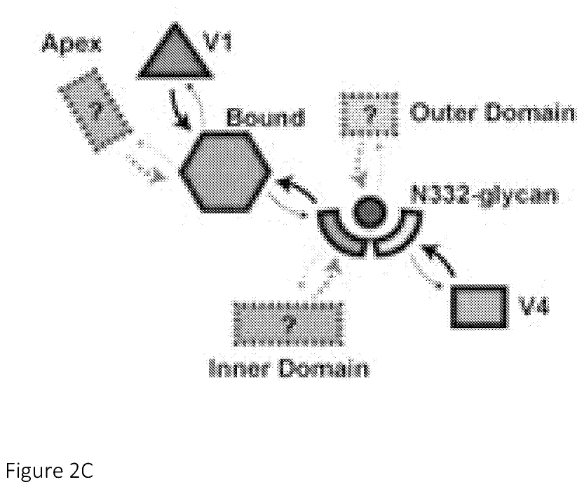

Designs under the heading “Encounter Tryptophan Mutations” were chosen based upon the N332 glycan 1-3 states in FIG. 2A (top). The V4 “loading” state accesses the bound state via these three states. These three states represent rotation about the N332 glycan beginning from a near V4 state (N332 glycan 1) rotating around to a pre-bound state (N332 glycan 3). Controlling the pathway of association changes the antibody-Env contact sites and can change vaccination outcomes. These mutations were therefore designed to interrupt these transitions by adding a bulky Tryptophan residue to sterically block this rotation pathway which can in turn block the V4 region pathway.

Designs under the “Encounter Designs with V1 N-glycosylation Sites Present” and “Encounter Designs with V1 N-glycosylation Sites Mutated” headings were chosen based upon the V4 “loading” state in FIG. 2A (top). This state has access to the bound state via the N332 glycan 1-3 states and so, making this state more available and/or stabilizing it and/or attracting it to the N332 glycan states can improve the association rate. Mutations chosen for these designs can increase attraction of the antibody to this state via electrostatic and/or polar complementarity in the Env to the antibody.

Designs under the “Encounter Site Alanine mutations” heading were selected based upon observed states in the model that were found to have access to the bound state. Alanine mutations at each contact site were prepared to selectively reduce or enhance transitions involving each site.

### Example 1

This example describes designs of HIV-1 envelopes comprising amino acid changes that improve antibody association rates by stabilizing collision induced encounter states and enhancing transition rates between encounter to bound state intermediates. In some aspects, the invention provides envelope sequence designs comprising amino acid changes at one or more positions in the envelope, wherein these envelope positions are antibody-envelope encounter sites/residues. In some embodiments, the antibody-envelope encounter residue(s) are contact residues.

### Background

N332 glycan targeting bnAbs are common yet quite diverse: Multiple bnAbs targeting the N332-glycan supersite have been identified from different infected HIV-1 individuals and simian-human (SHIV) infected macaques. These include DH270 (see Bonsignori et. al. (2017) Sci. Transl. Med 9), PGT128 (see Perchal et. al. (2011) Science 334, 1097-1103), PGT135 (see Kong et. al. (2013) Nat. Struc. Biol 20, 796-803), PGT121 (see Mouquet et. al. (2012) PNAS 109, E3268-E3277) and a new antibody recently isolated from a SHIV infected macaque, referred to as DH1030. These cover differing V_{H} and V_{L} genes and each displays a distinct angle of approach in the bound configuration. See Bonsignori et. al. (2017) Sci. Transl. Med 9; Kong et. al. (2013) Nat. Struc. Biol 20, 796-803 and Sok et. al. (2016) Immunity 45, 31-45. A large number of high-resolution crystal structures of these antibodies and of medium resolution cryo-EM structures of their bound state complexes with stabilized, soluble Envs, termed SOSIPs, have been obtained for structure-based design (see Bonsignori et. al. (2017) Sci. Transl. Med 9; Perchal et. al. (2011) Science 334, 1097-1103; Kong et. al. (2013) Nat. Struc. Biol 20, 796-803; Mouquet et. al. (2012) PNAS 109, E3268-E3277; Kong et. al. (2015) Acta Cyrstallogr. A 71, 2099-2108; Kong et. al. (2016) Nat. Comm. A 7, 12040; Stanfield et. al. (2020) Sci. Adv. 6, eaba0464; Pantophlet et. al. (2017) Nat. Comm. 8, 1601; Saunders et. al. (2019) Science 366, eaay7199; Fera et. al. (2018) Nat. Comm. 9, 1111) and can be used in this proposal as a starting point for molecular simulation. Designs aimed at eliciting bnAb responses based on these structures have shown some promise but have yet to elicit a consistent bnAb response. See Kong et. al. (2013) Nat. Struc. Biol 20, 796-803; Saunders et. al. (2019) Science 366, eaay7199; Steichen et. al. (2019) Science 366, eaax4380.

The DH270 antibody lineage is a primary target due to its relatively limited degree of somatic mutation and the detailed knowledge of Env sequence diversity in the infected patient as the lineage developed. See Bonsignori et. al. (2017) Sci. Transl. Med 9. Our recent efforts have targeted functionally critical mutations in the lineage that are improbable due to differences in activation-induced cytidine deaminase activity. See Saunders et. al. (2019) Science 366, eaay7199; Steichen et. al. (2019) Science 366, eaax4380. Indeed, HIV-1 bnAbs often contain large numbers of such mutations and are therefore of prime importance in an immunogen design context. We showed that specially designed immunogens can effectively select for such mutations in a vaccination context by altering the glycan shield. See Saunders et. al. (2019) Science 366, eaay7199. Together, the large amount of information for this class of HIV-1 targeting bnAb provides a data-rich backdrop from which to apply the new combination of methods.

Macromolecular interaction kinetics are known to play important roles in regulating diverse biological phenomena. The interaction between macromolecules is often described in terms of affinity and defined by the equilibrium dissociation constant, K_{D}. However, the description of an interaction by its affinity alone is not the complete nor necessarily the most critical aspect of an interaction. The interaction kinetics are often essential, governing the rate of association and dwell time of an interaction. Receptor-ligand interactions can have similar affinities despite displaying dramatically different association/dissociation rates. The association rate plays an important role in many processes including the mitigation of potential self-toxicity of bacterial nucleases (see Schreiber et. al. (1996) Nat. Struc. Biol. 3, 427-431; Schreiber et. al. (2009) Chem. Rev. 109, 839-860; Zhou et. al. (2013) Immunity 39, 245-258), in signaling for actin cytoskeletal reorganization (see Hemsath et. al. (2005) Mol. Cell 20, 313-324; Kim et. al. (2000) Nature 404, 151-158), and in the regulation of signal transduction by Ras GTPases (see Kiel et. al. (2008) PLOS Comp. Biol. 4, e1000245).

In each case, the same affinity can be achieved with a slower association rate and with a concomitant decrease in dissociation rate. This can, however, result in a breakdown in their respective functions. Antibody affinity maturation also involves association rate optimization. See Pecht et. al. (2018) Eur. Biophys. J. 47, 363-371; Foote et. al. (1991) Nature 352, 530-532; Xu et. al. (2015) Proteins 83, 771-780. Indeed, fast association kinetics were demonstrated to play a critical role in respiratory syncytial virus neutralization. See Bates et. al. (2013) J. Immun. 190, 3732. Macromolecular interactions form after collisions between two interaction partners. The initial contacts between interactive partners transition from these random associations through dynamic, interconverting intermediate states, termed encounter complexes, to the bound state. See Schreiber et. al. (2009) Chem. Rev. 109, 839-860.

In the case of the barnase-barstar interaction, a rapid association rate is achieved through electrostatic steering. Double-mutant cycles, which correlated residue-residue contact influences on interaction kinetics, demonstrated the importance of charged residues in both barnase and barstar in enhancing the rate of conversion from early encounter states to the transition state to binding. See Schreiber et. al. (2013) J. Mol. Biol. 248, 478-486; Harel et. al. (2013) J. Mol. Biol. 371, 180-196. Indeed, a remarkable array of barnase-barstar transient encounter states with access to higher population, late intermediate states were recently revealed by adaptive sampling molecular dynamics and Markov modelling. See Plattner et. al. (2013) Nat. Chem. 9, 1005. Spatially resolved paramagnetic relaxation enhancement based nuclear magnetic resonance experiments have provided structural encounter state details in multiple protein systems including in phosphotransferase systems (see Strickland et. al. (2019) J. Mol. Biol. 431, 2331-2342; Tang, Iwahara & Clore (2006) Nature 444, 383-386), in DNA-protein interactions (see Iwahara & Clore (2006) Nature 550, 1227-1230; Iwahara, Schweiters & Clore (2004) J. Am. Chem. Soc. 126, 12800-12808; Iwahara, Schweiters & Clore (2004) J. Am. Chem. Soc. 126, 5879-5896), in the cytochrome c-cytochrome c peroxidase interaction (see Volkov et. al. (2006) PNAS 103, 18945), and others (see Hulsker et. al. (2008) J. Am. Chem. Soc. 130, 1985-1991; Xu et. al. (2008) J. Am. Chem. Soc. 130, 6395-6403; Scanu et. al. (2013) J. Am. Chem. Soc. 130, 7681-7692); Villareal et. al. (2011) J. Am. Chem. Soc. 133, 14176-14179).

In the case of an interaction between cytochrome f and plastocyanin, PRE experiments demonstrated that, though electrostatic interactions often dominate encounter state rate enhancements, hydrophobic interactions can play an important role. See Scanu et. al. (2013) J. Am. Chem. Soc. 135, 7681-7692. Further, a recent molecular dynamics based investigation demonstrated the importance of a highly solvated transition state with few native contacts in several encounters including for barnase-barstar, the insulin dimer, Ras-Raf-RBD, RNase HI-SSB-Ct, and TYK2-Pseudokinase. See Pan et. al. (2019) PNAS 116, 4244. To be sure, methods aimed at increasing association rates do exist. See Schreiber et. al. (2009) Chem. Rev. 109, 839-860; Schreiber et. al. (2006) Proteins 235-249; Alsallaq & Zhou et. al. (2008) Proteins 71, 320-335; Acuner et. al. (2011) Proteins 24, 635-648. The methods are, however, limited to improving electrostatic steering and/or use a coarse grained approach toward identifying effective mutations. These approaches cannot provide the level of precision needed to guide selection of specific mutations. These studies demonstrate the importance of considering interaction kinetics while providing a strong theoretical background from which to develop the designs here.

### Results

High-throughput cryo-EM pipeline for rapid determination of compositionally and conformationally heterogenous structures: The advent of rapid, high-resolution data collection in the field of cryo-EM along with advances in map refinement of highly heterogenous samples has enabled studies of structural states that were not previously possible. See Scheres (2016) Methods Enzymol. Vol. 579, 125-157. We have developed a high-throughput cryo-EM structure determination pipeline, allowing us to quickly gather key structural insights into the DH270 lineage's development (FIG. 1).

These structures revealed predicted shifts in the Fab conformation (see Henderson et. al. (2019) Nat. Comm. 10, 654), validated in this investigation via ensemble map fitting and heterogenous classification. In a concurrent investigation of the CD4 interaction with a redesigned soluble Env trimer, heterogenous refinement revealed the presence of two major compositional and conformational states, one of which represents an as yet unobserved, presumably intermediate transition state. This pipeline was also used during the initial phases of the SARS-COV-2 pandemic as we worked to examine the conformation of the Spike fusion protein. See Henderson et. al. (2020) Nat. Struc. Biol.; Acharya et. al. (2020) bioRxiv 2020.2006.2030.178897; Henderson et. al. (2020) bioRxiv 2020.2006.2026.173765; Gobeil et. al. (2020) bioRxiv 2020.2010.2011.335299. Together, this robust pipeline and its effective use for interrogating multiple, highly heterogenous systems provides a strong foundation for the designs developed here.

Extensive all Atom Molecular Simulation of the Association Process for the DH270.6 bnAb and a New N332 Glycan Targeting bnAb Targeting their Cognate Antigen.

Our recently acquired structures for the DH270 lineage and a new HIV-1_N332-glycan targeting bnAb recently isolated from a SHIV infected macaque (DH1030) provided key experimental data for the construction of in-silico systems to initiate encounter complex interrogation via MD. For both DH270.6 and DH1030, simulations were performed using stable, Mano glycosylated, N and C terminal truncated Env monomer subunits (gp120s) extracted from closed state SOSIP timer structures (Note: Without wishing to be bound by theory, owing to the individual timescales of the independent simulations, the gp120 will not transition to its common, V1-V3 disordered state; this was confirmed post simulation indicating these truncated forms can act as timer surrogates such that the simulated system size is kept to a minimum to maximize the total accessible simulation time).

Simulations were performed using the CHARMM 36 FF (see Guvench et. al. (2011) J. Chem. Theory Comput. 7, 3162-3180; Best et. al. (2012) J. Chem. Theory Comput. 8, 3257-3273) in Amber (see D. A. Case et. al. (2017)) with the proteins solvated in a truncated octahedral box of TIP3P water with ions to neutralize. Hydrogen mass repartitioning (see Hopkins et. al. (2015) J. Chem. Theory Comput. 11, 1864-1874) was used to allow for a 4 fs timestep yielding simulation rates of ˜95 ns/day for each system. As the association process occurs at timescales far greater than standard MD can access on widely available compute resources, we used the adaptive sampling technique via the HTMD package. See Doerr et. al. (2016) J. Chem. Theory Comput. 12, 1845-1852. Adaptive sampling aids in the observation of rare transitions in an iterative fashion by selecting probable transition states from an ensemble of short simulations. By repeating this process, it is possible to use Markov modelling to generate long time scale equilibrium dynamics information.

We first produced 300 independently equilibrated systems initiated from a single unbound state. This provided an ensemble of potential encounter contacts and unbound antibodies for initiation of the adaptive search algorithm. Each iteration, termed an epoch, consisted of 300 independent simulations of 250 ns each and accumulated to aggregate simulation times of ˜500 us for each system, orders of magnitude beyond any previous HIV-1 Env related simulation effort. Markov models were built based upon antibody-antigen contact networks combined with a time lagged independent component projection, termed TICA (see Perez-Hernandez & Noe (2016) J. Chem. Theory Comput. 12, 6118-6129), with coarse grained models obtained using the PCCA+ method (see Deuflhard & Weber (2005) Linear Alegra Appl. 398, 161-184).

The Markov models here define the association process as a time dependent jump between the unbound, encounter, and bound states in which the probability of transition between any set of connected states, determined by the observation of transition in the simulations, is dependent only upon the current state of the model. The primary encounter states observed in the DH270.6 simulations include five distinct encounter states, two of which interact adjacent to the variable loop 4 (V4) and three sharing native contacts with the N332 glycan that are in close exchange (FIGS. 2A-C). The orientation of the antibody in one of the V4 region interactive states is oriented away from the binding site and is not found to be a part of the association path.

However, the other V4 interactive site orients the N332 glycan interactive HCRD3 loop toward the base of N332 and acts as an N332 “loading” state. The three N332 glycan associated states act together to pivot about the N332 glycan toward the bound state. Remarkably, these states were also observed in the DH1030 simulations as well as an apparent V4 region, N332 glycan “loading” state analogous to that observed in the DH270.6 path. This V4 region loading state contrasted with the DH270.6 V4 state in its location, sitting at a surface on the opposite side of the loop. In the DH270.6 simulations, a later intermediate, bound state adjacent, V_{L} interactive N442 glycan state was observed. The N442 glycan in this state occludes the N301 glycan contact site and prevents the antibody from accessing the native bound state orientation, thereby indicating it limits the association process. Together, these simulations indicate two distinct N332 glycan targeting bnAbs share similar association paths. Further, these simulations and the Markov models built from them provide key residue-residue contact information for direct experimental interrogation of the path and immunogen design.

In non-limiting embodiments the invention provides envelope designs which comprise amino acid changes at envelope residues involved in key residue-residue interactions determined by our modelling. In some embodiments, the residues are contact residues. In other embodiments, the residues are potential contact residues or nearby residues with contact or long-range interaction (i.e. electrostatic) potential. In non-limiting embodiments the invention provides envelope designs which comprise amino acid changes at envelope residues involved in key residue-residue contacts determined by our modelling. In non-limiting embodiments these envelope encounter residues positions are contact residues. In other embodiments, these envelope encounter residues can be anywhere on the envelope surface. Table 1 provides non-limiting embodiments of specific changes of these contact residues, e.g. change to Tryptophan or Alanine. The alanine mutation and tryptophan sections list sites identified as important from the simulations so far. These residues can be changed to any other suitable amino acid.

### Association Path Design of Enhanced Association Rates for DH270 Lineage Antibodies.

Our recently published vaccine design effort demonstrated the CH848 variable loop 1 (V1) glycan deleted SOSIP (referred to as the DT construct) effectively induced DH270 lineage development in a DH270 UCA knock-in mouse model. See Saunders et. al. (2019) Science 336, eaay7199. This immunogen was selective for a key improbable mutation in an early intermediate, termed 15. Several important, improbable N332-glycan contact residues in the next intermediate, 13, reside in a cleft between the heavy and light chains. This has presented a problem from a rational immunogen design perspective, as the primary tool for altering the selection of residues via vaccination is modifying residue contact properties of the antibody-immunogen complex through amino acid mutation. No clear path to directly and uniformly modify a glycan on an immunogen is currently available.

In an effort to design an immunogen selective for these residues, we examined residue contacts in the Markov model states of the DH270.6 vs. CH848 non-DT gp120 system. The model indicated the V4 “loading” state contained structural states with contacts between the N332 glycan binding cleft and the CH848 gp120. As this state is in close contact with the N332 glycan pivot intermediate, without wishing to be bound by theory, modification of the V4 encounter residues T238K_E241T_N353K can effectively accelerate the association rate for the I3 intermediate through complementary mutation in the CH848 SOSIP immunogen. Specifically, elimination of a glycan in V4 and introduction of positive charge on the immunogen can enhance association rates by increasing antibody access to the pivot state. Without wishing to be bound by theory, the V4 encounter residues that were modified led to enhanced association rates.

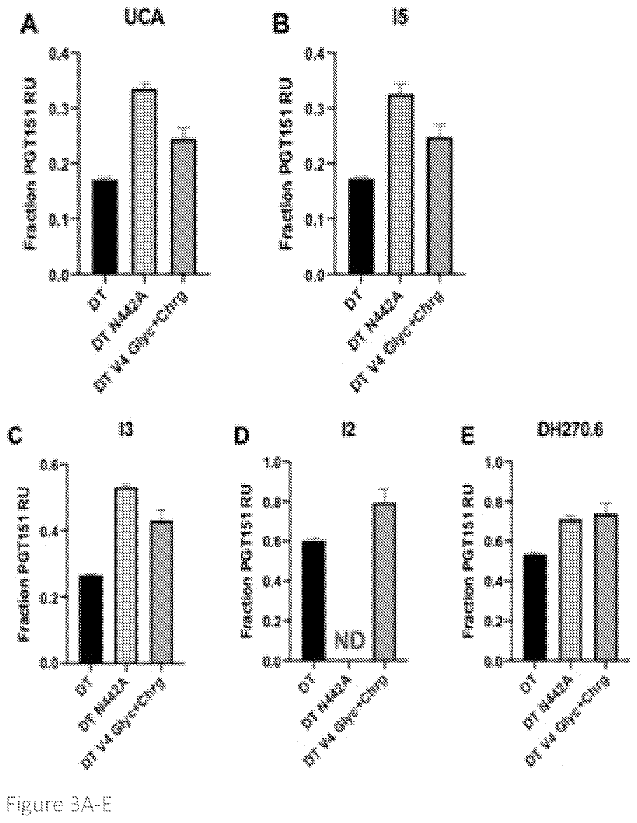

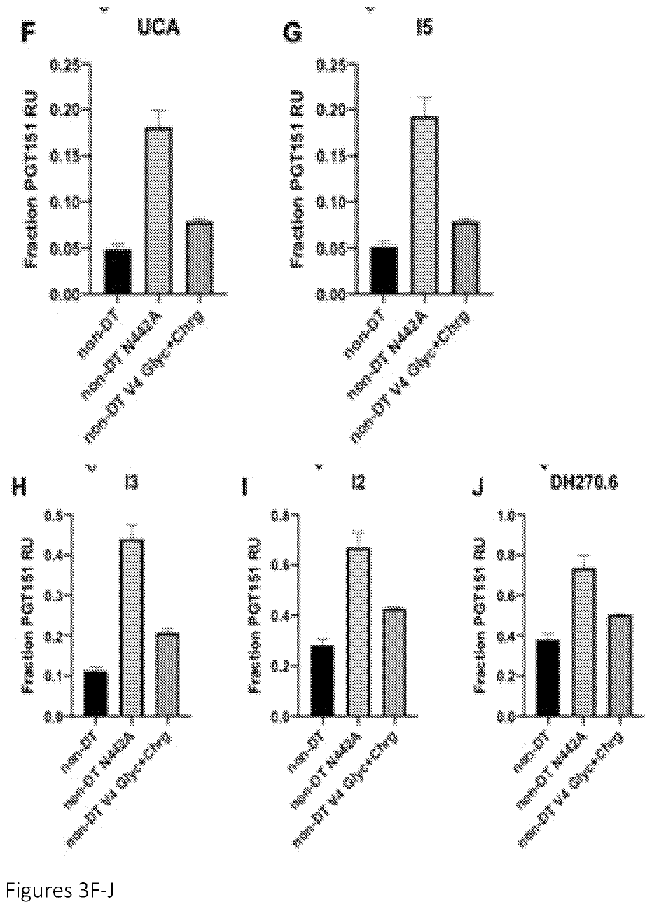

Without wishing to be bound by theory, eliminating the N442 glycan site can increase the association rate by eliminating its on-path restrictions. Towards a more complete understanding of the association path, we prepared these mutations in the context of the variable loop 1 (V1) glycan deleted and non-deleted (DT and non-DT) context. The SOSIP trimers were prepared via HEK293F transient transfection. Proteins were affinity purified using PGT151 followed by further purification using size exclusion chromatography. Trimer stability was measured using differential scanning fluorescence denaturation (Tycho NT.6, NANOTEMPER) yielding similar stabilities between parent and mutant species (denaturation inflection temperatures ˜±1-2° C.). Binding was first measured via biolayer interferometry (BLI) in triplicate to compare the constructs to their relevant unmutated form (FIGS. 3A-J).

The results are consistent with the depiction of the process in FIGS. 2A-C indicating i) the DT and non-DT N442A mutants both displayed consistently higher relative binding across the entire lineage relative to the unmutated forms, ii) this differential was greater in the non-DT construct, iii) for the V4 region mutant, the I3 intermediate shows a greater increase in binding compared to the earlier intermediates in both the DT and non-DT contexts, and iv) the differential for the V4 region mutant was greater at the I3 intermediate for the non-DT form. We next examined the interaction kinetics for the immunogen via surface plasmon resonance (SPR). The results were consistent with the binding results in direction and magnitude of the effect and indicated that the effect is indeed specific to the association rate. These results demonstrate i) the molecular simulation approach used here effectively reports on encounter states, ii) path redundancy appears to develop at intermediate I3, and iii) mutagenesis is an effective path toward modifying the encounter ensemble.

Structural ensembles from each association state in the Markov model were visualized to examine contacts in each state. Based upon visual inspection, specific residues in the antibody or Env were examined in the Markov model for their relative probabilities within the relevant state. Residues near functional improbable mutations (See PCT/US2017/054956) that are critical for neutralization breadth were monitored as well. Specifically, the light chain L48Y and heavy chain R98T residues were monitored as these occur in the I3 intermediate. The V4 “loading” state was identified as a prime target as it was 1) connected to paths reaching the bound state and 2) displayed states that were near the L48 and R98 mutation sites. Sites of contact or potential contact based upon proximity were mutated on the Env to complement observed or potential contact residues on the antibody by charge and/or polarity modification. An electrostatic surface at the L48y/R98T region of the antibody indicated this site is negatively charged and so mutation to positively charged Lysine was used at several sites to enable longer range attraction.

In some embodiments, a potential site of contact is defined as a residue on the Env that is within roughly 15 Å of the encounter ensemble that can, in principle due to the variability of the encounter structures, be induced to form a contact.

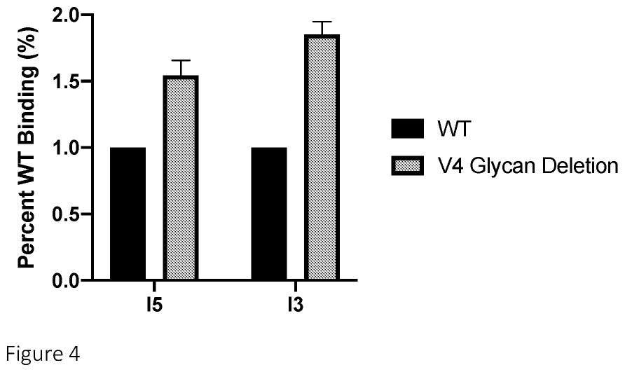

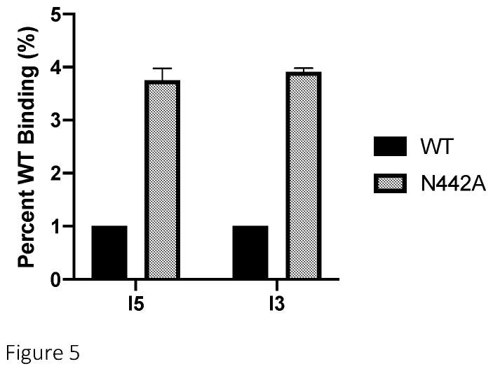

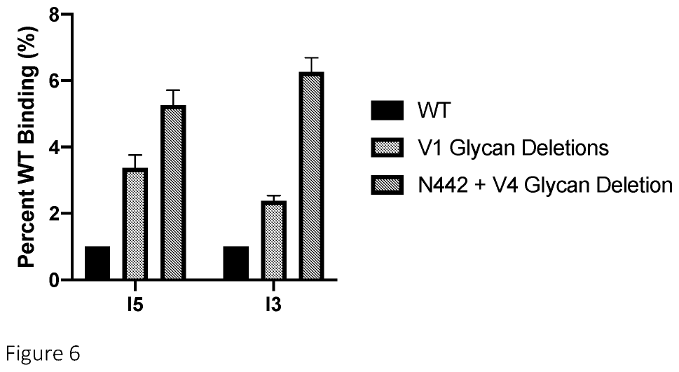

FIG. 1-6 show strategies to design germline targeting and sequential immunogens for a BnAb inducing HIV Vaccine. Our approach uses molecular dynamics simulation to investigate interactions between antibody and antigen. This is a theoretical method combining Newtonian mechanics with a numerical description of atomic interactions to calculate the motion of molecules at the atomic scale. We conducted simulations of DH270.6 association process. FIGS. 1, 4-6 provide insight in how the antibody manages the glycan shield of the HIV-1 envelope. The DH270 antibody locates the envelope surface, and needs to find a way to move through the glycan shield to the binding site after initial contact. Here, it gets through the shield but enters a maze-like environment with moving glycan walls; these include glycans at positions N156, N301, N332, and N442. We asked which contact sites can reach the bound state to help identify sites that can be modified to enhance antibody binding. FIG. 4 shows that distant encounter contact sites enable association. A common encounter site near the fourth variable loop, V4, has access to the bound state. Deletion of a V4 glycan at position N408 makes this state easier to collide with and so was predicted to enhance association rates through two important improbable DH270 lineage mutations involved in this encounter, heavy chain R98T and light chain L48Y. The envelope with this glycan deletion showed improved binding to I5 and the R98T+L48Y containing I3 intermediate as predicted. FIGS. 5 and 6 show that there are additional barriers, and removing these additional barriers improves association. FIG. 5 shows that removal of glycan N442 improved binding to I5 and I3. FIG. 6 shows that combined removal of N442 and V4 glycan deletion improved binding to I5 and I3.

For CH0848 10.17DT SOSIP sequence see WO2018/161049, incorporated by reference in its entirety.

For non-limiting examples of hole-filled CH848 703010848.3.d0949.10.17 envelopes see WO/2017152146 and WO2018/161049, inter alia without limitation, FIGS. 44A-D, [0091], incorporated by reference in its entirety.

The immunogens of the invention can be delivered by any suitable mechanism.

In non-limiting embodiments, these can be Adeno-associated virus (AAV) vectors, non-replicating viral vectors, any of which can provides sustained expression of the immunogen.

In some embodiments, the vector can transduce dendritic cells, which present the transgene (immunogen) in complex with MHCII to naïve T cells.

Constant antigen production can lead to improved clonal persistence, enhanced germinal center reactions, and higher somatic mutation.

In some embodiments, a multivalent mixture can be used to mimic chronic HIV-1 infection.

In certain embodiments, the immunogens can be multimerized.

Any of the inventive envelope designs can be tested functionally in any suitable assay. Non-limiting assays including analysis of antigenicity or immunogenicity.

Affinity and kinetics studies are summarized in FIGS. 22-24. These data show that the encounter designs improve association rates for I5, I3, I2 and DH270.6 antibodies.

### Example 2 Animal Study

The immunogens of the invention can be used to immunize any suitable animal model, including mice, non-human primates.

In non-limiting examples, the mouse model is a transgenic mouse which comprises an Ig gene(s) which encode antibody DH270-UCA, antibody DH270-13, antibody DH270-15, antibody DH270.6, or any other suitable antibody intermediate.

Various animal studies are summarized starting with FIG. 25. These studies include immunization encounter designs of mice which have various DH270 lineage antibodies, e.g. IA4 knock-in or UCA3 knock-in. Analyses of these studies included serum binding, serum neutralization, serum blocking, antibody isolation and analyses, and NGS analyses if improbable mutations. These studies demonstrate that envelope encounter designs, including without limitation HV1302206_N442A perform better as boost immunogens compared to envelopes which lack the encounter designs. For example, FIG. 34 demonstrates that HV1302206_N442A (study Mu567 group 1) immunized set are as effective as or better at inducing neutralization critical, improbable mutations G57R, R98T and the combined G57R/R98T mutations in the heavy chain and the L48Y mutation in the light chain, consistent with the goal to design immunogens which increase frequency of R98T and L48Y mutation.

Additional animal studies will include comparison of the HV1302206_N442A design boost to the 10.17-DT Nano particle prime with its parent, unmutated HV1301345 as a boost to the 10.17-DT nanoparticle prime in the DH270 UCA3 knock in mice. We will determine whether the HV1302206_N442A boost more effectively selects for the improbable R98T and L48Y mutations compared to HV1301345 boost. Additional studies will examine whether the HV1302206_N442A design is more effective as a boost to a 10.17-DT nanoparticle prime that is first boosted with the HV1301345 construct as well as whether boosting from the 10.17-DT nanoparticle first with the HV1302206_N442A and following with HV1302206 more effectively selects for the R98T and L48Y mutations. Finally, we will perform each of these studies using mRNA versions of these constructs to determine whether mRNA vaccination is more effective in selecting the R98T and L48Y mutations compared to protein immunization.

What is claimed is:

1. A recombinant HIV-1 envelope selected from the envelopes listed in FIG. 7, FIG. 21 or Table 1.

2. A composition comprising the envelope of claim 1 and a carrier, wherein the envelope is a protomer comprised in a trimer.

3. The composition of claim 1, wherein the envelope is CH848.3.D0949.10.17chim.6R.DS.SOSIP.664_T238K_E241T_N408K_N442A.

4. A composition comprising a nanoparticle and a carrier, wherein the nanoparticle comprises any one of the envelopes of claim 1.

5. The composition of claim 4, wherein the nanoparticle is a ferritin self-assembling nanoparticle.

6. A composition comprising a nanoparticle and a carrier, wherein the nanoparticle comprises any one of the trimers of claim 2.

7. The composition of claim 6, wherein the nanoparticle is a ferritin self-assembling nanoparticle.

8. The composition of claim 7, wherein the nanoparticle comprises multimers of trimers.

9. The composition of claim 7, wherein the nanoparticle comprises 1 to 8 trimers.

10. A method of inducing an immune response in a subject comprising administering in an amount sufficient to affect such induction an immunogenic composition comprising any one of the recombinant envelopes of claim 1.

11. The method of claim 10, wherein the composition is administered as a prime.

12. The method of claim 10, wherein the composition is administered as a boost.

13. A nucleic acid encoding any of the recombinant envelopes of claim 1.

14. A composition comprising the nucleic acid of claim 13 and a carrier.

15. The composition of claim 14, wherein the nucleic acid is modified mRNA formulated in LNPs.

16. A method of inducing an immune response in a subject comprising administering in an amount sufficient to affect such induction an immunogenic composition comprising the nucleic acid of claim 13.

17. The method of claim 16, wherein the composition is administered as a boost.
